(19) United States
(12) Patent Application Publication
(10) Pub. No.: US 2018/0215740 A1
(43) Pub. Date: Aug. 2, 2018

(54) PARA-SUBSTITUTED INDANYL AND TETRALINYL DERIVATIVES

(71) Applicant: BASF SE, Ludwigshafen am Rhein (DE)

(72) Inventors: Gopal Krishna Datta, Goettingen (DE); Pascal Bindschaedler, Roemerberg (DE); Wolfgang von Deyn, Neustadt (DE); Michael Rack, Eppelheim (DE); Karsten Koerber, Eppelheim (DE)

(21) Appl. No.: 15/746,029

(22) PCT Filed: Jul. 13, 2016
(86) PCT No.: PCT/EP2016/066603

§ 371 Date: Jan. 19, 2018

(30) Foreign Application Priority Data
Jul. 22, 2015 (EP) 15177883.4

(51) Int. Cl. C07D 409/12; A01N 43/54; A01N 43/40; C07D 333/24; A01N 43/10; A01N 43/20; C07D 413/12; A01N 43/80; C07D 261/04; C07D 403/12; C07D 207/20; A01N 43/36; C07D 417/12; A01N 43/78

(52) U.S. Cl. CPC C07D 409/12; A01N 43/54; A01N 43/40; C07D 333/24; A01N 43/10; A01N 43/20; A01N 43/78; A01N 43/80; C07D 261/04; C07D 403/12; C07D 207/20; A01N 43/36; C07D 417/12; C07D 413/12

(57) ABSTRACT

Compounds of formula I and formula II

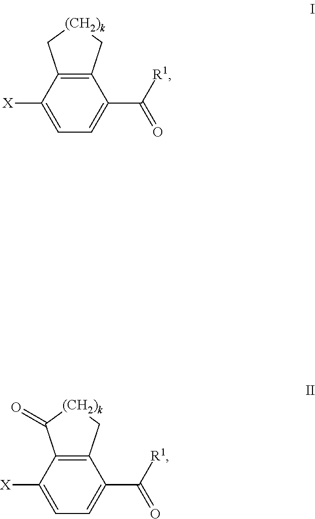

their intermediates, as well as processes for the preparation of compounds of formula I and formula II, are disclosed. Use of compounds of formula I for the production of compounds of formula VI

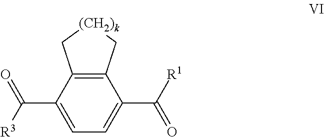

is also disclosed. Additionally, the use of compounds of formula I or formula II for the production of active compounds is disclosed.

The present invention relates to intermediate compounds of formula I

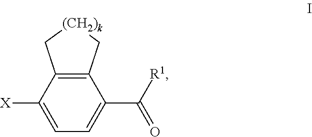

wherein the variables have the following meaning:
X halogen;
R¹ H, OR¹¹, or NR¹²R¹³;
R¹¹ a) H;
   b) C₁-C₆-alkyl, C₃-C₈-cycloalkyl, C₂-C₆-alkenyl, C₃-C₈-cycloalkenyl, C₂-C₆-alkynyl; which groups are unsubstituted, or substituted by halogen, CN, NO₂, S(O)ₘR^{A}, OR^{B}, NR^{B}R^{C}, S(O)ₘNR^{B}R^{C}, Si(R^{B})₂R^{C}, C(═O)R^{B}, C(═O)NR^{B}R^{C}, C(═O)OR^{B}, C(═S)R^{B}, C(═S)NR^{B}R^{C}, C(═S)OR^{B}, C(═S)SR^{B}, C(═NR^{B})R^{C}, C(═NR^{B})NR^{C}R^{D};
   c) phenyl, which is unsubstituted, or substituted by R^{A}; or
   d) a 3-, 4-, 5-, 6-, or 7-membered saturated, partially unsaturated, or fully unsaturated heterocycle, which heterocycle comprises one or more, same, or different heteroatoms O, N(O)ₙ, or S(O)ₘ;
   wherein
   R^{A} a) C₁-C₆-alkyl, C₃-C₈-cycloalkyl-C₁-C₄-alkyl, C₁-C₄-alkyl-C₃-C₈-cycloalkyl, C₃-C₈-cycloalkyl, C₂-C₆-alkenyl, C₂-C₆-alkynyl, phenyl;
      which groups are unsubstituted, or substituted by halogen, CN, OH, NO₂, phenyl, or C₁-C₆-alkyl-phenyl; or
      b) a 3-, 4-, 5-, 6-, or 7-membered saturated, partially unsaturated, or fully unsaturated heterocycle, which heterocycle comprises one or more, same, or different heteroatoms O, N(O)ₙ, and S(O)ₘ, wherein none, one, or more ring members are replaced by C(═O), or C(═S), and which heterocycle is unsubstituted, or substituted with halogen, CN, N₃, NO₂, SCN, SF₅, C₁-C₆-alkyl, C₁-C₆-alkoxy, C₁-C₆-haloalkyl, C₁-C₆-alkoxy-C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, or C₂-C₆-haloalkynyl;
   R^{B}, R^{C}, R^{D} are independently from one another, as defined for R^{A}, or H; or two substituents R^{B}, R^{C}, or R^{D}, together with the atom, or the atoms to which they are bound, form a 3, 4, 5, 6, or 7-membered saturated, partially unsaturated, or fully unsaturated carbocycle, or heterocycle, which cycles are unsubstituted, or substituted by R^{A}, and wherein the heterocycle comprises one, or more, same, or different heteroatoms O, N(O)ₙ, or S(O)ₘ, and wherein none, one, or more ring members are replaced by C(═O), or C(═S);
R¹² H, C₁-C₆-alkyl, C₂-C₆-alkenyl, C₂-C₆-alkynyl, C₃-C₈-cycloalkyl, phenyl; which groups are unsubstituted, or substituted by R^{E};
R¹³ a) H, C₁-C₆-alkyl, C₁-C₆-alkoxy, C₂-C₆-alkenyl, C₂-C₆-alkynyl, C₃-C₈-cycloalkyl, C₃-C₈-cycloalkyl-C₁-C₆-alkyl, phenyl; which groups are unsubstituted, or substituted by R^{E};
   b) a group Z-A, wherein Z is a chemical bond, CH₂, CH₂CH₂, or C═O; and A is a 3-, 4-, 5-, 6- or 7-membered saturated, partially unsaturated, or fully unsaturated heterocycle, which heterocycle is unsubstituted, or substituted by R^{F} and comprises one, or more, same, or different heteroatoms O, N(O)ₙ, and S(O)ₘ, and wherein none, one, or more ring members are replaced by C(═O), or C(═S), C(═NR^{B}), or C(═NOR^{B});
   c) a group S(O)ₘR^{A}, S(O)ₘN(R^{B})R^{C}, N(R^{B})R^{C}, N(R^{B})C(═O)OR^{C}, N(R^{B})C(═O)N(R^{C})R^{D}, N(R^{B})C(═S)OR^{C}, N(R^{B})C(═S)N(R^{C})R^{D}, C(═O)N(R^{B})R^{C}, C(═S)N(R^{B})R^{C}, C(═O)OR^{A}, C═NOR^{A}, C═NR^{A}R^{B}, C═NR^{B}R^{C}; or
   wherein R¹² and R¹³, together with the N-atom to which they are bound, form a 3, 4, 5, 6, or 7-membered saturated, partially unsaturated, or fully unsaturated carbocycle, or heterocycle, which cycles are unsubstituted, or substituted by R^{F} and comprises one, or more, same, or different heteroatoms O, N(O)ₙ, and S(O)ₘ, and wherein none, one, or more ring members are replaced by C(═O), or C(═S), C(═NR^{B}), or C(═NOR^{B});
or wherein R¹² and R¹³, together with the N-atom to which they are bound, form a group ═S(R^{B})R^{C}, ═NR^{B}, ═NOR^{B}, or ═NN(R^{B})R^{C};
R^{E} a) halogen, CN, N₃, NO₂, SCN, SF₅, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, Si(R^{B})₂R^{C}, OR¹¹, OSO₂R^{A}, S(O)ₘR^{A}, S(O)ₘN(R^{B})R^{C}, N(R^{B})R^{C}, C(═O)N(R^{B})R^{C}, C(═O)N(R^{B})N(R^{C})R^{D}, C(═O)NOR^{B}, C(═S)N(R^{B})R^{C}, C(═O)OR^{A};
   b) phenyl, which is unsubstituted, or substituted by R^{A}; or
   c) two substituents R^{E}, together with the atom, or the atoms to which they are bound, form a 3, 4, 5, 6, or 7-membered saturated, partially unsaturated, or fully unsaturated carbocycle, or heterocycle, which cycles are unsubstituted, or substituted by R^{A}, and wherein the heterocycle comprises one, or more, same, or different heteroatoms O, N(O)ₙ, and S(O)ₘ, and wherein none, one, or more ring members are replaced by C(═O), or C(═S), C(═NR^{B}), or C(═NOR^{B});
R^{F} a) halogen, CN, N₃, NO₂, SCN, SF₅, C₁-C₆-alkyl, C₁-C₆-haloalkyl, C₁-C₆-alkoxy-C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, Si(R^{B})₂R^{C}, OR¹¹, OSO₂R^{A}, S(O)ₘR^{A}, S(O)ₘN(R^{B})R^{C}, N(R^{B})R^{C}, C(═O)N(R^{B})R^{C}, C(═O)N(R^{B})N(R^{C})R^{D}, C(═O)NOR^{B}, N(R^{B})R^{C},
   C(═S)N(R^{B})R^{C}, C(═O)OR^{A};
   b) phenyl, which is unsubstituted, or substituted by R^{A}; or
   c) two substituents R^{F}, together with the atom, or the atoms to which they are bound, form a 3, 4, 5, 6, or 7-membered saturated, partially unsaturated, or fully unsaturated carbocycle, or heterocycle, which cycles are unsubstituted, or substituted by R^{A}, and wherein the heterocycle comprises one, or more, same, or different heteroatoms O, N(O)ₙ, and S(O)ₘ, and wherein none, one, or more ring members are replaced by C(═O), or C(═S), C(═NR^{B}), or C(═NOR^{B});
k 1, or 2
m 0, 1, or 2; and
n 0, or 1.

The invention also relates to a process for the production of compounds I by reaction of compounds II with a reducing agent

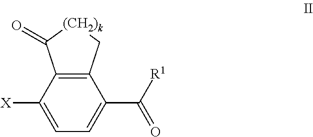

wherein all variables have a meaning as defined for compounds I.

The invention also relates to the production of compounds II by reaction of compounds V

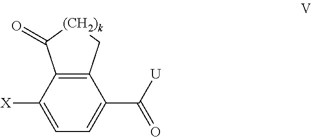

with H₂O, R¹¹OH, or NHR¹²R¹³; wherein compounds V are produced by reaction of compounds III

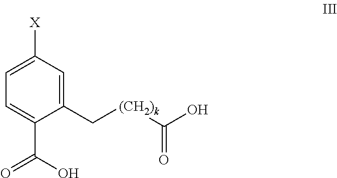

with a halogenating agent, followed by cyclization in the presence of a Lewis acid; wherein all substituents in compounds of formulae II, III, and V are defined as for compounds of formula I, and wherein U is halogen.

The invention also relates to the production of compounds III by reaction of compounds IVa, IVb, or IVc with hydrogen, followed by hydrolysis.

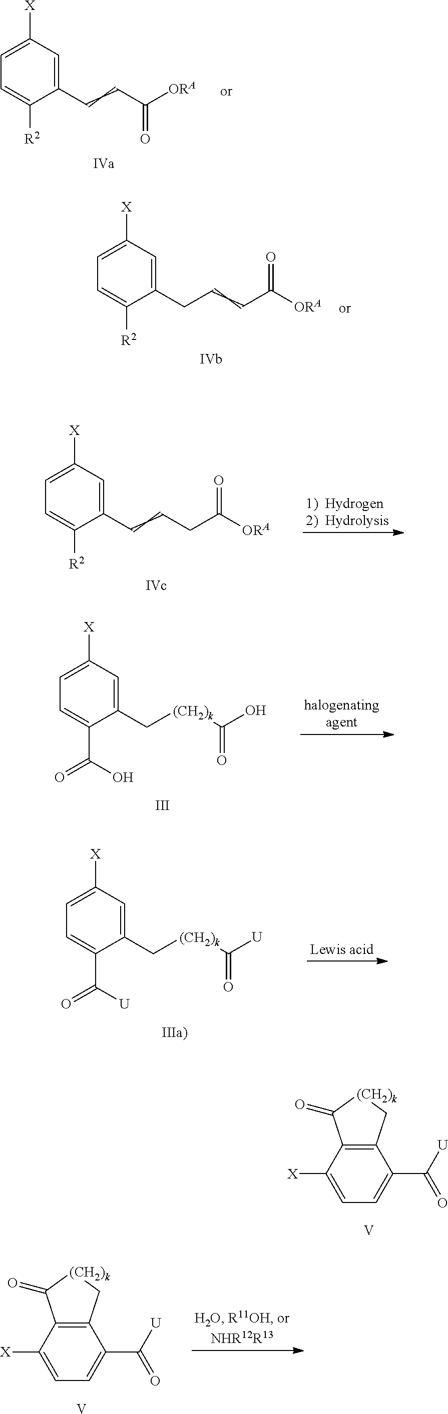

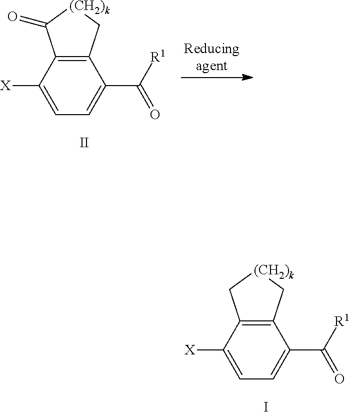

R² in compounds IVa, IVb, and IVc is CN, or C(═O)OR^{A}; U in compounds IIIa), and V is halogen; and all other substituents in compounds I, II, III, IIIa, IVa, IVb, IVc, and V have a meaning as defined for compounds I. The invention further relates to compounds III, wherein X is halogen, preferably Cl, Br, or I, in particular Br, or I, and especially Br.

The invention further relates to intermediate compounds V, which are intermediates in the production of compounds II from compounds III.

The invention also relates to the use of compounds I for the production of compounds VI,

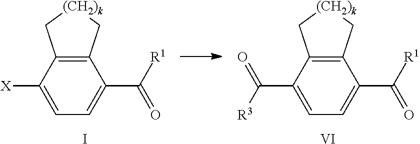

wherein R¹ and k have a meaning as defined for compounds I, and R³ is H or CH₃.

All other variables in formulae II to VI have a meaning as defined for compounds I. If not otherwise stated, this shall be the case of all depicted structures—in case the variables are present—throughout this text. Embodiments and preferences of variables defined for specific compounds are also embodiments and preferences of the variables of same nomenclature in all other compounds.

Throughout the text, the expressions “compounds”, and “compounds of formula” are equivalent expressions with the same meaning.

The invention also relates to production processes, wherein the reaction steps for the preparation of compounds I, of compounds II, or of compounds VI are carried out in a one-pot synthesis.

Indanyl and tetralinyl derivatives of formulae I and V are novel. They are valuable intermediates for the manufacture of active ingredients and fine chemicals.

Compounds V enable further conversion to a large variety of 4,7-disubstituted indane and 5,8-disubstituted tetralinyl derivatives by reaction of the activated acid moiety C(═O)U in a one-step process. Exemplary for the range of accessible compound classes are carboxylic acids, esters, amides, aldehydes, ketones, and halogens, at the carbon 4 of the indane, or the carbon 5 position of the tetralinyl derivatives.

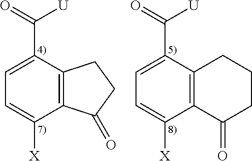

Compounds I representing the aldehyde, carboxylic acid, ester, and amide conversion products of compounds V, are equally versatile intermediates for the manufacture of active ingredients and fine chemicals. Their asymmetric substitution pattern allows for a directed manipulation to key building blocks in industrial scale manufacture of active ingredients, such as compounds VI.

Compounds VI are known from prior art WO2015/128358, where the production of compounds VI involves precursor molecules, which are derivatives of trifluoromethane sulfonic acid (hereinafter named triflates):

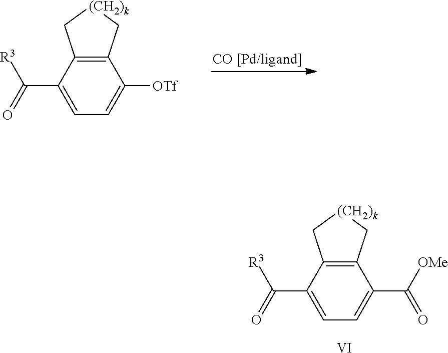

Triflates are expensive to produce and corrosive towards production plants. Triflates also hold a poor atom economy in chemical reactions, which translates to an increased amount of waste and a low environmental sustainability. The production process involving triflates is also not versatile in terms of a broad product spectrum, as only esters and carboxylates can be produced, whereas amides afford an additional coupling step.

It was therefore the objective of the invention to supply reaction intermediates, processes for their production, as well as processes for their conversion to compounds VI, which are suitable for industrial-scale production and where the abovementioned disadvantages of prior art are avoided. It was also an object of the invention to supply a versatile method and intermediates for making accessible a broad spectrum of downstream active ingredients and fine chemicals.

The objective was solved by compounds I, and V, as described above, which may be produced and further converted to compounds VI without application of triflates. Compounds I, V, and VI are key intermediates for a broad range of indanyl and tetralinyl derivatives that are crucial for active ingredient manufacture.

The reactions are economically advantageous i.a. by being characterized in high yield, high selectivity, little side products, cost effectiveness, industrial scale applicability, and little amount of waste material.

Compounds I are produced by reaction of compounds of formula II with a reducing agent.

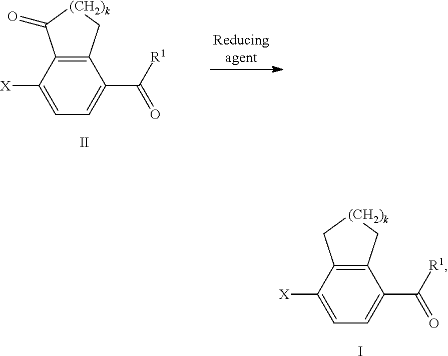

wherein all variables have a meaning as defined for compounds I.

Typical reducing agents are metals, metal salts, inorganic hydrides, and alcohols, preferably metals and inorganic hydrides, more preferably transition metals and inorganic hydrides. In another embodiment, reducing agents are metals, inorganic hydrides, and alcohols, preferably metals and inorganic hydrides, more preferably transition metals and inorganic hydrides.

In one embodiment, compounds I can be directly produced from compounds II. In another embodiment, compounds I are first reacted with a reducing agent, and then dehydroxylated, as described below.

In one embodiment, compounds I are produced by reduction of compounds II with a metal, or a metal salt, at a pH below 7.0, preferably with Zn, or a Sn(II)-salt. In another embodiment, compounds I are produced by reduction of compounds II with a metal, at a pH below 7.0, preferably with Zn.

This process is usually carried out at temperatures of from 0 to 60° C., preferably from 15 to 35° C., in a protic solvent, in the presence of an acid.

Suitable protic solvents are H₂O, or aliphatic C₁-C₄-alcohols, such as CH₃OH, CH₃CH₂OH, CH₃CH₂CH₂OH, CH₃CH(OH)CH₃, CH₃(CH₂)₃OH, and C(CH₃)₃OH, preferably H₂O, or CH₃OH. It is also possible to use mixtures of the solvents mentioned.

Suitable acids are in general inorganic acids such as hydrofluoric acid, hydrochloric acid, hydrobromic acid, sulphuric acid und perchloric acid, as well as organic acids such as formic acid, acetic acid, propionic acid, oxalic acid, toluene sulphonic acid, benzene sulphonic acid, camphor sulphonic acid, citric acid, and trifluoro acetic acid.

In a first step, the metal, which may be applied as powder, is usually amalgamated with a mercury salt under acidic conditions in H₂O. Usually, the molar ratio of the metal to compounds II is from 1:1 to 50:1, preferably from 5:1 to 20:1, and most preferably from 10:1 to 20:1. The reaction system may then be acidified with an acid to a pH below 5.0, preferably below 3.0, more preferably below 1.0. The pH may range from −3 to 5, preferably from −2 to 3, and especially preferably from −2 to 0.

Subsequently, compounds II may be added and reacted under reflux to yield compounds I.

In another embodiment, compounds II are reduced by reaction with an inorganic hydride to compounds IIa:

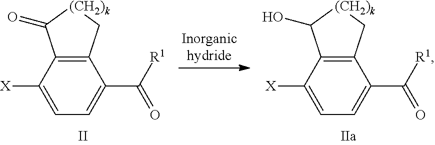

wherein all variables have a meaning as defined for compounds II.

This process is usually carried out at temperatures of from −10 to 50° C., preferably from −5 to 40° C., in a protic, or apolar solvent. In one embodiment, the reaction temperature is kept from −10 to 10° C., and then raised to 20 to 25° C., where it is kept for at least 60 minutes before reaction work-up

Suitable protic, or apolar solvents are aliphatic hydrocarbons, preferably C₅-C₁₆-alkanes, such as pentane, hexane, cyclohexane, and petrol ether; aromatic hydrocarbons, preferably C₆-C₁₀-aromatic hydrocarbon, such as toluene, o-, m-, and p-xylene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, tert-butylmethylether (MTBE), ethylene glycol dimethylether (DME), dioxane, anisole, and tetrahydrofurane (THF); alcohols, preferably C₁-C₄-alcohols, such as CH₃OH, CH₃CH₂OH, CH₃CH₂CH₂OH, CH₃CH(OH)CH₃, CH₃(CH₂)₃OH, and C(CH₃)₃OH; or H₂O.

Preferred solvents are protic solvents, more preferably H₂O, or C₁-C₄-alcohols, such as H₂O, CH₃OH, CH₃CH₂OH, CH₃CH(OH)CH₃, most preferably H₂O, CH₃OH, or CH₃CH₂OH, especially preferably H₂O, or CH₃OH, and in particular CH₃OH. It is also possible to use mixtures of the solvents mentioned.

Suitable inorganic hydrides are NaBH₄, LiAlH₄, diisobutylaluminium hydride (DIBAL-H), or its homogeneous salts, preferably NaBH₄ and DIBAL-H, more preferably NaBH₄.

Preferably, the reaction with an inorganic hydride may involve the addition of a Lewis acid.

Lewis acids are protic acids, such as trifluoroacetic acid, CH₃SO₃H, or polyphosphoric acid; aprotic inorganic salts of metals of groups 13 or 14, and transition metals of period 4, such as FeCl₃, FeBr₃, AlF₃, AlCl₃, AlBr₃, SbF₅, SbCl₅, BiF₃, BiCl₃, TiCl₄, ZnCl₂, SnCl₄, BF₃, BCl₃, BBr₃, ZrCl₄; or aprotic and metalorganic compounds metals of groups 13 or 14, and transition metals of period 4, such as Al(CH₃)₃, Al(CH₂CH₃)₃, B(CH₃)₃; moreover polyphosphate ester, trimethylsilyl polyphosphate. Preferred Lewis acids are trifluoroacetic acid and AlCl₃, more preferably trifluoroacetic acid.

Preferably, the reaction with an inorganic hydride may involve the previous conversion of compounds II to a hydrazone by reaction with hydrazine, or a derivative. Suitable hydrazine derivatives are p-toluene sulfone hydrazine, or methyl sulfone hydrazine, preferably p-toluene sulfone hydrazine.

Compounds II and the inorganic hydride are generally reacted with one another in equimolar amounts. It may be advantageous to employ an excess of the inorganic hydride, e.g. with a ratio from 1:1 to 10:1, preferably from 1:1 to 5:1, more preferably from 1:1 to 2:1.

In another embodiment, compounds II are reduced by reaction with an alcohol to compounds IIa.

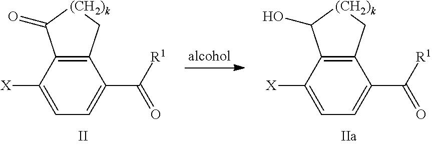

Production processes of this type are generally known as Meerwein-Ponndorf-Verley-Reductions, whose general reaction conditions are described i.a. in Jin et al., Org. Process Res. Dev., 2006, 10 (5), pp 1032-1053.

This production is usually carried out by reaction of compounds II with an alcohol, optionally in an inert solvent, in the presence of a metal alcoholate.

Suitable alcohols are C₁-C₄-alcohols, such as CH₃OH, CH₃CH₂OH, CH₃CH₂CH₂OH, CH₃CH(OH)CH₃, CH₃(CH₂)₃OH, or C(CH₃)₃OH, preferably CH₃CH₂CH₂OH or CH₃CH(OH)CH₃, more preferably CH₃CH(OH)CH₃.

Suitable inert solvents are aliphatic hydrocarbons, preferably C₅-C₁₆-alkanes, such as pentane, hexane, cyclohexane, and petrol ether; aromatic hydrocarbons, preferably C₆-C₁₀-aromatic hydrocarbons, such as toluene, o-, m-, and p-xylene; halogenated hydrocarbons, preferably halogenated C₁-C₆-alkanes or halogenated C₆-C₁₀-aromatic hydrocarbons, such as CH₂Cl₂, CHCl₃, and chlorobenzene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, dioxane, anisole, and THF; nitriles, preferably C₁-C₆-nitriles such as CH₃CN, and propionitrile. Preferred solvents are aliphatic hydrocarbons, preferably C₅-C₁₆-alkanes, such as pentane, hexane, cyclohexane, and petrol ether, or aromatic hydrocarbons, preferably C₆-C₁₀-aromatic hydrocarbons, such as toluene, o-, m-, and p-xylene; halogenated hydrocarbons, preferably halogenated C₁-C₆-alkanes and halogenated C₆-C₁₀-aromatic hydrocarbons, such as CH₂Cl₂, CHCl₃, and chlorobenzene. It is also possible to use mixtures of the solvents mentioned. In one embodiment, the production is carried out with an alcohol and without a solvent.

Suitable metal alcoholates are usually the C₁-C₆-alcoholates of transition metals of the 4^{th} period, such as Zn(OCH₃)₂, Zn(OCH₃CH₂)₂, Zn[OCH(CH₃)₂]₂, Fe(OCH₃)₃, Fe(OCH₂CH₃)₃, Fe(OCH(CH₃)₂)₃ or metals of group 13, such as Al(OCH₃)₃, Al(OCH₂CH₃)₃, Al(OCH(CH₃)₂)₃. Preferably, metal alcoholates are C₁-C₆-alcoholates, preferably C₁-C₄-alcoholates of metals of group 13, preferably Al(OCH(CH₃)₂)₃.

Dehydroxylation of compounds IIa to compounds I may be achieved by standard methods of organic chemistry as described in Hartwig et al., Tetrahedron 1983 (16), 2609-2645; Kirwan et al., and Tetrahedron 1990 (31), 5093-5096.

Dehydroxylation of compounds IIa to compounds II may comprise the reaction of compounds IIa with a metal hydride, or with a hydrosilane, preferably with a hydrosilane.

In one embodiment, compounds IIa are dehydroxylated by esterfication with a strong organic acid that is optionally dissolved in an inert solvent, followed by reaction with a hydrosilane. The process is usually carried out at temperatures of from 0 to 40° C., preferably from 15 to 35° C., in an aprotic solvent, in the presence of an organic acid.

Suitable aprotic solvents are aliphatic hydrocarbons, preferably C₅-C₁₆-alkanes, such as pentane, hexane, cyclohexane, and petrol ether; aromatic hydrocarbons, preferably C₆-C₁₂-aromatic hydrocarbons, such as toluene, o-, m-, and p-xylene; halogenated hydrocarbons, preferably halogenated C₁-C₆-alkanes and halogenated C₆-C₁₀-aromatic hydrocarbons, preferably halogenated C₁-C₆-alkanes and halogenated C₆-C₁₀-aromatic hydrocarbons, such as CH₂Cl₂, CHCl₃, and chlorobenzene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, dioxane, anisole, and THF; nitriles, preferably C₁-C₆-nitriles, such as CH₃CN, and propionitrile; ketones such as C₁-C₆-alkyl-C₁-C₆-alkyl ketones, C₁-C₆-alkyl-C₆-C₁₀-aryl ketones, and C₆-C₁₀-aryl-C₆-C₁₀-aryl-ketones, CH₃C(O)CH₃, CH₃C(O)CH₂CH₃, CH₃CH₂C(O)CH₂CH₃, and tert-butyl methyl ketone (MTBK); moreover dimethyl sulphoxide (DMSO), dimethyl formamide (DMF), and dimethylacetamide (DMA), preferably halogenated hydrocarbons, such as preferably halogenated C₁-C₆-alkanes and halogenated C₆-C₁₀-aromatic hydrocarbons, more preferably chlorinated hydrocarbons, and in particular CH₂Cl₂. It is also possible to use mixtures of the solvents mentioned. In one embodiment, no solvent is used.

Suitable acids are carboxylic acids (preferably C₁-C₁₀-carboxylic acids), such as formic acid, acetic acid and propionic acid, or halogenated carboxylic acids (preferably halogenated C₁-C₁₀-carboxylic acids), such as mono-, di-, and trifluoroacetic acid, mono-, di-, and trichloroacetic acid, or pentafluorobenzoic acid; preferably mono-, di-, and trifluoroacetic acid, more preferably trifluoroacetic acid. In one embodiment, the acid is used as a solvent.

The process may involve an activating agent and/or a coupling agent. Suitable activating agents are halogenating agents, which are usually selected from chlorinating agents and brominating agents, such as oxalylchloride, thionylchloride, phosphortri- and pentabromide, phorphortri- and pentachloride, preferably from thionylchloride and oxalylchloride. Suitable coupling agents are selected from carbodiimides, such as DCC (dicyclohexylcarbodiimide) and DIC (diisopropylcarbodiimide), benzotriazole derivatives, such as HATU (O-(7-azabenzotriazol-1-yl)-N,N,N′,N′-tetramethyluronium hexafluorophosphate), HBTU ((Obenzotriazol-1-yl)-N,N,N′,N′-tetramethyluronium hexafluorophosphate) and HCTU (1H-benzotriazolium-1-[bis(dimethylamino)methylene]-5-chloro tetrafluoroborate) and phosphonium-derived activators, such as BOP ((benzotriazol-1-yloxy)-tris(dimethylamino) phosphonium hexafluorophosphate), PyBOP ((benzotriazol-1-yloxy)-tripyrrolidinphosphonium hexafluorophosphate) and PyBrOP (bromotripyrrolidinphosphonium hexafluorophosphate). Generally, the activator is used in excess. Usually, no activating agent and/or coupling agent is necessary.

In one embodiment, the process is carried out in the presence of a base. Typical bases applied are organic bases, such as pyridine, 4-N,N-dimethylamino-pyridine, tetramethylene diamine, piperidine, diisopropylamine, morpholine, and triethylamine, preferably pyridine, 4-N,N-dimethylaminopyridine, and diisopropylamine. Usually, esterfication can be achieved without a base.

The resulting ester may then be reduced with a hydrosilane. This process is usually carried out at temperatures of from 0 to 80° C., preferably from 10 to 50° C., more preferably from 15 to 30° C., in an aprotic solvent.

Suitable aprotic solvents are aliphatic hydrocarbons, preferably C₅-C₁₆-alkanes, such as pentane, hexane, cyclohexane, and petrol ether; aromatic hydrocarbons, preferably C₆-C₁₀-aromatic hydrocarbons, such as toluene, o-, m-, and p-xylene; halogenated hydrocarbons, preferably halogenated C₁-C₆-alkanes or halogenated C₆-C₁₀-aromatic hydrocarbons, such as CH₂Cl₂, CHCl₃, and chlorobenzene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, dioxane, anisole, and THF; nitriles, preferably C₁-C₆-nitriles, such as CH₃CN, and propionitrile; ketones, preferably C₁-C₆-alkyl-C₁-C₆-alkyl ketones, C₁-C₆-alkyl-C₆-C₁₀-aryl ketones, and C₆-C₁₀-aryl-C₆-C₁₀-aryl ketones, such as CH₃C(O)CH₃, CH₃C(O)CH₂CH₃, CH₃CH₂C(O)CH₂CH₃, and MTBK. Preferred solvents are CH₃C(O)CH₃, CH₃CN, CHCl₃, CH₂Cl₂, CCl₄, 1,2-dichloro ethane, benzene, xylene, toluene, CH₃CH₂OCH₂CH₃, CH₃OCH₃, petroleum ether, C₅-C₁₂-alkanes, preferably CH₂Cl₂ and benzene, more preferably CH₂Cl₂. It is also possible to use mixtures of the solvents mentioned.

Suitable hydrosilanes are C₁-C₆-alkyl silanes, C₆-C₁₀-arylsilanes, and mixed (C₁-C₆-alkyl)(C₆-C₁₀-aryl) silanes, such as trimethylsilane, triethylsilane, diphenylsilane, diphenylmethylsilane, dimethylphenylsilane, phenyldimethylsilane, or polymethylhydrosiloxane. Preferred hydrosilanes are C₁-C₆-alkyl silanes, such as trimethylsilane and triethylsilane, and C₆-C₁₀-arylsilanes, such as triphenylsilane and diphenylsilane, more preferred trimethylsilane and triethylsilane, and in particular triethylsilane.

The conditions for the above type of reductions are known from WO2012/0209005.

Compounds I with R¹ being OH may be reacted with an amine NHR¹²R¹³ to the corresponding amide (as described in WO02015128358).

This process may be carried out in an inert solvent, in the presence of a base and by activation with an activating agent, or a coupling agent.

Suitable solvents are aliphatic hydrocarbons, preferably C₅-C₁₆-alkane, such as pentane, hexane, cyclohexane, and petrol ether; aromatic hydrocarbons, preferably C₆-C₁₀-aromatic hydrocarbons, such as toluene, o-, m-, and p-xylene; halogenated hydrocarbons, preferably halogenated C₁-C₆-alkanes and halogenated C₆-C₁₀-aromatic hydrocarbons, such as CH₂Cl₂, CHCl₃, and chlorobenzene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, dioxane, anisole, and THF; nitriles, preferably C₁-C₆-nitriles, such as CH₃CN, and propionitrile; ketones, preferably C₁-C₆-alkyl-C₁-C₆-alkyl ketones, such as CH₃C(O)CH₃, CH₃C(O)CH₂CH₃, CH₃CH₂C(O)CH₂CH₃, and MTBK; moreover DMSO, DMF, and DMA, preferably DMF. It is also possible to use mixtures of the solvents mentioned.

Suitable activating agents are halogenating agents, which are usually selected from chlorinating agents and brominating agents, such as oxalylchloride, thionylchloride, phosphortri- and pentabromide, phorphortri- and pentachloride, preferably from thionylchloride and oxalylchloride. Suitable coupling agents are well known and are for instance selected from carbodiimides, such as DCC (dicyclohexylcarbodiimide) and DIC (diisopropylcarbodiimide), benzotriazole derivatives, such as HATU (O-(7-azabenzotriazol-1-yl)-N,N,N′,N′-tetramethyluronium hexafluorophosphate), HBTU ((Obenzotriazol-1-yl)-N,N,N′,N′-tetramethyluronium hexafluorophosphate) and HCTU (1H-benzotriazolium-1-[bis(dimethylamino)methylene]-5-chloro tetrafluoroborate) and phosphonium-derived activators, such as BOP, PyBOP, and PyBrOP. Generally, the activator is used in excess.

As a further alternative, compounds I with R¹ being OR¹¹ and R¹¹ being not H (ester form) can also be directly converted to the corresponding amide. This process is usually carried out at temperatures from 20 to 80° C., preferably from 30 to 70° C., more preferably from 40 to 60° C., and in particular from 45 to 55° C., in the presence of a catalyst, such as a metalorganic compound. Such reactions have been described by Levin et al., Synthetic Communications, 1982, (12) 989-993.

Suitable solvents are aliphatic hydrocarbons, preferably C₅-C₁₆-alkane, such as pentane, hexane, cyclohexane, and petrol ether; aromatic hydrocarbons, preferably C₆-C₁₀-aromatic hydrocarbons, such as toluene, o-, m-, and p-xylene; halogenated hydrocarbons, preferably halogenated C₁-C₆-alkanes and halogenated C₆-C₁₀-aromatic hydrocarbons, such as CH₂Cl₂, CHCl₃, and chlorobenzene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, dioxane, anisole, and THF; nitriles, preferably C₁-C₆-nitriles, such as CH₃CN, and propionitrile; ketones, preferably C₁-C₆-alkyl-C₁-C₆-alkyl ketones, such as CH₃C(O)CH₃, CH₃C(O)CH₂CH₃, CH₃CH₂C(O)CH₂CH₃, and MTBK; alcohols, preferably C₁-C₄-alcohols, such as CH₃OH, CH₃CH₂OH, CH₃CH₂CH₂OH, CH₃CH(OH)CH₃, CH₃(CH₂)₃OH, and C(CH₃)₃OH; moreover DMSO, DMF, and DMA. Preferred solvents are CH₃C(O)CH₃, CH₃CN, CH₃NO₂, CHCl₃, CH₂Cl₂, CCl₄, CH₂ClCH₂Cl, benzene, toluene, xylene CH₃CH₂OCH₂CH₃, CH₃OCH₃, and C₅-C₁₂-alkanes, preferably CH₂Cl₂ and benzene, more preferably benzene. In one embodiment, the solvent is a C₁-C₆-alkyl-C₁-C₆-alkyl ether, C₁-C₆-alkyl-C₆-C₁₀-aryl ether, C₁-C₆-nitrile, halogenated C₁-C₆-alkane, halogenated C₆-C₁₀-aromatic hydrocarbon, C₅-C₁₆-alkane, or C₆-C₁₀-aromatic hydrocarbon. It is also possible to use mixtures of the solvents mentioned.

Suitable metalorganic compounds are metal alkyl (e.g. C₁-C₆-alkyl metal) or metal aryl compounds (e.g. C₆-C₁₀-aryl metal), preferably of Fe, Ti, Zr, Al, more preferably Al, such as Al(CH₃)₃, Al(CH₂CH₃)₂, Al(CH₂CH₂CH₃)₃, Al(CH(CH₃)₂)₃, Al(CH₂CH₂CH₂CH₃)₃, Al(CH(CH₃)(CH₂CH₃)₃, Al(C(CH₃)₃)₃, or Al(C₆H₅)₃, preferably Al(CH₃)₃.

Alternatively, compounds I, or compounds VI, wherein R¹ is OR¹¹, can be further reacted to compounds XIV-A, or compounds XV-A, as described below, which may then be converted to the amides XIV-A, and XV-A, wherein R¹ is NR¹¹R¹². It is thus possible to alter R¹ in compounds I, II, V, VI, XIV-A, or XV-A, by amidation, esterfication, hydrolysis, or a combination of these reactions, to introduce a suitable group R¹ that may be present in the final compounds XIV-A, or XV-A. Thus, R¹ may have the same meaning for all compounds I, II, V, VI, XIV-A, and XV-A, or may have a different meaning between said compounds, depending on the reaction step in which R¹ is altered.

Compounds II are produced by reaction of compounds V

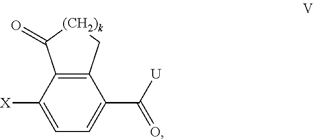

with H₂O, R¹¹OH, or NHR¹²R¹³; wherein X is halogen, and U is halogen; and wherein compounds V are produced by reaction of compounds III

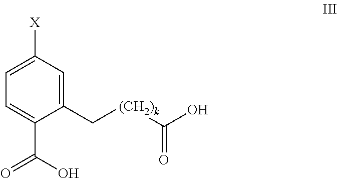

with a halogenating agent, followed by cyclization in the presence of a Lewis acid.

Compounds III are first activated by the conversion to an acid halogenide of formula IIIa

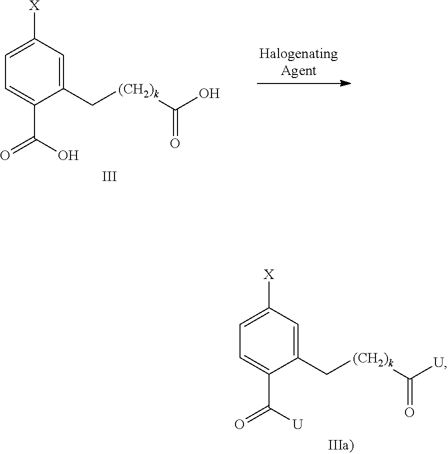

wherein each U is independently a halogen, preferably both U are the same halogen, and all other variables have a meaning as defined for compounds I. In one embodiment, U is Cl, Br, or I. In another embodiment, U is Br, or I. In yet another embodiment, U is Br. In yet another embodiment, U is Cl.

This process is usually carried out at temperatures of from 0 to 50° C., preferably from 10 to 30° C., in an inert solvent with a halogenating agent, optionally in the presence of a base.

Suitable inert solvents are aprotic solvents, such as aliphatic hydrocarbons, preferably C₁-C₁₆-alkanes, e.g. pentane, hexane, cyclohexane, and petrol ether; aromatic hydrocarbons, preferably C₆-C₁₀-aromatic hydrocarbons, e.g. toluene, o-, m-, and p-xylene; halogenated hydrocarbons, preferably halogenated aliphatic C₁-C₆-alkanes, or halogenated aromatic C₆-C₁₀-hydrocarbons, e.g. CH₂Cl₂, CHCl₃, and chlorobenzene; ethers, preferably C₁-C₆-alkyl-C₁-C₆-alkyl ethers and C₁-C₆-alkyl-C₆-C₁₀-aryl ethers, e.g. CH₃CH₂OCH₂CH₃, (CH₃)₂CHOCH(CH₃)₂, MTBE, DME, dioxane, anisole, and THF; nitriles, preferably C₁-C₆-nitriles e.g. CH₃CN, and propionitrile; ketones, preferably C₁-C₆-alkyl-C₁-C₆-alkyl ketones, e.g. CH₃C(O)CH₃, CH₃C(O)CH₂CH₃, CH₃CH₂C(O)CH₂CH₃, and MTBK; moreover DMSO, DMF, and DMA. Preferred solvents are DMF, CH₃C(O)CH₃, CHCl₃, CH₂Cl₂, CCl₄, benzene, toluene, xylene, 1,2-dichlorobenzene, CH₂ClCH₂Cl, CH₃CH₂OCH₂CH₃, CH₃OCH₃, petroleum ether, C₅-C₁₂-alkanes, preferably CH₂Cl₂, benzene and DMF, more preferably CH₂Cl₂ and DMF, most preferably CH₂Cl₂. It is also possible to use mixtures of the solvents mentioned. In one embodiment, the inert solvent is a mixture of CH₂Cl₂ and DMF, preferably with an excess of CH₂Cl₂. In one embodiment, the inert solvent is DMF, a halogenated aliphatic C₁-C₆-alkane, a halogenated aromatic C₆-C₁₀-hydrocarbon, a C₁-C₆-alkyl-C₁-C₆-alkyl ether, a C₁-C₆-alkyl-C₁-C₆-alkyl ketone, or a C₁-C₁₆-alkane. In one embodiment, the inert solvent is DMF, a halogenated hydrocarbon, an ether, a ketone, or an aliphatic hydrocarbon. In another embodiment, the inert solvent is DMF, or a halogenated hydrocarbon.

Suitable bases are, in general, organic bases, for example tertiary amines, such as trimethylamine, diisopropylethylamine, triethylamine, triisopropylethylamine and N-methylpiperidine, pyridine; substituted pyridines, such as collidine, lutidine and 4-dimethylaminopyridine; and also bicyclic amines. Particular preference is given to diisopropylethylamine. The bases are generally employed in catalytic amounts; however, they can also be used in equimolar amounts, in excess or, if appropriate, as solvent.

Halogenating agent are usually selected from chlorinating agents and brominating agents, such as oxalylchloride, thionylchloride, phosphortri- and pentabromide, phorphortri- and pentachloride, preferably from thionylchloride and oxalylchloride.

The ratio of chlorinating agent to compounds III usually depends on number of halogen atoms that can be transferred from the chlorinating agent. Usually, the halogenating agent (preferably the number of transferable halogen atoms) is applied in an excess of compounds III. The molar ratio of transferrable halogen atoms to the dicarboxylic acid III is usually from 1:1 to 20:1, more preferably from 2:1 to 15:1, and most preferably from 5:1 to 10:1.

Compounds IIIa are then cyclized to compounds V:

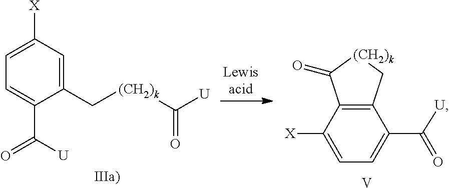

wherein X and U are independently halogen.

This process is usually carried out in the presence of Lewis acid in an inert solvent at low temperatures e.g. from −100 to 20° C., preferably from −80 to 10° C., more preferably from −20 to 5° C., which are then raised after mixture of the reactants to the boiling temperature of the solvent, e.g. to a range from 20 to 100° C., preferably from 25 to 50° C., more preferably from 30 to 50° C.

Suitable inert solvents are aliphatic hydrocarbons, preferably C₅-C₁₆-alkanes, such as pentane, hexane, cyclohexane, and petrol ether; aromatic hydrocarbons, preferably C₆-C₁₀-aromatic hydrocarbons, such as toluene, o-, m-, and p-xylene; halogenated hydrocarbons, preferably halogenated C₁-C₆-alkanes and halogenated C₆-C₁₀-aromatic hydrocarbons, such as CH₂Cl₂ CHCl₃, and chlorobenzene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, dioxane, anisole, and THF; nitriles, preferably C₁-C₆-nitriles, such as CH₃CN, and propionitrile; ketones, preferably C₁-C₆-alkyl-C₁-C₆-alkyl ketones, such as CH₃C(O)CH₃, CH₃C(O)CH₂CH₃, CH₃CH₂C(O)CH₂CH₃, and MTBK; moreover DMF, and DMA; preferably DMF, CH₃C(O)CH₃, CHCl₃, CH₂Cl₂, CCl₄, benzene, toluene, xylene, 1,2-dichlorobenzene, CH₂ClCH₂Cl, CH₃CH₂OCH₂CH₃, CH₃OCH₃, petroleum ether, or C₅-C₁₂-alkanes, more preferably CH₂Cl₂, benzene, or DMF, especially preferably CH₂Cl₂ or DMF, and in particular CH₂Cl₂. It is also possible to use mixtures of the solvents mentioned.

Lewis acids are protic acids, such as trifluoroacetic acid, CH₃SO₃H, or polyphosphoric acid; aprotic inorganic salts of metals of groups 13 or 14, and of transition metals of period 4, such as FeCl₃, FeBr₃, AlF₃, AlCl₃, AlBr₃, SbF₅, SbCl₅, BiF₃, BiCl₃, TiCl₄, ZnCl₂, SnCl₄, BF₃, BCl₃, BBr₃, ZrCl₄; or aprotic and metalorganic compounds of metals of groups 13 or 14, and of transition metals of period 4, such as Al(CH₃)₃, Al(CH₂CH₃)₃, B(CH₃)₃; moreover polyphosphate ester, and trimethylsilyl polyphosphate. Preferred Lewis acids are FeCl₃, FeBr₃, AlCl₃, and AlBr₃, more preferably AlCl₃.

Usually, an excess of a Lewis acid compared to compounds IIIa is applied, e.g. with a ratio from 1 to 5, preferably from 1 to 2, more preferably from 1.2 to 1.7 equivalents of the Lewis acid.

Cyclization reactions of the above described Friedel-Crafts-type are known, e.g. from England K. et al, Tetrahedron Letters, 2010 (51) 2849-2851, where a fluorine compound is used in ortho position to the carboxylic acid moiety of the phenyl ring. However, this would not lead to the desirable substitution pattern that is suitable for producing compounds I.

By the inventive method, compounds II are usually produced at high purity, although the cyclization could in theory also yield dimers, intramolecular anhydrides, and other side products.

Compounds V are then quenched with an alcohol R¹¹OH, or H₂O to yield compounds IIb, or with an amine NHR¹²R¹³ to yield compounds IIc

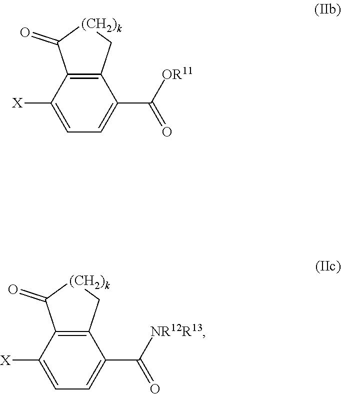

wherein all variables have a meaning as described form compounds I.

Compounds IIb with R¹¹ being not H can be hydrolyzed to the respective carboxylic acid. This process is usually carried out in the presence of a base or an acid in an inert solvent, and optionally H₂O.

Suitable inert solvents are aliphatic hydrocarbons, preferably C₅-C₁₆-alkanes, such as pentane, hexane, cyclohexane, and petrol ether; aromatic hydrocarbons, preferably C₆-C₁₀-aromatic hydrocarbons, such as toluene, o-, m-, and p-xylene; halogenated hydrocarbons, preferably halogenated C₁-C₆-alkanes and halogenated C₆-C₁₀-aromatic hydrocarbons, such as CH₂Cl₂, CHCl₃, and chlorobenzene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, dioxane, anisole, and THF; nitriles, preferably C₁-C₆-nitriles, such as CH₃CN, and propionitrile; ketones, preferably C₁-C₆-alkyl-C₁-C₆-alkyl ketones, such as CH₃C(O)CH₃, CH₃C(O)CH₂CH₃, CH₃CH₂C(O)CH₂CH₃, and MTBK; alcohols, preferably C₁-C₄-alcohols, such as CH₃OH, CH₃CH₂OH, CH₃CH₂CH₂OH, CH₃CH(OH)CH₃, CH₃(CH₂)₃OH, and C(CH₃)₃OH; moreover DMSO, DMF, and DMA. It is also possible to use mixtures of the solvents mentioned.

Suitable acids are mineral acids, such as hydrochloric acid, sulfuric acid, and organic acids, such as trifluoroacetic acid. Suitable bases are alkali metal hydroxides and earth alkali metal hydroxides, such as LiOH, NaOH or KOH.

Compounds IIb with R¹¹ being H may be reacted with an amine NHR¹²R¹³ to the compounds IIc (as described in WO02015128358).

This process may be carried out in an inert solvent, in the presence of a base and by activation with an activating agent, or a coupling agent.

Suitable inert solvents are aliphatic hydrocarbons, preferably C₅-C₁₆-alkane, such as pentane, hexane, cyclohexane, and petrol ether; aromatic hydrocarbons, preferably C₆-C₁₀-aromatic hydrocarbons, such as toluene, o-, m-, and p-xylene; halogenated hydrocarbons, preferably halogenated C₁-C₆-alkanes and halogenated C₆-C₁₀-aromatic hydrocarbons, such as CH₂Cl₂, CHCl₃, and chlorobenzene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, dioxane, anisole, and THF; nitriles, preferably C₁-C₆-nitriles, such as CH₃CN, and propionitrile; ketones, preferably C₁-C₆-alkyl-C₁-C₆-alkyl ketones, such as CH₃C(O)CH₃, CH₃C(O)CH₂CH₃, CH₃CH₂C(O)CH₂CH₃, and MTBK; moreover DMSO, DMF, and DMA, preferably DMF. It is also possible to use mixtures of the solvents mentioned.

Suitable activating agents are halogenating agents, which are usually selected from chlorinating agents and brominating agents, such as oxalylchloride, thionylchloride, phosphortri- and pentabromide, phorphortri- and pentachloride, preferably from thionylchloride and oxalylchloride. Suitable coupling agents are well known and are for instance selected from carbodiimides, such as DCC (dicyclohexylcarbodiimide) and DIC (diisopropylcarbodiimide), benzotriazole derivatives, such as HATU (O-(7-azabenzotriazol-1-yl)-N,N,N′,N′-tetramethyluronium hexafluorophosphate), HBTU ((Obenzotriazol-1-yl)-N,N,N′,N′-tetramethyluronium hexafluorophosphate) and HCTU (1H-benzotriazolium-1-[bis(dimethylamino)methylene]-5-chloro tetrafluoroborate) and phosphonium-derived activators, such as BOP, PyBOP, and PyBrOP. Generally, the activating agent, or coupling agent is used in excess.

As a further alternative, compounds IIb with R¹ being OR¹¹ and R¹¹ being not H (ester form) can also be directly converted to the corresponding amide, as described for compounds I above.

For illustration of the versatility of compounds V, they may alternatively be converted to compounds VII, VIII, or IX

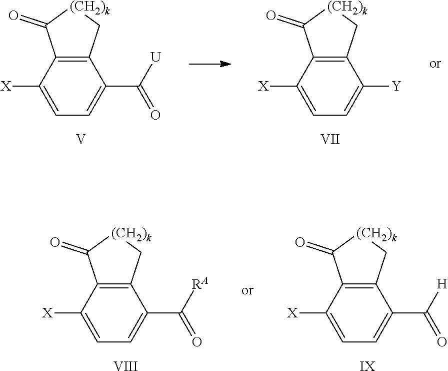

wherein X, Y, and U are each independently halogen, R^{A} has a meaning as defined for compounds I, and k is 1, or 2.

Typically compounds VII are produced by radical decarboxylation of compounds V, followed by halogenation. Such reactions are generally known as Hunsdiecker-Borodin reactions. This transformation is usually carried out in an inert solvent, in the presence of a radical formation agent, such as a Ag(I)halogenide, e.g. AgCl, AgBr, AgI, AgNO₃, AgSO₄, AgCO₃, and a halogen source such as halogen gas, or an inorganic halogenide, preferably an alkali halogenide, such as sodium chloride, sodium bromide, potassium chloride, potassium bromide. Alternatively, compounds V are reacted with compounds that are both a halogen source and a radical formation agent, such as N-bromsuccinimide, N-chlorsuccinimide, or selectfluor.

Suitable solvents are aliphatic hydrocarbons, preferably C₅-C₁₆-alkanes, such as pentane, hexane, cyclohexane, and petrol ether; aromatic hydrocarbons, preferably C₆-C₁₀-aromatic hydrocarbons, such as toluene, o-, m-, and p-xylene; halogenated hydrocarbons, preferably halogenated C₁-C₆-alkanes and halogenated C₆-C₁₀-aromatic hydrocarbons, such as methylene chloride, chloroform, and chlorobenzene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, dioxane, anisole, and tetrahydrofurane; nitriles, preferably C₁-C₆-nitriles, such as acetonitrile, and propionitrile; ketones, preferably C₁-C₆-alkyl-C₁-C₆-alkyl ketones, such as CH₃C(O)CH₃, CH₃C(O)CH₂CH₃, CH₃CH₂C(O)CH₂CH₃, and tert-butyl methyl ketone; alcohols, preferably C₁-C₄-alcohols, such as CH₃OH, CH₃CH₂OH, CH₃CH₂CH₂OH, CH₃CH(OH)CH₃, CH₃(CH₂)₃OH, and C(CH₃)₃OH; moreover DMSO, DMF, and DMA; preferably halogenated hydrocarbons, more preferably halogenated C₁-C₆-alkanes and halogenated C₆-C₁₀-aromatic hydrocarbons, such as methylene chloride, chloroform, and chlorobenzene, as well as nitriles such as acetonitrile, and propionitrile.

Compounds VII can also be produced by Curtius rearrangement of compounds V to the urethane, followed by hydrolysis to the amine, and followed by a Sandmeyer reaction, which are all reactions known to the skilled person and comprised in the above mentioned text books.

The Curtius rearrangement is usually carried out at temperatures of from 100 to 200° C., in an inert solvent, in the presence of an azide, e.g. sodium azide.

Hydrolysis is then usually carried out in the presence of a base or an acid in water. The Sandmeyer reaction usually requires reaction of the resulting amine with a nitrite, such as sodium nitrite, potassium nitrite or nitrous acid, in the presence of a halogenide, such as sodium halogenide, potassium halogenide, HF, HCl, or HBr.

Compounds VIII can be produced i.a. by reaction of compounds V with a Gillman reagent, or by conversion to a Weinreb-amide followed by reaction with a Grignard reagent, such as a metal-halogen-exchange reagent.

Suitable Gillman reagents are C₁-C₆-alkyl-Li compounds, C₁-C₆-alkyl-Cu compounds, or mixtures thereof. Examples of Gilman reagents are CH₃—Li or (CH₃)₂CuLi, CH₃CH₂—Li or (CH₃CH₂)₂CuLi, CH₃CH₂CH₂—Li or (CH₃CH₂CH₂)₂CuLi, (CH₃)₂CH—Li or (CH₃)₂CH CuLi, (CH₃)C—Li or (CH₃)C—CuLi, C₆H₆—Li or C₆H₆CuLi.

Suitable hydroxylamines for reaction with compounds V to the Weinreb-amide are hydroxylamine, N,O-dimethylamine, N,O-diethylamine, N,O-diisopropylamine, N,O-dipropylamine, N,O-dibutylamine, N,O-diisobutylamine, preferably N,O-dimethylamine.

Suitable Grignard reagents are C₁-C₆-alkyl metal and C₆-C₁₀-aryl metal compounds, such as C₁-C₆-alkyl lithium, C₆-C₁₀-aryl lithium, C₁-C₆-alkyl magnesium halogenide, C₆-C₁₀-aryl magnesium halogenide. Examples of Grignard reagents are, for example, C₆H₅—Li, CH₃—Li, CH₃CH₂—Li, CH₃(CH₂)₃—Li, (CH₃)₃C—Li, C₆H₅—MgCl, C₆H₅—MgCl, CH₃—MgCl, CH₃—MgBr, CH₃—MgI, CH₃CH₂—MgCl, CH₃CH₂—MgBr, CH₃CH₂—MgI, CH₃CH₂CH₂—MgCl, CH₃CH₂CH₂—MgBr, CH₃CH₂CH₂—MgI, (CH₃)₂CH—MgCl, (CH₃)₂CH—MgBr, (CH₃)₂CH—MgI, CH₃(CH₂)₃—MgCl, CH₃(CH₂)₃—MgBr, CH₃(CH₂)₃—MgI, CH₃CH(CH₂CH₃)—MgCl, CH₃CH(CH₂CH₃)—MgBr, CH₃CH(CH₂CH₃)—MgI, (CH₃)₃C—MgCl, (CH₃)₃C—MgBr, (CH₃)₃C—MgI, (CH₃)₂CHCH₂—MgCl, (CH₃)₂CHCH₂—MgBr, and (CH₃)₂CHCH₂—MgI.

Compounds IX can be produced i.a. by reaction of compounds V with hydrogen catalyzed by a Lindlar catalyst, or by conversion to the Weinreb amide, as described above, and subsequent reduction with an inorganic hydride as listed above, preferably with lithium aluminium hydride.

Compounds VII may be converted to compounds VIIb,

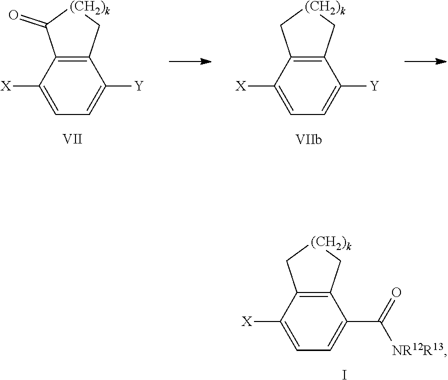

by processes as described above for the conversion of compounds II to compounds I, wherein X, and Y are independently from one another halogen, and k, R¹², and R¹³ have a meaning as defined for compounds I. Compounds VIIb may then again be further processed to compounds I with R¹ being NR¹²R¹³, by aminocarbonylation, as described in WO2008/145740.

Compounds I, IIb, IIc, VII, VIIb, VIII, and IX are precursors for a wide range of indane and tetralinyl derivatives. Examples of such derivatives are compounds Iaa to Ifd. The skilled person is able to devise suitable methods for the conversion of compounds IIb, IIc, VII, VIIb, VIII, and IX to compounds Iaa to Ifd by applying standard techniques of organic chemistry.

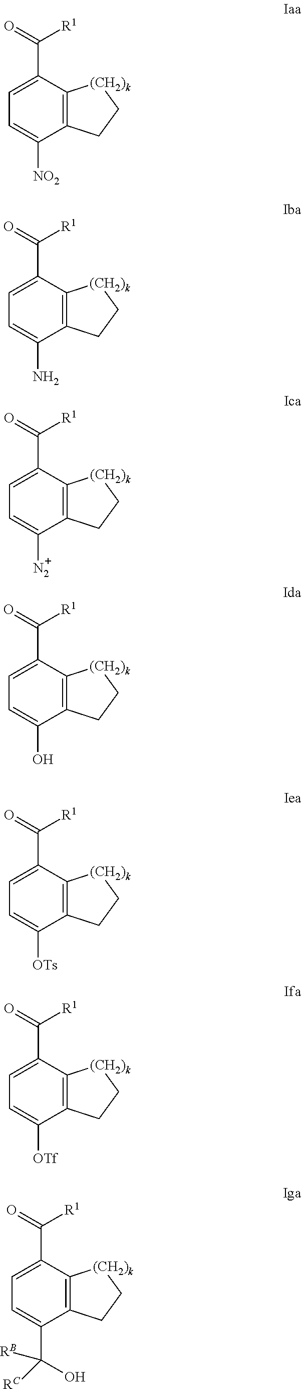

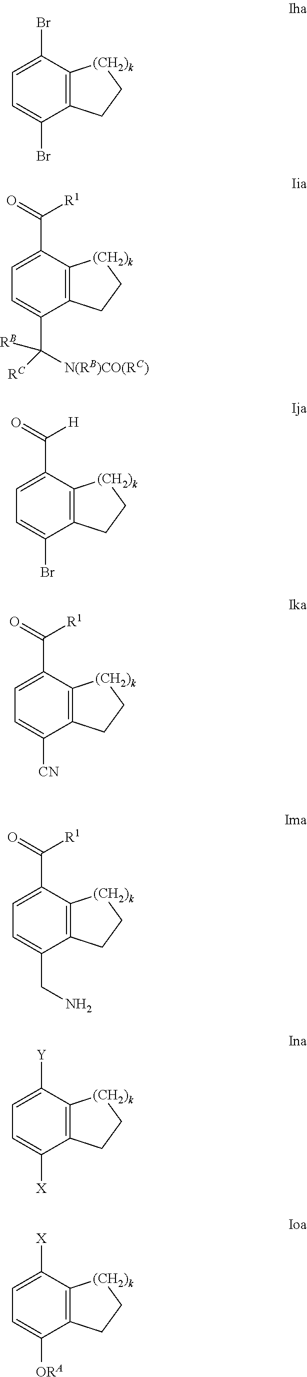

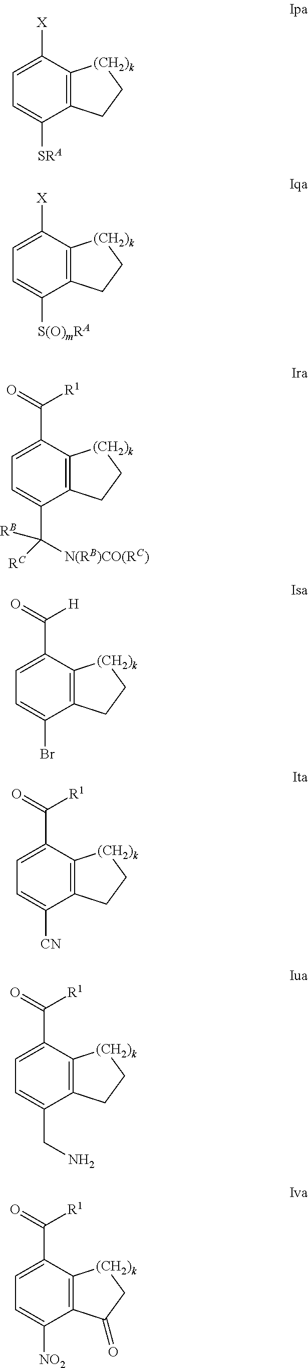

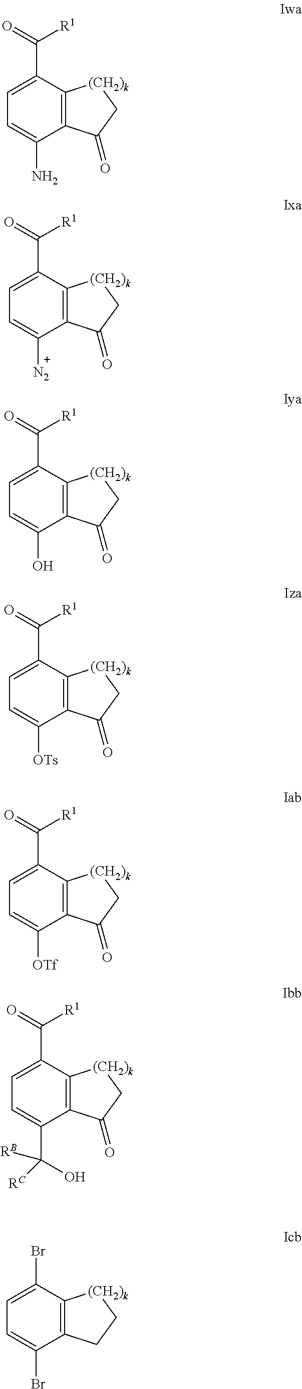

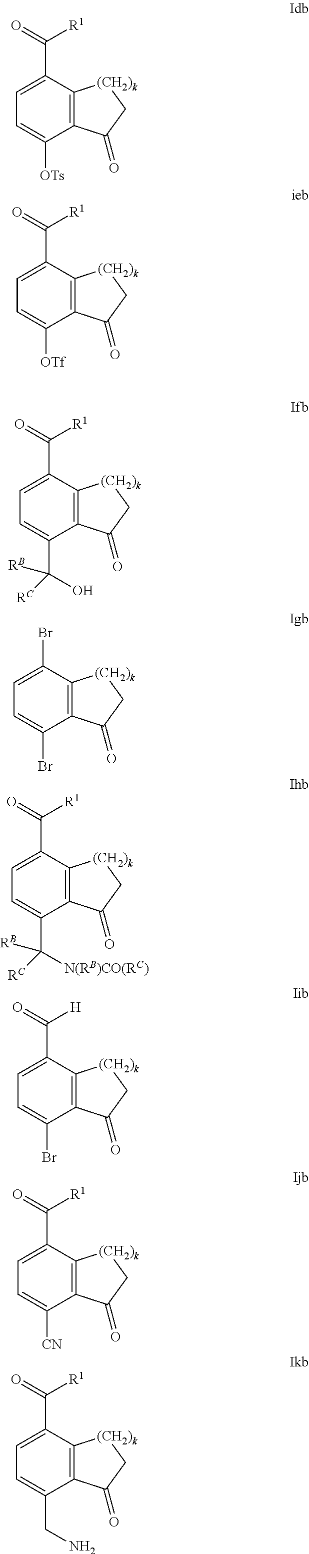

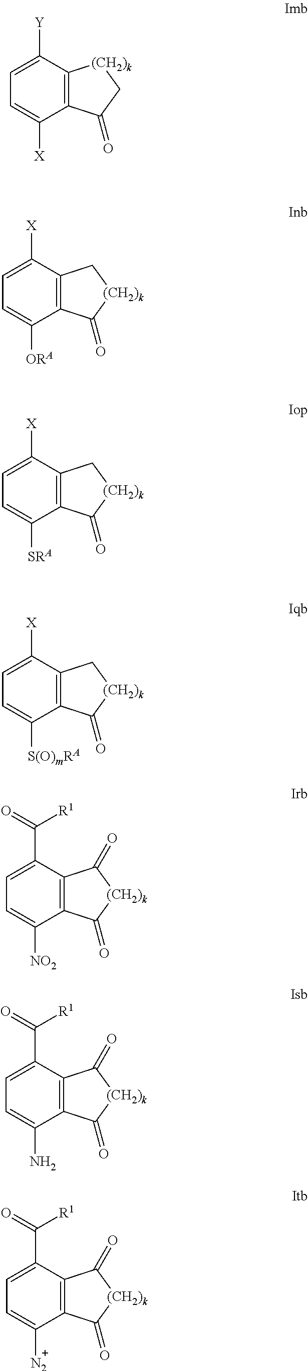

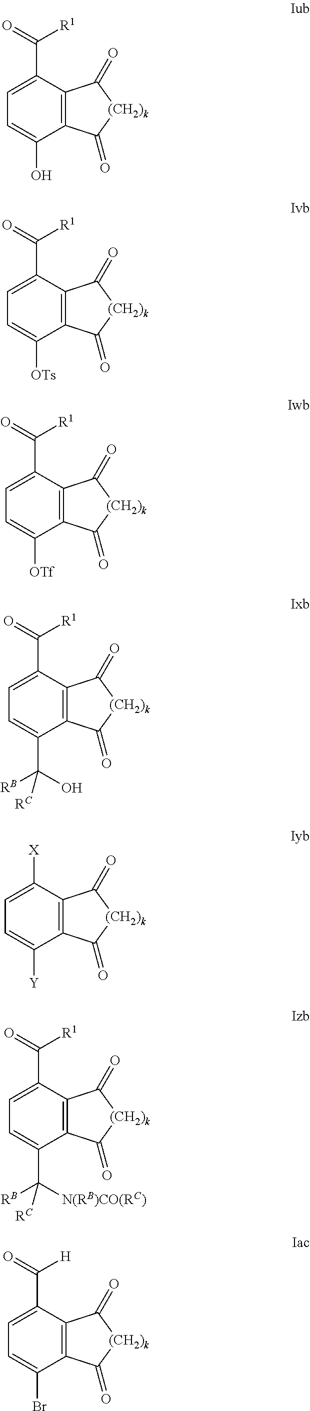

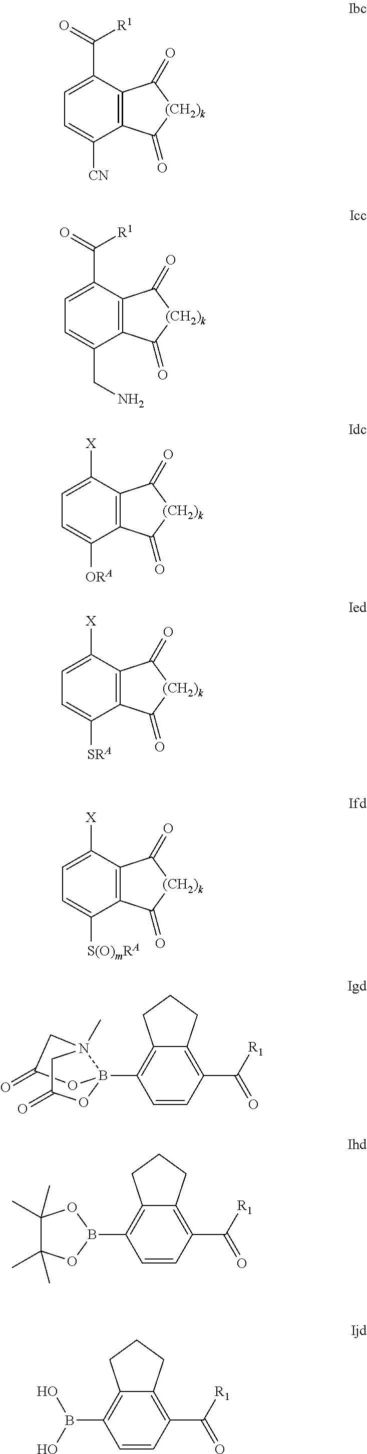

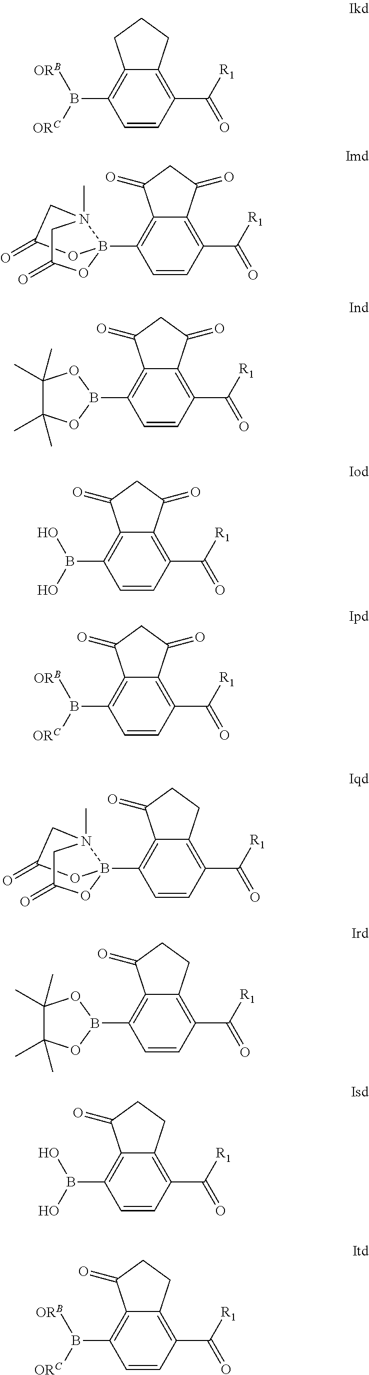

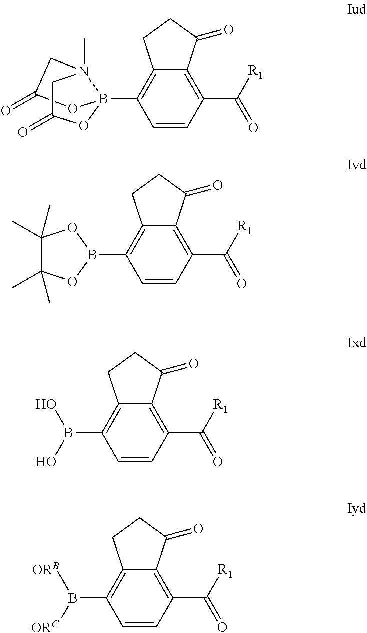

wherein all variables have a meaning as defined for compounds I.

Compounds III are produced by reaction of compounds IVa), IVb), or IVc) (collectively referred to as compounds IV) with hydrogen, followed by hydrolysis

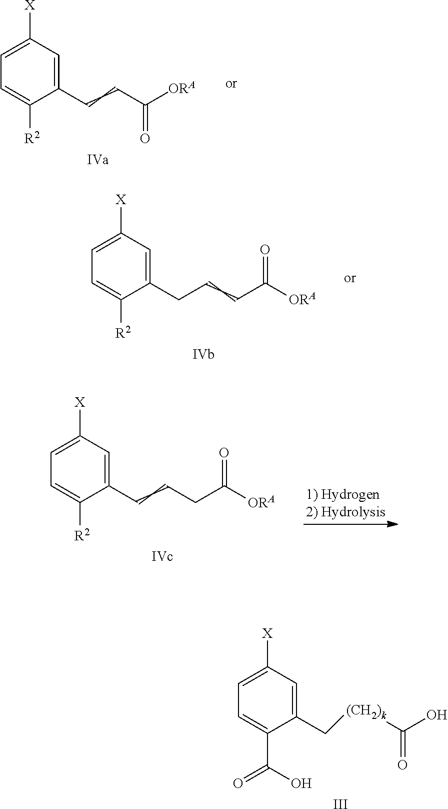

wherein R² is CN, or C(═O)OR^{A}, as defined for compounds I. The crossed bond in the above depicted structures IVa, IVb, and IVc relates to both an E- and a Z-configuration. The process usually involves either a reducing metal or hydrogen gas, and a base, such as an inorganic base, or an acid, such as a mineral acid. Preferably, the process involves a transition metal and an acid.

The term “reaction with hydrogen” may relate to hydrogen gas, which is introduced into the reaction mixture.

The process is usually carried out at a pressure from 1 to 100 psi, preferably 10 to 60 psi, in an inert solvent, in the presence of a catalyst.

Suitable inert solvents are aliphatic hydrocarbons, preferably C₅-C₁₆-alkanes, such as pentane, hexane, cyclohexane, and petrol ether; halogenated hydrocarbons, preferably halogenated C₁-C₆-alkanes and halogenated C₆-C₁₀-aromatic hydrocarbons, such as CH₂Cl₂, CHCl₃; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, dioxane, anisole, and THF; alcohols, preferably C₁-C₄-alcohols, such as CH₃OH, CH₃CH₂OH, CH₃CH₂CH₂OH, CH₃CH(OH)CH₃, CH₃(CH₂)₃OH, and C(CH₃)₃OH, preferably CH₃OH, CH₃CH₂OH, CH₃CH₂CH₂OH, CH₃CH(OH)CH₃, CH₃C(O)CH₃, CH₃OCH₃, CH₃CH₂OCH₂CH₃, MTBE, and THF, CHCl₃, CH₂Cl₂, CCl₄. It is also possible to use mixtures of the solvents mentioned.

Suitable catalysts are metals or their salts, preferably transition metals or their salts, which may be poisoned, e.g. by addition of sulfur-containing compounds. Preferred metals are platinum, palladium, nickel, as well as their salts and oxides, such as PtO₂, Pd on active coal, or Raney-nickel. Reactions of this type have been described in England K. et al, Tetrahedron Letters, 2010 (51) 2849-2851.

The term “reaction with hydrogen” may also relate to an embodiment, wherein compounds IVa to IVc are reacted with hydrogen gas and/or nascent hydrogen that is produced in situ.

Surprisingly, it has been found that processes according to this embodiment show a lower amount of side reactions, e.g. dehalogenation, compared to other methods, e.g. reaction with hydrogen gas in the presence of a catalyst, wherein the hydrogen is introduced into the reaction mixture.

This process is usually carried out at temperatures of from 70 to 90° C., more preferably from 75 to 85° C., more preferably from 78 to 82° C., in a protic solvent, in presence of a metal, and optionally at acidic pH, e.g. from 0 to 3.

The process may be carried out at temperatures of at least 65° C., preferably 75° C. The process may be carried out at temperatures up to 95° C., preferably up to 85° C.

Surprisingly, the reaction does not take place at temperatures below 60° C., or only with a reduced speed, therefore making it less economically effective. Furthermore, it was unexpectedly discovered that at temperatures above 95° C., major side reactions occur, thereby again making the process less economically effective.

On top, it was surprisingly found that this process does not lead to dehalogenation, which is a major side reaction under other reduction conditions, e.g. those involving hydrogen gas, which is introduced into the reaction mixture.

Suitable metals are selected from alkali metals, and alkaline earth metals, such as Li, Na, K, Rb, Cs, Mg, Ca, Sr, or Ba, metals of group 13, such as Al, and transition metals, such as Mn, Zn, Cr. In one embodiment, the metal is selected from alkali metals, alkaline earth metals, and transition metals.

Typical protic solvents are H₂O; C₁-C₄-alcohols, preferably CH₃OH, CH₃CH₂OH, CH₃CH₂CH₂OH, and CH₃CH(OH)CH₃; or an acid, preferably acetic acid, formic acid, HCl, H₂SO₄, or HNO₃. In one embodiment, the solvent is H₂O. In another embodiment, the solvent is acetic acid or HCl. In another embodiment, the solvent is an acid. It is also possible to use mixtures of the solvents mentioned.

Preferably, the metal has a standard electrode potential below 0 at a pH below 7.0. For clarification, the redox potential at a pH below 7.0 relates to a setup of half-cells, wherein all conditions except for the pH are the same as for the measurement of the common standard electron potential. Hence, a metal with a redox potential at a pH below 7.0 will be able to donate electrons to protons and thus produce hydrogen. Preferred metals are Li, Na, K, Zn, more preferably Zn.

It has furthermore surprisingly been found, that repeated addition of metal leads to economic advantages, such as an enhanced kinetic profile, or higher yields. Thus, in one embodiment, the metal is added repeatedly portionwise after several points of time, preferably once at the beginning and once after 3 to 20 hours, preferably 5 to 15 hours.

Preferably, the hydrogen gas and/or nascent hydrogen is produced in situ from either a metal selected from alkali metals and alkaline earth metals, or a metal with a redox potential below 0 at a pH below 7.0.

In one embodiment, the process is carried out at temperatures from 75 to 85° C., more preferably from 78 to 82° C., in a protic solvent, in presence of a Zn.

The resulting compounds IIIb

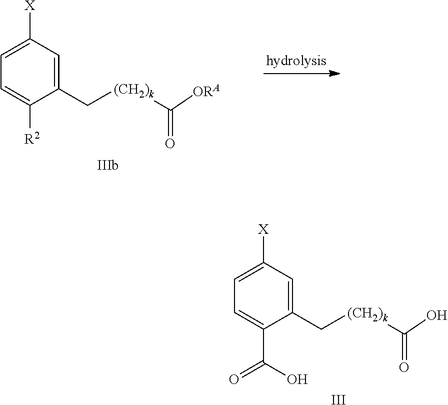

may then be hydrolyzed to compounds III by alkaline or acidic hydrolysis.

This transformation is usually carried out at temperatures of from 50 to 200° C., preferably from 80 to 150° C., more preferably from 100 to 150° C. in the presence of a base, or an acid.

Suitable bases are, in general, inorganic bases, such as alkali metal and alkaline earth metal hydroxides, such as LiOH, NaOH, KOH and Ca(OH)₂, alkali metal and alkaline earth metal oxides, such as lithium oxide, sodium oxide, calcium oxide, and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate; moreover organic bases, for example tertiary amines, such as trimethylamine, triethylamine, triisopropylethylamine and N-methylpiperidine, pyridine, substituted pyridines, such as collidine, lutidine and 4-dimethylaminopyridine, and also bicyclic amines.

Particular preference is given to inorganic bases, such as LiOH, NaOH, KOH, more preferably NaOH. The bases are generally employed in catalytic amounts; however, they can also be used in equimolar amounts, in excess or, if appropriate, as solvent.

Suitable acids and acidic catalysts are in general inorganic acids such as hydrofluoric acid, hydrochloric acid, hydrobromic acid, sulphuric acid und perchloric acid; Lewis acids, such as boron tri fluoride, aluminium tri chloride, iron-(III) chloride, tin-(IV) chloride, titanium-(IV) chloride and zinc-(II) chloride; moreover organic acids such as formic acid, acetic acid, propionic acid, oxalic acid, toluene sulphonic acid, benzene sulphonic acid, camphor sulphonic acid, citric acid, and trifluoro acetic acid. Particular preference is given to inorganic acids and organic acids, most preferably mineral acids and acetic acid, and in particular sulfuric acid, and acetic acid.

The acids are generally employed in catalytic amounts; however, they can also be used in equimolar amounts, in excess or, if appropriate, as solvent.

Compounds IVa, or IVc, may be produced by olefination of compounds Xa, and compounds IVb may be produced by olefination of compounds Xb,

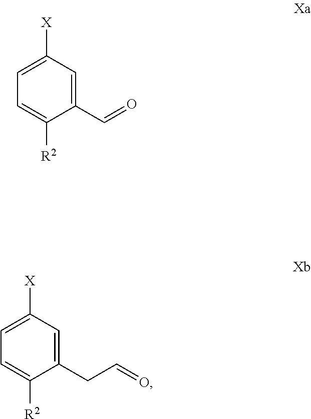

wherein X is halogen, and R² is CN or C(═O)OR^{A}, and R^{A} has a meaning as defined for compounds I. Typical olefination reactions include Peterson-olefinations, Wittig-reactions (as described in WO 2010/125130), Horner-Wadsworth-Emmons-reactions, or Julia-olefination, which are all known to the skilled person. Compounds IVa and IVc can be produced by olefination of compounds Xa, while compounds IVb can be produced by olefination of compounds Xb.

This process is usually carried out by reaction with a phosphine, or a phosphonate in the presence of a base at temperatures of −100 to 20° C., preferably from −80 to 10° C., more preferably from −50 to 0° C.

Suitable phosphonates are compounds of formula XIa

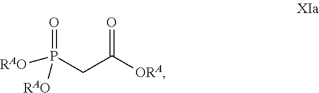

wherein each R^{A} may be independently selected and has a meaning as described for compounds I. Preferred phosphonates are di-C₁-C₆-alkylesters, di-C₆-C₁₀-arylesters, or mixed C₁-C₆-alkyl- and C₆-C₁₀-arylesters of phosphonates, e.g. dimethylphosphonates.

Suitable solvents are aliphatic hydrocarbons, preferably C₅-C₁₆-alkanes, such as pentane, hexane, cyclohexane, and petrol ether; aromatic hydrocarbons, preferably C₅-C₁₆-alkanes, such as toluene, o-, m-, and p-xylene, halogenated hydrocarbons, preferably halogenated C—C₆-alkanes and halogenated C₆-C₁₀-aromats, such as CH₂Cl₂, CHCl₃, and chlorobenzene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, dioxane, anisole, and THF. Preferred solvents are ethers, such DME. It is also possible to use mixtures of the solvents mentioned.

Suitable phosphines are compounds of formula XIb

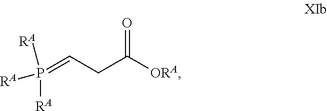

wherein each R^{A} may be independently selected and has a meaning as described for compounds I. Preferred phosphines are phosphines with R^{A} bound to phosphorous being C₁-C₆-alkyl, mixed C₁-C₆-alkyl and C₆-C₁₀-aryl, such as triphenylphosphines, trimethylphosphines, triisopropylphosphines, preferably triphenylphosphines.

Suitable bases are, in general, inorganic compounds, such as alkali metal and alkaline earth metal hydroxides, such as LiOH, NaOH, KOH and Ca(OH)₂; alkali metal and alkaline earth metal oxides, such as lithium oxide, sodium oxide, calcium oxide, and magnesium oxide; alkali metal and alkaline earth metal hydrides, such as lithium hydride, sodium hydride, potassium hydride and calcium hydride; alkali metal and alkaline earth metal carbonates, such as lithium carbonate, potassium carbonate and calcium carbonate, and also alkali metal bicarbonates, such as sodium bicarbonate; moreover organic bases, for example tertiary amines, such as trimethylamine, triethylamine, triisopropylethylamine and N-methylpiperidine, pyridine, substituted pyridines, such as collidine, lutidine and 4-dimethylaminopyridine, and also bicyclic amines; and metalorganic bases, such as n-butyl lithium, tert-butyl lithium, phenyl lithium, lithium diisopropylamide (LDA), or lithium bis(trimethylsilyl)amide. Particular preference is given to n-butyl lithium. The bases are generally employed in catalytic amounts; however, they can also be used in equimolar amounts, in excess or, if appropriate, as solvent.

Olefinations of the Horner-Wadsworth-Emmons-type are known from Li et al., Organic Letters 2013, (15) 6086-6089, Suppl. Inf.

Compounds XIa and XIb are either commercially available or can be produced from commercially available compounds by transesterfication, electrophilic substitution of a halogenide precursor etc.

The invention also relates to the production of compounds VI from compounds I,

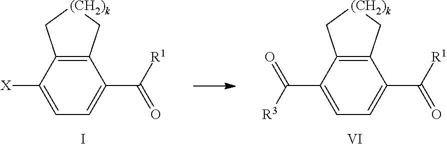

wherein R³ is H or C₃.

In case R³ is CH₃, compounds I are either reacted with a vinyl alcohol derivative, or magnesium, or a metal-halogen-transfer-reagent; and whereas in case R³ is H, compound V is either reacted with magnesium, or a metal-halogen-transfer-reagent, or with carbon monoxide.

In particular, in case R³ is CH₃, compounds I are
a) reacted with a vinyl alcohol derivative in the presence of a transition metal; or
b) they are reacted with magnesium or a metal-halogen-transfer-reagent, and an acetic acid derivative;
and in case R³ is H, compounds I are
c) reacted with carbon monoxide, and a reducing agent; or
d) they are reacted with magnesium, or a metal-halogen-transfer-reagent, and a formamide.

In case R³ is CH₃, compounds VI can be produced by either a cross-coupling reaction a), or by a Grignard reaction b) of compounds I.

Cross coupling reactions a) involve the reaction with a vinyl alcohol derivative.

Such cross coupling reactions are usually carried out in the presence of a catalyst, e.g. a transition metal such as Ni, Zn, Pd, Pt, preferably Pd, and a ligand L in an inert solvent by reaction with a vinyl alcohol derivative, and optionally in the presence of a base, preferably in the presence of a base. The transition metals are usually applied as salts, e.g. inorganic salts, or organic salts. In another embodiment, the cross coupling reactions are carried out in the presence of a base, a catalyst, e.g. a transition metal such as Ni, Zn, Pd, Pt, preferably Pd, and a ligand L in water by reaction with a vinyl alcohol derivative.

Suitable vinyl alcohol derivatives are vinyl alcohol, vinyl ethers, vinyl esters, vinyl amides, wherein the vinyl moiety may be substituted with a C₁-C₆-alkyl alkyl, C₁-C₆-haloalkyl, C₆-C₁₀-aryl, C₆-C₁₀-haloaryl, C₆-C₁₀-hetaryl, C₁-C₆-alkyl-C₆-C₁₀-aryl moiety. Preferred vinyl alcohol derivatives are vinyl alcohol, vinyl ethers and vinyl esters, such as vinyl alcohol, vinyl methyl ether, vinyl ethyl ether, vinyl isopropyl ether, vinyl n-propyl ether, vinyl n-butyl ether, vinyl tert-butyl ether, vinyl sec.-butyl ether, 1-vinyloxypropan-1-ol, 2-vinyloxypropan-1-ol, 3-vinyloxypropan-1-ol, 1-vinyloxypropan-2-ol, 2-vinyloxypropan-2-ol, 3-vinyloxypropan-2-ol, 1-vinyloxypropan-3-ol, 2-vinyloxypropan-3-ol, 3-vinyloxypropan-3-ol, vinylacetate, and vinyl propionate, preferably vinyl alcohol, vinyl methyl ether, vinyl n-butyl ether, 3-vinyloxypropan-1-ol, and vinyl acetate, more preferably vinyl alcohol, 3-vinyloxypropan-1-ol, vinyl acetate, most preferably 3-vinyloxypropan-1-ol. In another embodiment, the vinyl alcohol derivative is a vinyl ether, preferably a ethylene glycol vinyl ether.

Suitable inert solvents for the reaction are aprotic polar and non-polar solvents. Typical solvents are aliphatic hydrocarbons, preferably C₅-C₁₆-alkanes, such as pentane, hexane, cyclohexane, and petrol ether; aromatic hydrocarbons, preferably C₆-C₁₀-aromatic hydrocarbons, such as toluene, o-, m-, and p-xylene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, dioxane, anisole, and THF. The reaction is preferably carried out in aromatic hydrocarbons, (more preferably C₆-C₁₀-aromatic hydrocarbons), such as benzene, toluene, xylenes, cumene, or tert-butylbenzene, aprotic polar solvents, for example cyclic, or acyclic ethers, such as diethyl ether, tert-butyl methyl ether (MTBE), cyclopentyl methyl ether, THF or dioxane. In another embodiment, the reaction is carried out in water, thereby including aqueous mixtures with at least 10 wt % water with regard to the total mass of the reaction mixture.

Typical Pd(II)-salts, which are converted to the catalytically active Pd(0) complex during the reaction, are PdO, PdCl₂, PdBr₂, PdI₂, Pd(NO₃)₂, PdSO₄, or Pd(OAc)₂, preferably PdCl₂ or Pd(OAc)₂, more preferably Pd(OAc)₂.

Suitable ligands L are phosphine derivatives, such as preferably mono-, bi- or tridentate phosphine derivatives, e.g. triphenylphosphine, tricyclohexylphosphine, phosphinooxazolines, 2,2′-bis(diphenylphosphino)-1,1′-binaphthyl), 2,2′-bis(diphenylphosphino)-1,1′-binaphthyl, bis(diphenylphosphino) methane, bis(diphenylphosphino) ethane, diphenyl-2-pyridylphosphine, and 1,3-bis(di-iso-propylphosphino)propane, preferably triphenylphosphine, diphenyl-2-pyridylphosphine, and 1,3-bis(di-iso-propylphosphino)propane. In one embodiment, the ligand L is bis(diphenylphosphino) propane.

Suitable bases are inorganic bases and organic bases. Inorganic bases are usually alkali metal and alkaline earth metal hydroxides, such as LiOH, NaOH, KOH and Ca(OH)₂; alkali metal and alkaline earth metal oxides, such as lithium oxide, sodium oxide, calcium oxide, and magnesium oxide; alkali metal and alkaline earth metal hydrides, such as lithium hydride, sodium hydride, potassium hydride and calcium hydride; alkali metal and alkaline earth metal carbonates, such as lithium carbonate, potassium carbonate and calcium carbonate; alkali metal bicarbonates, such as sodium bicarbonate; and silver hydroxides or silver carbonates. Organic bases are usually tertiary amines, such as trimethylamine, triethylamine, 4-N,N-dimethylaminopyridine, triisopropylethylamine and N-methylpiperidine, pyridine, substituted pyridines, such as collidine, lutidine and 4-dimethylamino-pyridine, and also bicyclic amines; alkali metal acetates, such as NaOAc, KOAc; moreover secondary amines, such as, tetramethylethylendiamine, tetramethylene diamine, piperidine, diisopropylamine, morpholine, preferably pyridine, 4-N,N-dimethylaminopyridine, diisopropylamine, Na₂CO₃, K₂CO₃, NaOAc, KOAc, Ag₂CO₃ and triethylamine, and most preferably K₂CO₃.

Typical cross-coupling reactions are Heck reactions, Stille reactions, Suzuki reactions, Negishi reaction, and Kumada reactions, which are known to the skilled person, also including typical reaction parameters, reactants etc, and which may be found in Metal-Catalyzed Cross-Coupling Reactions and More, de Meijere A., Wiley VCH, 2014.

In one embodiment compounds VI are produced by the following Heck reaction of compounds I with compounds XII-A

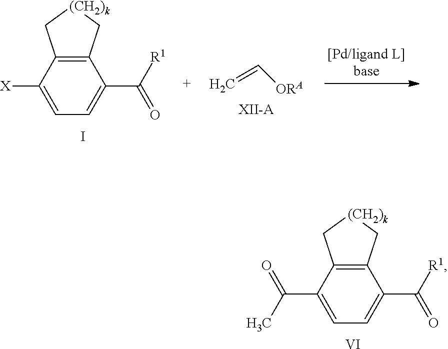

wherein R^{A} has the same meaning as defined for compounds I, preferably C₁-C₆-alkyl, which may be partially or fully substituted by OH, preferably C₁-C₄-alkyl, which may be partially substituted by OH, more preferably CH₂CH₃, CH₂CH₂CH₃, CH₂CH₂CH₂CH₃, or CH₂CH₂OH, and in particular CH₂CH₂OH.

The temperature for such cross coupling reactions is typically from 70 to 120° C., preferably from 80 to 110° C., most preferably from 85 to 95° C. In one embodiment, the temperature is at least 60° C. In another embodiment, the temperature is at most 110° C.

In another embodiment compounds VI with R³ being CH₃ are produced by the following Stille reaction of compounds I with compounds XII-B

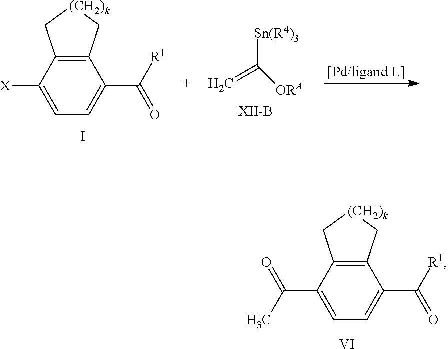

wherein R^{A} has the same meaning as defined for compounds I, preferably methyl or ethyl; and wherein each R⁴ is independently a C₁-C₆ alkyl, C₁-C₆-cycloalkyl, or phenyl, preferably methyl, butyl, and phenyl, more preferably butyl.

Compounds VI with R³ being CH₃ may also be produced via a Grignard reaction of compounds I:

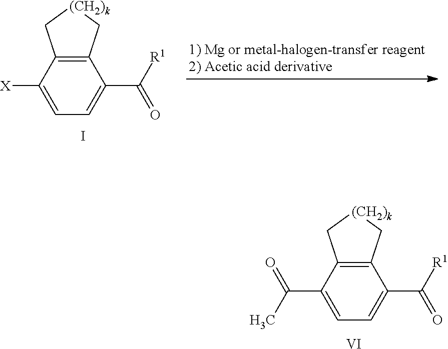

This transformation is usually carried an aprotic polar or non-polar solvent in the presence of Mg, or a metal-halogen-transfer reagent, as well as of an acetic acid derivative, in a two-step process.

The reaction is preferably carried out at temperatures of from −78 to 110° C. In general, the upper temperature limits the boiling point of the solvent in question when the reaction is carried out under atmospheric pressure. The first step of the reaction is preferably carried out at temperatures of −30 to 110° C. The second step (electrophile addition) is preferably carried out at temperatures of −78 to 50° C.

Suitable solvents for the reaction are aprotic polar and non-polar solvents. Typical solvents are aliphatic hydrocarbons, preferably C₅-C₁₆-alkanes, such as pentane, hexane, cyclohexane, and petrol ether; aromatic hydrocarbons, preferably C₆-C₁₀-aromatic hydrocarbons, such as toluene, o-, m-, and p-xylene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, dioxane, anisole, and THF. The reaction is preferably carried out in aromatic hydrocarbons, preferably C₆-C₁₀-aromatic hydrocarbons, such as benzene, toluene, xylenes, cumene, chlorobenzene, nitrobenzene, or tert-butylbenzene, aprotic polar solvents, for example cyclic, or acyclic ethers, such as diethyl ether, tert-butyl methyl ether (MTBE), cyclopentyl methyl ether, THF or dioxane.

In case the reaction is carried out in the presence of magnesium, the magnesium can be activated by halogens organohalogenides, such as like iodine, bromine, dibromo ethane, monobromo ethane.

Metal-halogen-transfer reagents suitable for the reaction are C₁-C₆-alkyl metal and C₆-C₁₀-aryl metal compounds, such as C₁-C₆-alkyl lithium, aryl lithium, C₁-C₆-alkyl magnesium halogenide, C₆-C₁₀-aryl magnesium halogenide. In one embodiment, the metal-halogen-transfer reagent is C₁-C₆-alkyl lithium, or aryl lithium. Examples of metal-halogen-transfer reagents are, for example, C₆H₅—Li, CH₃—Li, CH₃CH₂—Li, CH₃(CH₂)₃—Li, (CH₃)₃C—Li, C₆H₅—MgCl, C₆H₅—MgCl, CH₃—MgCl, CH₃—MgBr, CH₃—MgI, CH₃CH₂—MgCl, CH₃CH₂—MgBr, CH₃CH₂—MgI, CH₃CH₂CH₂—MgCl, CH₃CH₂CH₂—MgBr, CH₃CH₂CH₂—MgI, (CH₃)₂CH—MgCl, (CH₃)₂CH—MgBr, (CH₃)₂CH—MgI, CH₃(CH₂)₃—MgCl, CH₃(CH₂)₃—MgBr, CH₃(CH₂)₃—MgI, CH₃CH(CH₂CH₃)—MgCl, CH₃CH(CH₂CH₃)—MgBr, CH₃CH(CH₂CH₃)—MgI, (CH₃)₃C—MgCl, (CH₃)₃C—MgBr, (CH₃)₃C—MgI, (CH₃)₂CHCH₂—MgCl, (CH₃)₂CHCH₂—MgBr, and (CH₃)₂CHCH₂—MgI.

The reaction is preferably carried out with CH₃—MgCl, CH₃—MgBr, CH₃CH₂—MgCl, CH₃CH₂—MgBr, CH₃CH₂CH₂—MgCl, CH₃CH₂CH₂—MgBr, (CH₃)₂CH—MgCl, (CH₃)₂CH—MgBr, CH₃(CH₂)₃—MgCl, CH₃(CH₂)₃—Br, CH₃CH(CH₂CH₃)—MgCl, CH₃CH(CH₂CH₃)—MgBr, (CH₃)₃C—MgCl, (CH₃)₃C—MgBr, (CH₃)₂CHCH₂—MgCl, or (CH₃)₂CHCH₂—MgBr.

The reaction is more preferably carried out with CH₃—MgCl, CH₃—MgBr, CH₃CH₂—MgCl, CH₃CH₂—MgBr, CH₃CH₂CH₂—MgCl, CH₃CH₂CH₂—MgBr, (CH₃)₂CH—MgCl, (CH₃)₂CH—MgBr, CH₃(CH₂)₃—MgCl, or CH₃(CH₂)₃—Br,

The reaction is most preferably carried out with CH₃—MgCl, CH₃—MgBr, (CH₃)₂CH—MgCl, (CH₃)₂CH—MgBr, (CH₃)₃C—MgCl, or (CH₃)₃C—MgBr, and in particular with (CH₃)₂CH—MgCl, or (CH₃)₂CH—MgBr.

In case the reaction is carried out with a C₁-C₆-alkyl magnesium halogenide, or a C₆-C₁₀-aryl magnesium halogenide, a lithium salt may be added, preferably LiCl.

The acetic acid derivative may be an acetic acid ester (e.g. methyl, ethyl, propyl, isopropyl, n-butyl, isobutyl, tert-butyl, phenyl ester), an acetic acid halogenide, e.g. chloride, acetic acid bromide, or CH₃CN, preferably an acetic acid ester or acetic acid chloride, more preferably methyl acetate, ethyl acetate or acetic acid chloride, most preferably acetic acid chloride.

The magnesium, or the metal-halogen-transfer reagent is preferably employed in a molar ratio of from 0.9 to 2 mol per mol of the compounds I. Preferably, from 0.9 to 1.2 mol, in particular from about 0.95 to 1.1 mol, of magnesium or a metal-halogen-transfer reagent are employed per mol of compounds I.

The acetic acid derivatives are preferably employed in a molar ratio of from 0.9 to 5 mol per mol of compounds I. Preferably, from 0.9 to 2 mol, in particular from about 0.95 to 1.5 mol, of acetic acid derivatives are employed per mol of the compounds I.

Compounds VI with R³ being H may be produced form compounds I by various methods, which were generally described in Natte et. al., Angewandte Chemie, 2014, (53) 10090-10094; Neumann et al, Chemistry Asian Journal, 2012, (7) 2213-2216; Ashfield et al, Organic Process Research and Development, 2007, (11) 39-43; Pétrier et al., Tetrahedron Letters, 1982, (23) 3361-3364, and Jiang et al., Journal of Chemical Research, 2014, (38) 218-222, such as reductive carbonylation a) or the Bouveault-aldehyde formation b).

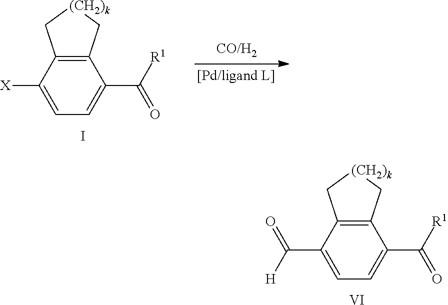

Reductive carbonylation c) is carried out by reaction with carbon monoxide (CO).

Usually, reductive carbonylation is carried out in the presence of a metal, preferably a transition metal, a ligand L, carbon monoxide, as well as a reducing agent.

Typical reducing agents are H₂, formic acid or its salts. The total gas pressure is usually from 1 to 20 bar, preferably from 2 to 15 bar, more preferably from 2 to 7 bar and the temperature is from 50 to 150° C., preferably from 70 to 130° C.

For example, compounds VI may be produced by reductive carbonylation of compounds I with carbon monoxide (CO) and hydrogen (H₂) by palladium catalysis.

Suitable solvents are aliphatic hydrocarbons, preferably C₅-C₁₆-alkanes, such as pentane, hexane, cyclohexane, and petrol ether; aromatic hydrocarbons, preferably C₆-C₁₀-aromatic hydrocarbons, such as toluene, o-, m-, and p-xylene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, dioxane, anisole, and THF; nitriles, preferably C₁-C₆-nitriles, such as CH₃CN, and propionitrile; alcohols, preferably C₁-C₄-alcohols, such as CH₃OH, CH₃CH₂OH, CH₃CH₂CH₂OH, CH₃CH(OH)CH₃, CH₃(CH₂)₃OH, and C(CH₃)₃OH; preferably aromatic solvents such as benzene or toluene. It is also possible to use mixtures of the solvents mentioned.

Suitable gas ratios of CO to H₂ are from 40:60 to 60:40, preferably 50:50. The gas pressure may be from 1 to 20 bar, preferably 1 to 10 bar. Suitable Pd(II)-salts and ligands L are those described for the cross couplings above.

Bouveault-aldehyde formation d) usually involves the reaction with magnesium, or a metal-halogen transfer agent, as described above in an inert solvent, and the subsequent reaction with a formamide.

Suitable formamides are formamide and N-akylformamides, such as methylformamide, DMF, phenylformamide.

Suitable inert solvents are aliphatic hydrocarbons, preferably C₅-C₁₆-alkanes, such as pentane, hexane, cyclohexane, and petrol ether; aromatic hydrocarbons, preferably C₆-C₁₀-aromatic hydrocarbons, such as toluene, o-, m-, and p-xylene; halogenated hydrocarbons, preferably halogenated C₁-C₆-alkanes and halogenated C₆-C₁₀-aromatic hydrocarbons, such as CH₂Cl₂, CHCl₃, and chlorobenzene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, dioxane, anisole, and THF; preferably C₅-C₁₆-alkanes, or C₆-C₁₀-aromatic hydrocarbons.

It is also possible to use mixtures of the solvents mentioned.

Compounds I, or compounds VI are intermediates for the manufacture of the insecticidal compounds XIV

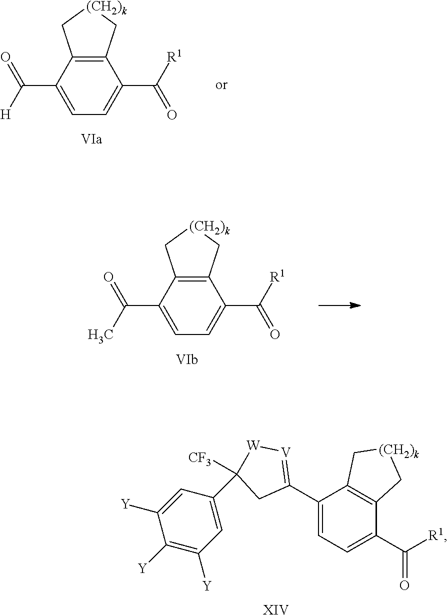

wherein each Y is independently a halogen, V is selected from CH and N, W is selected from O, S and CH₂, and R¹ and k have a meaning as defined for compounds I.

In one embodiment, compounds I, or compounds VI are intermediates for the manufacture of insecticidal compounds XIV-A

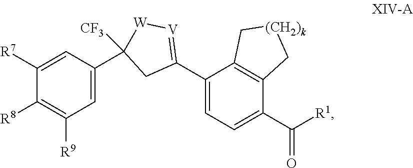

wherein V is selected from CH, N, and NO, W is selected from O, S and CH₂, R¹ and k have a meaning as defined for compounds I, and R⁷, R⁸, and R⁹ are independently hydrogen, halogen, halomethyl, or halomethoxy, wherein at most two substituents R⁷, R⁸, and R⁹ are H.

In yet another embodiment, compounds I and VI are intermediates for the manufacture of insecticidal compounds XIV-B

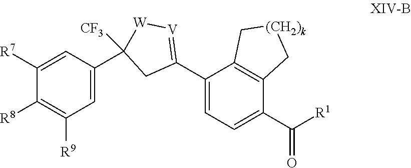

wherein R⁷, R⁸, and R⁹ correspond to a line A-1 to A-31 of Table 1:

TABLE 1

| No. | R⁷ | R⁸ | R⁹ |
|---|---|---|---|
| A-1 | F | H | F |
| A-2 | F | F | F |
| A-3 | F | Cl | F |
| A-4 | F | Br | F |
| A-5 | F | H | Cl |
| A-6 | F | H | Br |
| A-7 | Cl | H | Cl |
| A-8 | Cl | Cl | Cl |
| A-9 | Cl | F | Cl |
| A-10 | Cl | Br | Cl |
| A-11 | Cl | H | Br |
| A-12 | Br | H | Br |
| A-13 | Br | F | Br |
| A-14 | Br | Cl | Br |
| A-15 | CF₃ | H | F |
| A-16 | CF₃ | H | Cl |
| A-17 | CF₃ | H | Br |
| A-18 | CF₃ | H | CF₃ |
| A-19 | CF₃ | F | F |
| A-20 | CF₃ | Cl | Cl |
| A-21 | CF₃ | Br | Br |
| A-22 | OCF₃ | H | F |
| A-23 | OCF₃ | H | Cl |
| A-24 | OCF₃ | H | Br |
| A-25 | OCF₃ | H | CF₃ |
| A-26 | OCF₃ | H | H |
| A-27 | CF₃ | H | H |
| A-28 | Br | H | H |
| A-29 | Cl | H | H |
| A-30 | F | H | H |
| A-31 | Cl | F | H |

In one embodiment, compounds of lines A-19, A-22, A-23, and A-24 are manufactured from compounds I and VI. Compounds XV, and XV-B, fall under the definition of compounds XV-A and are thus preferred embodiments of XV-A for all compounds and reactions disclosed herein.

In one embodiment, insecticidal compounds XIV-A that are manufactured from compounds I, or compounds VI are selected from

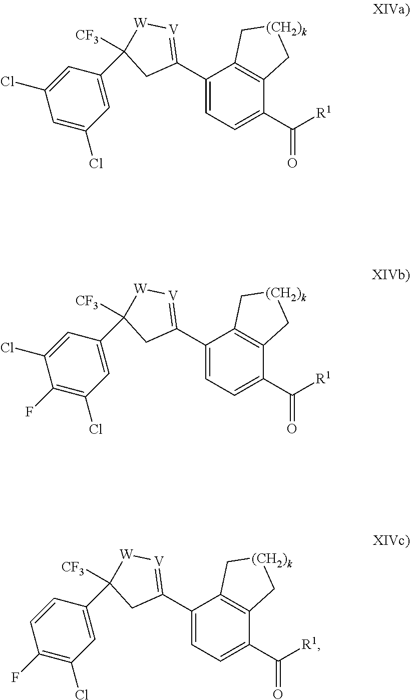

wherein all variables have a meaning as defined for compounds XIV.

In another embodiment, insecticidal compounds XIV-A that are manufactured from compounds I, or compounds VI are selected from

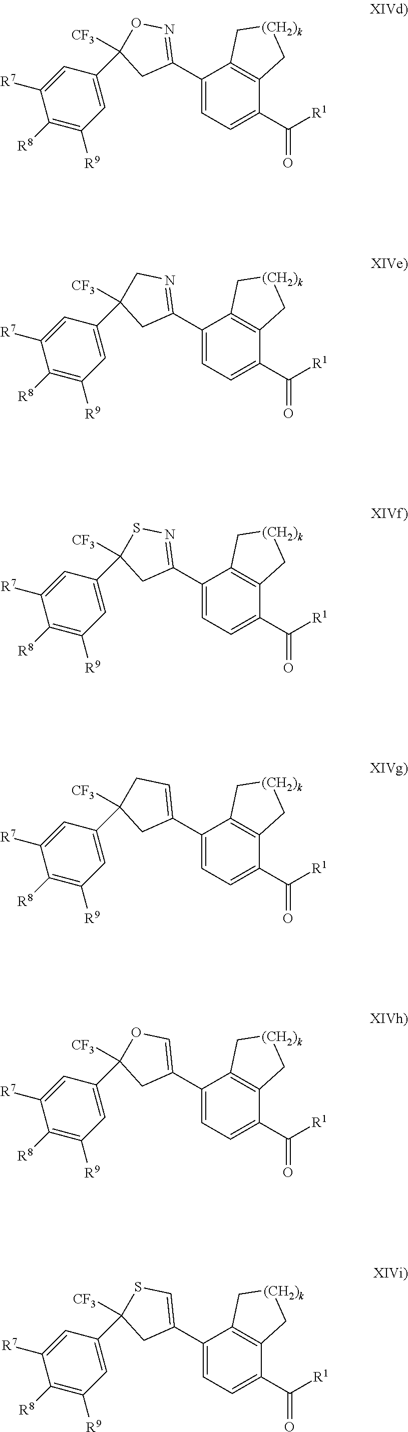

where all variables have a meaning as defined for compounds XIV-A. In one embodiment, R⁷, R⁸, and R⁹ are independently selected from halogen.

In yet another embodiment, insecticidal compounds XIV-A are selected from compounds XIVd, XIVe, XIVf, and XIVg. In yet another embodiment, insecticidal compounds XIV-A are selected from compounds XIVd to XIVi. In yet another embodiment, insecticidal compounds XIV-A are compounds XIVj. In yet another embodiment, insecticidal compounds XIV-A are compounds XIVh.

Suitable manufacture processes to yield insecticidal compounds XIV-A from compounds VI are disclosed in WO2010/125130 and WO2015128358, WO2014206908, EP2172462, and WO02014206910.

The process for the production of compounds XIV-A from compounds I, or compounds VI may involve the condensation of compounds I, or compounds VI, with acetophenone compounds XVIII-A to compounds XIX.

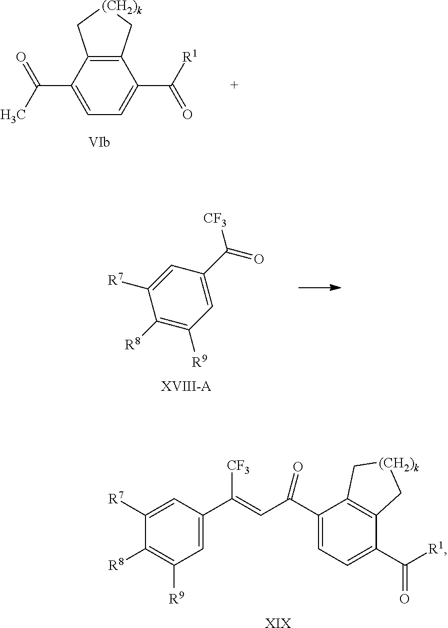

where all the variables have a meaning as defined for compounds XIV-A.

This transformation is usually carried out at temperatures of from 100 to 140° C., preferably from 110 to 130° C., in an inert solvent, in the presence of a base.

Suitable solvents are aliphatic hydrocarbons such as pentane, hexane, cyclohexane, and petrol ether; aromatic hydrocarbons such as toluene, o-, m-, and p-xylene; halogenated hydrocarbons such as methylene chloride, dichloroethane, chloroform, and chlorobenzene; ethers such as diethylether, diisopropylether, tert-butylmethylether, dioxane, anisole, and tetrahydrofurane; nitriles such as acetonitrile, and propionitrile; ketones such as acetone, methyl ethyl ketone, diethyl ketone, and tert-butyl methyl ketone; alcohols such as CH₃OH, CH₃CH₂OH, CH₃CH₂CH₂OH, CH₃CH(OH)CH₃, CH₃(CH₂)₃OH, and C(CH₃)₃OH; moreover DMSO, DMF, and DMA; preferably halogenated hydrocarbons, in particular dichloroethane. It is also possible to use mixtures of the solvents mentioned.

Suitable bases are, in general, inorganic compounds, such as alkali metal and alkaline earth metal hydroxides, such as LiOH, NaOH, KOH and Ca(OH)₂; alkali metal and alkaline earth metal oxides, such as lithium oxide, sodium oxide, calcium oxide, and magnesium oxide; alkali metal and alkaline earth metal hydrides, such as lithium hydride, sodium hydride, potassium hydride and calcium hydride; alkali metal and alkaline earth metal carbonates, such as lithium carbonate, potassium carbonate and calcium carbonate; and also alkali metal bicarbonates, such as sodium bicarbonate; moreover organic bases, for example tertiary amines, such as trimethylamine, triethylamine, triisopropylethylamine and N-methylpiperidine, pyridine; substituted pyridines, such as collidine, lutidine and 4-dimethylaminopyridine; and also bicyclic amines. Particular preference is given to alkali metal and alkaline earth metal carbonates, as well as tertiary amines, in particular K₂CO₃, and triethylamine. The bases are generally employed in catalytic amounts; however, they can also be used in equimolar amounts, in excess or, if appropriate, as solvent. It is also possible to use mixtures of the bases mentioned.

The starting materials are generally reacted with one another in equimolar amounts. In terms of yield, it may be advantageous to employ an excess of XIII, based on Vib).

Compounds XIX may then be reacted with hydroxylamine to the isoxazoline compounds XIV-A, wherein V is N, and W is O. This transformation is usually carried out at temperatures of from 10 to 50° C., preferably from 20 to 30° C., in an inert solvent, in the presence of a catalyst, and a base.

Suitable solvents are aliphatic hydrocarbons such as pentane, hexane, cyclohexane, and petrol ether; aromatic hydrocarbons such as toluene, o-, m-, and p-xylene; halogenated hydrocarbons such as methylene chloride, dichloroethane, chloroform, and chlorobenzene; ethers such as diethylether, diisopropylether, tert-butylmethylether, dioxane, anisole, and tetrahydrofurane; nitriles such as acetonitrile, and propionitrile; ketones such as acetone, methyl ethyl ketone, diethyl ketone, and tert-butyl methyl ketone; alcohols such as CH₃OH, CH₃CH₂OH, CH₃CH₂CH₂OH, CH₃CH(OH)CH₃, CH₃(CH₂)₃OH, and C(CH₃)₃OH; moreover DMSO, DMF, and DMA; preferably halogenated hydrocarbons, in particular dichloroethane. It is also possible to use mixtures of the solvents mentioned.

Suitable bases are, in general, inorganic compounds, such as alkali metal and alkaline earth metal hydroxides, such as LiOH, NaOH, KOH and Ca(OH)₂; alkali metal and alkaline earth metal oxides, such as lithium oxide, sodium oxide, calcium oxide, and magnesium oxide; alkali metal and alkaline earth metal hydrides, such as lithium hydride, sodium hydride, potassium hydride and calcium hydride; alkali metal and alkaline earth metal carbonates, such as lithium carbonate, potassium carbonate and calcium carbonate; and also alkali metal bicarbonates, such as sodium bicarbonate; moreover organic bases, for example tertiary amines, such as trimethylamine, triethylamine, triisopropylethylamine and N-methylpiperidine, pyridine; substituted pyridines, such as collidine, lutidine and 4-dimethylaminopyridine; and also bicyclic amines. Particular preference is given to alkali metal and alkaline earth metal hydroxides, such as LiOH, NaOH and KOH, preferably NaOH. The bases are generally employed in catalytic amounts; however, they can also be used in equimolar amounts, in excess or, if appropriate, as solvent. It is also possible to use mixtures of the bases mentioned.

Suitable catalysts are phase transfer catalysts, such as quaternary amines, for example tetra-n-butyl ammonium chloride, benzalkonium chloride, cetyl alkonium chloride, cetyl pyridinium chloride; and crown ethers, for example 12-crown-4, 15-crown-5, 18-crown-6, dibenzo-18-crown-6, and diaza-18-crown-6. Particular preference is given to quaternary amines, preferably tetra-n-butyl ammonium chloride.

The starting materials are generally reacted with one another in equimolar amounts. In terms of yield, it may be advantageous to employ an excess of hydroxylamine, based on XIX.

Alternatively, aldehyde compounds Via may be reacted with hydroxylamine directly, followed by reaction with a halogenating agent, and a 1,3-bipolar addition reaction with compounds XIII-B to isoxazoline compounds XIV-A, as described in Example S.6 of WO2010/125130.

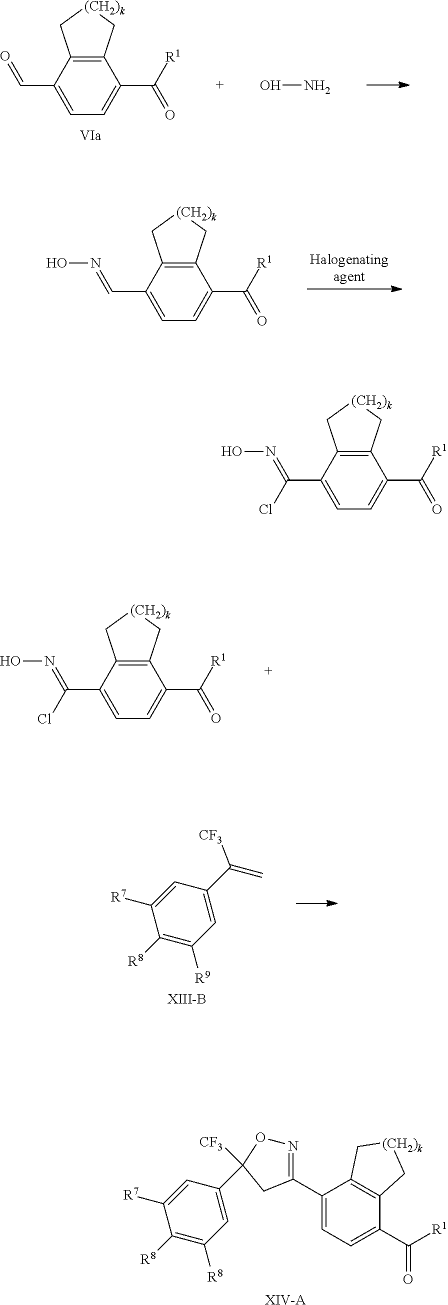

wherein all variables have a meaning as defined for compounds XIV-A.

Preparation of the oxime from compounds Via) is usually carried out at temperatures of from 10 to 40° C., preferably from 20 to 30° C., in an inert solvent in the presence of hydroxylamine, as described in Galvis et al., Org. Biomol. Chem., 2013, (11) 407-411, Supplementary Information.

Suitable solvents are aliphatic hydrocarbons such as pentane, hexane, cyclohexane, and petrol ether; aromatic hydrocarbons such as toluene, o-, m-, and p-xylene; halogenated hydrocarbons such as methylene chloride, dichloroethane, chloroform, and chlorobenzene; ethers such as diethylether, diisopropylether, tert-butylmethylether, dioxane, anisole, and tetrahydrofurane; nitriles such as acetonitrile, and propionitrile; alcohols such as CH₃OH, CH₃CH₂OH, CH₃CH₂CH₂OH, CH₃CH(OH)CH₃, CH₃(CH₂)₃OH, and C(CH₃)₃OH; moreover water DMSO, DMF, and DMA; preferably alcohols and water, in particular ethanol and water. It is also possible to use mixtures of the solvents mentioned.

Halogenation of the oxime to the hydroxamic acid is usually carried out at temperatures of from 50 to 90° C., preferably from 60 to 80° C., in an inert solvent in the presence of a halogenating agent.

Typical halogenating agents are oxalylchloride, thionylchloride, phorphortri- and pentachloride, phosphortri- and pentabromide, N-chlor- and N-bromosuccinimide, preferably thionylchloride, or N-chlor succinimmide.

Suitable solvents are aliphatic hydrocarbons such as pentane, hexane, cyclohexane, and petrol ether; aromatic hydrocarbons such as toluene, o-, m-, and p-xylene; halogenated hydrocarbons such as methylene chloride, dichloroethane, chloroform, and chlorobenzene; ethers such as diethylether, diisopropylether, tert-butylmethylether, dioxane, anisole, and tetrahydrofurane; nitriles such as acetonitrile, and propionitrile; ketones such as acetone, methyl ethyl ketone, diethyl ketone, and tert-butyl methyl ketone; moreover DMSO, DMF, and DMA; preferably DMF. It is also possible to use mixtures of the solvents mentioned.

Reaction of the hydroxamic acid halogenide with compounds XIII-B is usually carried out at temperatures of from −10 to 20° C., preferably from −5 to 5° C., in an inert solvent in the presence of a base.

Suitable solvents are aliphatic hydrocarbons such as pentane, hexane, cyclohexane, and petrol ether; aromatic hydrocarbons such as toluene, o-, m-, and p-xylene; halogenated hydrocarbons such as methylene chloride, dichloroethane, chloroform, and chlorobenzene; ethers such as diethylether, diisopropylether, tert-butylmethylether, dioxane, anisole, and tetrahydrofurane; nitriles such as acetonitrile, and propionitrile; ketones such as acetone, methyl ethyl ketone, diethyl ketone, and tert-butyl methyl ketone; moreover DMF, and DMA; preferably DMF. It is also possible to use mixtures of the solvents mentioned.

Suitable bases are, in general, inorganic compounds, such as alkali metal and alkaline earth metal hydroxides, such as LiOH, NaOH, KOH and Ca(OH)₂; alkali metal and alkaline earth metal oxides, such as lithium oxide, sodium oxide, calcium oxide, and magnesium oxide; alkali metal and alkaline earth metal hydrides, such as lithium hydride, sodium hydride, potassium hydride and calcium hydride; alkali metal and alkaline earth metal carbonates, such as lithium carbonate, potassium carbonate and calcium carbonate; and also alkali metal bicarbonates, such as sodium bicarbonate; moreover organic bases, for example tertiary amines, such as trimethylamine, triethylamine, triisopropylethylamine and N-methylpiperidine, pyridine; substituted pyridines, such as collidine, lutidine and 4-dimethylaminopyridine; and also bicyclic amines. Particular preference is given to organic bases, in particular tertiary amines. The bases are generally employed in catalytic amounts; however, they can also be used in equimolar amounts, in excess or, if appropriate, as solvent. It is also possible to use mixtures of the bases mentioned.

Isothiazoline compounds XIV-A, wherein V is N, and W is S, may be manufactured by the methods disclosed in WO2014/206911, Synthesis Example S.1.

Pyrroline compounds XIV-A, wherein V is N, and W is CH₂, may be manufactured by reacting compounds XIX with nitromethane, followed by reduction with a suitable reducing agent.

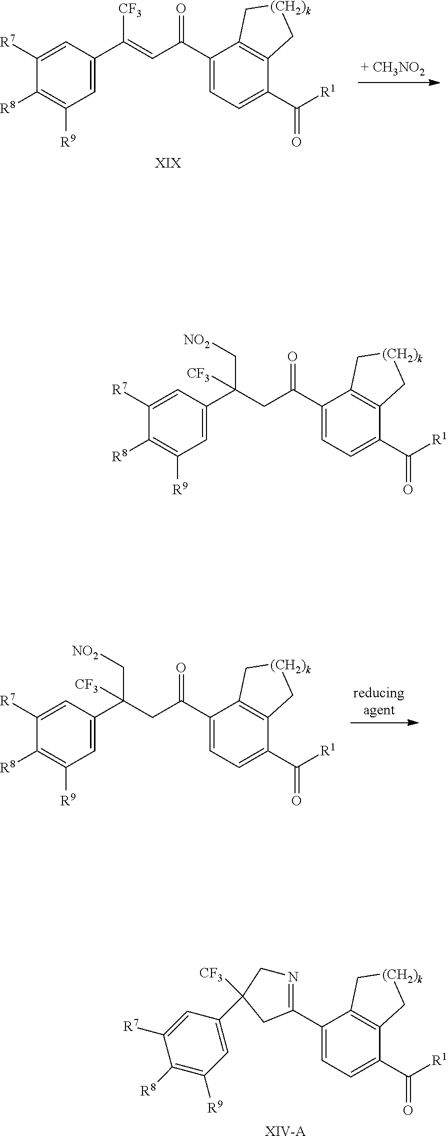

wherein all variables have a meaning as defined for compounds XIV-A.

The reaction of compounds XIX with nitromethane is usually carried out at temperatures of from −10 to 20° C., preferably from −5 to 5° C., in an inert solvent in the presence of a base.

Suitable solvents are aliphatic hydrocarbons such as pentane, hexane, cyclohexane, and petrol ether; aromatic hydrocarbons such as toluene, o-, m-, and p-xylene; halogenated hydrocarbons such as methylene chloride, dichloroethane, chloroform, and chlorobenzene; ethers such as diethylether, diisopropylether, tert-butylmethylether, dioxane, anisole, and tetrahydrofurane; nitriles such as acetonitrile, and propionitrile; ketones such as acetone, methyl ethyl ketone, diethyl ketone, and tert-butyl methyl ketone; alcohols such as CH₃OH, CH₃CH₂OH, CH₃CH₂CH₂OH, CH₃CH(OH)CH₃, CH₃(CH₂)₃OH, and C(CH₃)₃OH; moreover DMF, and DMA; preferably nitriles, and in particular acetonitrile. It is also possible to use mixtures of the solvents mentioned.

Suitable bases are, in general, inorganic compounds, such as alkali metal and alkaline earth metal hydroxides, such as LiOH, NaOH, KOH and Ca(OH)₂; alkali metal and alkaline earth metal oxides, such as lithium oxide, sodium oxide, calcium oxide, and magnesium oxide; alkali metal and alkaline earth metal hydrides, such as lithium hydride, sodium hydride, potassium hydride and calcium hydride; alkali metal and alkaline earth metal carbonates, such as lithium carbonate, potassium carbonate and calcium carbonate; and also alkali metal bicarbonates, such as sodium bicarbonate; moreover organic bases, for example tertiary amines, such as trimethylamine, triethylamine, triisopropylethylamine and N-methylpiperidine, pyridine; substituted pyridines, such as collidine, lutidine and 4-dimethylaminopyridine; and also polycyclic amines, such as 1,4-diazabicyclo[2.2.2]octane, or 1,8-diazabicyclo[5.4.0]un-dec-7-ene. Particular preference is given to organic bases, in particular polycyclic amines, such as 1,8-diazabicyclo[5.4.0]un-dec-7-ene. The bases are generally employed in catalytic amounts; however, they can also be used in equimolar amounts, in excess or, if appropriate, as solvent. It is also possible to use mixtures of the bases mentioned.

Reduction of the nitromethylated product to compounds XIV-A is usually carried out at temperatures of from 60 to 100° C., preferably from 70 to 90° C., in an inert solvent in the presence of an acid, and a reducing agent.

Suitable solvents are aliphatic hydrocarbons such as pentane, hexane, cyclohexane, and petrol ether; aromatic hydrocarbons such as toluene, o-, m-, and p-xylene; halogenated hydrocarbons such as methylene chloride, dichloroethane, chloroform, and chlorobenzene; ethers such as diethylether, diisopropylether, tert-butylmethylether, dioxane, anisole, and tetrahydrofurane; nitriles such as acetonitrile, and propionitrile; ketones such as acetone, methyl ethyl ketone, diethyl ketone, and tert-butyl methyl ketone; alcohols such as CH₃OH, CH₃CH₂OH, CH₃CH₂CH₂OH, CH₃CH(OH)CH₃, CH₃(CH₂)₃OH, and C(CH₃)₃OH; moreover water, DMSO, DMF, and DMA; preferably alcohols, and in particular CH₃OH. It is also possible to use mixtures of the solvents mentioned.

Suitable reducing agents are metals, for example alkaline metals, earth alkaline metals, and transition metals of period 4, metal salts metal oxides, such as salts or oxides of copper, tin, and lead; inorganic hydrides, such as NaH, NaBH₄, and LiAlH₄, alcohols, such as CH₃OH, CH₃CH₂OH, and CH₃CH(OH)CH₃; phosphines, such as triphenylphosphine, and trimethylphosphine; and other such as sulfite, dithionite, thiosulfate, hydrazine, aldehydes, preferably metals and inorganic hydrides, more preferably transition metals, such as Fe, Al, or Zn, in particular Fe.

Suitable acids are in general inorganic acids, such as hydrofluoric acid, hydrochloric acid, hydrobromic acid, sulphuric acid und perchloric acid; moreover organic acids, such as formic acid, acetic acid, propionic acid, oxalic acid, toluene sulphonic acid, benzene sulphonic acid, camphor sulphonic acid, citric acid, and trifluoro acetic acid; preferably organic acids, and in particular acetic acid. The acids are generally employed in catalytic amounts; however, they can also be used in equimolar amounts, in excess or, if appropriate, as solvent.

Dihydrothiphene compounds XIV-A, wherein V is CH, and W is S, may be manufactured by reacting compounds XIX with 2-sulfanylacetic acid, followed by an elimination reaction.

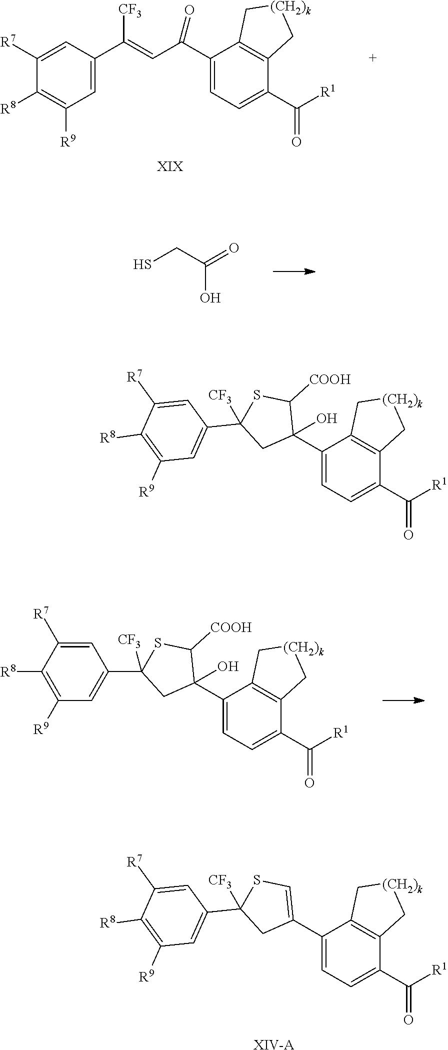

wherein all variables have a meaning as defined for compounds XIV-A.

Reaction of compounds XIX with 2-sulfanylacetic acid is usually carried out at temperatures of 10 to 40° C., preferably from 15 to 30° C. in an inert solvent in the presence of a base.

Suitable solvents are aliphatic hydrocarbons such as pentane, hexane, cyclohexane, and petrol ether; aromatic hydrocarbons such as toluene, o-, m-, and p-xylene; halogenated hydrocarbons such as methylene chloride, dichloroethane, chloroform, and chlorobenzene; ethers such as diethylether, diisopropylether, tert-butylmethylether, dioxane, anisole, and tetrahydrofurane; nitriles such as acetonitrile, and propionitrile; ketones such as acetone, methyl ethyl ketone, diethyl ketone, and tert-butyl methyl ketone; alcohols such as CH₃OH, CH₃CH₂OH, CH₃CH₂CH₂OH, CH₃CH(OH)CH₃, CH₃(CH₂)₃OH, and C(CH₃)₃OH; moreover DMSO, DMF, and DMA; preferably ethers, and in particular THF. It is also possible to use mixtures of the solvents mentioned.

Suitable bases are, in general, inorganic compounds, such as alkali metal and alkaline earth metal hydroxides, such as LiOH, NaOH, KOH and Ca(OH)₂; alkali metal and alkaline earth metal oxides, such as lithium oxide, sodium oxide, calcium oxide, and magnesium oxide; alkali metal and alkaline earth metal hydrides, such as lithium hydride, sodium hydride, potassium hydride and calcium hydride; alkali metal and alkaline earth metal carbonates, such as lithium carbonate, potassium carbonate and calcium carbonate; and also alkali metal bicarbonates, such as sodium bicarbonate; moreover organic bases, for example tertiary amines, such as trimethylamine, triethylamine, triisopropylethylamine and N-methylpiperidine, pyridine; substituted pyridines, such as collidine, lutidine and 4-dimethylaminopyridine; and also polycyclic amines, such as 1,4-diazabicyclo[2.2.2]octane, or 1,8-diazabicyclo[5.4.0]un-dec-7-ene. Particular preference is given to organic bases, in particular tertiary amines, such as trimethylamine. The bases are generally employed in catalytic amounts; however, they can also be used in equimolar amounts, in excess or, if appropriate, as solvent. It is also possible to use mixtures of the bases mentioned.

The subsequent elimination reaction to dihydrothiophene compounds XIV-A is usually carried out in two steps. Step one may be carried out at temperatures of −20 to 40° C., preferably from −10 to 10° C. in an inert solvent in the presence of a base and an acid halgenide.

Suitable acid halogenides are halogenides of organic acids with a pKa below 5, preferably below 2, such as mesyl chloride, tosyl chloride.

Suitable solvents in step one are aliphatic hydrocarbons such as pentane, hexane, cyclohexane, and petrol ether; aromatic hydrocarbons such as toluene, o-, m-, and p-xylene; halogenated hydrocarbons such as methylene chloride, dichloroethane, chloroform, and chlorobenzene; ethers such as diethylether, diisopropylether, tert-butylmethylether, dioxane, anisole, and tetrahydrofurane; nitriles such as acetonitrile, and propionitrile; ketones such as acetone, methyl ethyl ketone, diethyl ketone, and tert-butyl methyl ketone; alcohols such as CH₃OH, CH₃CH₂OH, CH₃CH₂CH₂OH, CH₃CH(OH)CH₃, CH₃(CH₂)₃OH, and C(CH₃)₃OH; moreover DMSO, DMF, and DMA. In one embodiment, no solvent is used apart from the base.

Suitable bases are, in general, inorganic compounds, such as alkali metal and alkaline earth metal hydroxides, such as LiOH, NaOH, KOH and Ca(OH)₂; alkali metal and alkaline earth metal oxides, such as lithium oxide, sodium oxide, calcium oxide, and magnesium oxide; alkali metal and alkaline earth metal hydrides, such as lithium hydride, sodium hydride, potassium hydride and calcium hydride; alkali metal and alkaline earth metal carbonates, such as lithium carbonate, potassium carbonate and calcium carbonate; and also alkali metal bicarbonates, such as sodium bicarbonate; moreover organic bases, for example tertiary amines, such as trimethylamine, triethylamine, triisopropylethylamine and N-methylpiperidine, pyridine; substituted pyridines, such as collidine, lutidine and 4-dimethylaminopyridine; and also polycyclic amines, such as 1,4-diazabicyclo[2.2.2]octane, or 1,8-diazabicyclo[5.4.0]un-dec-7-ene. Particular preference is given to organic bases, in particular tertiary amines, such as pyridine. The bases are generally employed in catalytic amounts; however, they can also be used in equimolar amounts, in excess or, if appropriate, as solvent. It is also possible to use mixtures of the bases mentioned.

Step two may be carried out at temperatures of 100 to 150° C., preferably from 110 to 130° C. in an inert solvent.

Suitable solvents in step two are aliphatic hydrocarbons such as pentane, hexane, cyclohexane, and petrol ether; aromatic hydrocarbons such as toluene, o-, m-, and p-xylene; halogenated hydrocarbons such as methylene chloride, dichloroethane, chloroform, and chlorobenzene; ethers such as diethylether, diisopropylether, tert-butylmethylether, dioxane, anisole, and tetrahydrofurane; nitriles such as acetonitrile, and propionitrile; ketones such as acetone, methyl ethyl ketone, diethyl ketone, and tert-butyl methyl ketone; alcohols such as CH₃OH, CH₃CH₂OH, CH₃CH₂CH₂OH, CH₃CH(OH)CH₃, CH₃(CH₂)₃OH, and C(CH₃)₃OH; moreover DMSO, DMF, and DMA; in particular DMF. It is also possible to use mixtures of the solvents mentioned.

Dihydrofurane compounds XIV-A may be produced by reaction of compounds XXI with compounds XXII

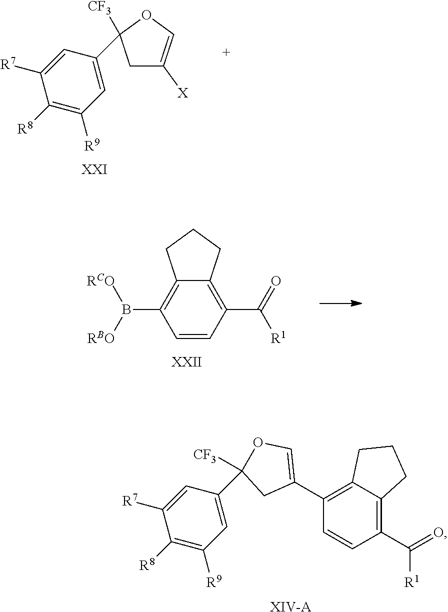

wherein X, R¹, and R^{C} have a meaning as defined for compounds I, and wherein all other variables have a meaning as defined for compounds XIV-A.

The reaction of compounds XXI and compounds XXII to compounds XIV-A is usually carried out in the presence of a transition metal, such as Ni, Zn, Pd, Pt, preferably Pd, and a ligand L in an inert solvent in the presence of a base. The transition metals are usually applied as salts, e.g. inorganic salts organic salts.

Suitable inert solvents for the reaction are aprotic polar and non-polar solvents. Typical solvents are aliphatic hydrocarbons, preferably C₅-C₁₆-alkanes, such as pentane, hexane, cyclohexane, and petrol ether; aromatic hydrocarbons, preferably C₆-C₁₀-aromatic hydrocarbons, such as toluene, o-, m-, and p-xylene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, dioxane, anisole, and THF, nitriles, preferably C₁-C₆-nitriles, such as CH₃CN, and propionitrile; ketones such as C₁-C₆-alkyl-C₁-C₆-alkyl ketones, C₁-C₆-alkyl-C₆-C₁-aryl ketones, and C₆-C₁₀-aryl-C₆-C₁₀-aryl-ketones, CH₃C(O)CH₃, CH₃C(O)CH₂CH₃, CH₃CH₂C(O)CH₂CH₃, and MTBK; DMSO, DMF, water, and DMA, preferably DMF, or water, most preferably a mixture of DMF and water, or DMF, and in particular DMF.

Typical Pd(II)-salts, which are converted to the catalytically active Pd(O) complex during the reaction, are PdO, PdCl₂, PdBr₂, PdI₂, Pd(NO₃)₂, PdSO₄, or Pd(OAc)₂, preferably PdCl₂ or Pd(OAc)₂, more preferably Pd(OAc)₂.

Suitable ligands L are phosphine derivatives, such as preferably mono-, bi- or tridentate phosphine derivatives, e.g. triphenylphosphine, tricyclohexylphosphine, phosphinooxazolines, 2,2′-bis(diphenylphosphino)-1,1′-binaphthyl), 2,2′-bis(diphenylphosphino)-1,1′-binaphthyl, bis(diphenylphosphino) methane, bis(diphenylphosphino) ethane, diphenyl-2-pyridylphosphine, and 1,3-bis(di-iso-propylphosphino)propane, preferably triphenylphosphine, diphenyl-2-pyridylphosphine, and 1,3-bis(di-iso-propylphosphino)propane. In one embodiment, the ligand L is bis(diphenylphosphino) ethane.

Suitable bases are inorganic bases and organic bases. Inorganic bases are usually alkali metal and alkaline earth metal hydroxides, such as LiOH, NaOH, KOH and Ca(OH)₂; alkali metal and alkaline earth metal oxides, such as lithium oxide, sodium oxide, calcium oxide, and magnesium oxide; alkali metal and alkaline earth metal hydrides, such as lithium hydride, sodium hydride, potassium hydride and calcium hydride; alkali metal and alkaline earth metal carbonates, such as lithium carbonate, potassium carbonate and calcium carbonate; alkali metal bicarbonates, such as sodium bicarbonate; and silver hydroxides or silver carbonates. Organic bases are usually tertiary amines, such as trimethylamine, triethylamine, 4-N,N-dimethylaminopyridine, triisopropylethylamine and N-methylpiperidine, pyridine, substituted pyridines, such as collidine, lutidine and 4-dimethylamino-pyridine, and also bicyclic amines; alkali metal acetates, such as NaOAc, KOAc; moreover secondary amines, such as, tetramethylethylendiamine, tetramethylene diamine, piperidine, diisopropylamine, morpholine, preferably alkali metal and alkaline earth metal hydroxides.

Compounds XXI are available by the methods described in WO2013/026726, or by derivatization of the compounds disclosed in therein. Compounds XXII may be produced from compounds I by processes described in WO2013/026726.

Typically, the reaction of compounds I to compounds XII is carried out in the presence of a boronic acid, a base, a transition metal such as Ni, Zn, Pd, Pt, preferably Pd, and a ligand L in an inert solvent. The transition metals are usually applied as salts, e.g. inorganic salts organic salts.

Suitable inert solvents are aliphatic hydrocarbons, preferably C₅-C₁₆-alkanes, such as pentane, hexane, cyclohexane, and petrol ether; aromatic hydrocarbons, preferably C₅-C₁₆-hydrocarbons, such as toluene, o-, m-, and p-xylene; ethers, such as CH₃CH₂OCH₂CH₃, (CH₃)₂CHOCH(CH₃)₂, MTBE, DME, dioxane, anisole, and THF; nitriles, preferably C₁-C₆-nitriles such as CH₃CN, and propionitrile; ketones such as C₁-C₆-alkyl-C₁-C₆-alkyl ketones, C₁-C₆-alkyl-C₆-C₁₀-aryl ketones, and C₆-C₁₀-aryl-C₆-C₁₀-aryl-ketones, CH₃C(O)CH₃, CH₃C(O)CH₂CH₃, CH₃CH₂C(O)CH₂CH₃, and MTBK; moreover dimethyl sulphoxide (DMSO), dimethyl formamide (DMF), and dimethylacetamide (DMA), preferably DMF.

Typical Pd(II)-salts, which are converted to the catalytically active Pd(0) complex during the reaction, are PdO, PdCl₂, PdBr₂, PdI₂, Pd(NO₃)₂, PdSO₄, or Pd(OAc)₂, preferably PdCl₂ or Pd(OAc)₂, more preferably PdCl₂.

Suitable ligands L are phosphine derivatives, such as preferably mono-, bi- or tridentate phosphine derivatives, e.g. triphenylphosphine, tricyclohexylphosphine, phosphinooxazolines, 2,2′-bis(diphenylphosphino)-1,1′-binaphthyl), 2,2′-bis(diphenylphosphino)-1,1′-binaphthyl, bis(diphenylphosphino) methane, bis(diphenylphosphino) ethane, diphenyl-2-pyridylphosphine, 1,1′-bis(diphenylphosphino)ferrocene, and 1,3-bis(di-iso-propylphosphino)propane, preferably 1,1′-bis(diphenylphosphino)ferrocene.

Suitable bases are inorganic bases and organic bases. Inorganic bases are usually alkali metal and alkaline earth metal hydroxides, such as LiOH, NaOH, KOH and Ca(OH)₂; alkali metal and alkaline earth metal oxides, such as lithium oxide, sodium oxide, calcium oxide, and magnesium oxide; alkali metal and alkaline earth metal hydrides, such as lithium hydride, sodium hydride, potassium hydride and calcium hydride; alkali metal and alkaline earth metal carbonates, such as lithium carbonate, sodium carbonate, potassium carbonate and calcium carbonate; alkali metal bicarbonates, such as sodium bicarbonate; and silver hydroxides or silver carbonates. Organic bases are usually tertiary amines, such as trimethylamine, triethylamine, 4-N,N-dimethylaminopyridine, triisopropylethylamine and N-methylpiperidine, pyridine, substituted pyridines, such as collidine, lutidine and 4-dimethylamino-pyridine, and also bicyclic amines; alkali metal acetates, such as NaOAc, KOAc; moreover secondary amines, such as, tetramethylethylendiamine, tetramethylene diamine, piperidine, diisopropylamine, morpholine, preferably alkali metal and alkaline earth metal carbonates, such as sodium carbonate.

Suitable boronic acids are derivatives of hypodiboric acid, such as bis(pinacolato)diboron, or tetramethyldiboron, preferably bis(pinacolato)diboron.

Compounds I, VI, and XIV are also intermediates for the manufacture of insecticidal compounds of formula XV

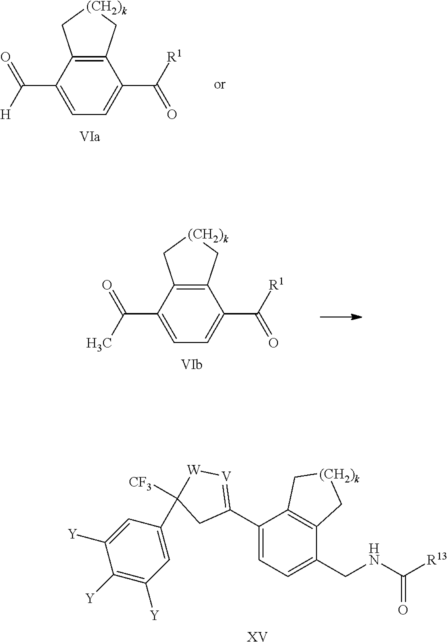

wherein each Y is independently a halogen, V is selected from CH and N, W is selected from O, S and CH₂, and R¹, R¹³ and k have a meaning as defined for compounds I. The meaning of the variables V, W, and Y, may have a meaning as defined for compounds XIV.

Compounds I, VI, and XIV-A are also intermediates for the manufacture of insecticidal compounds of formula XV-A

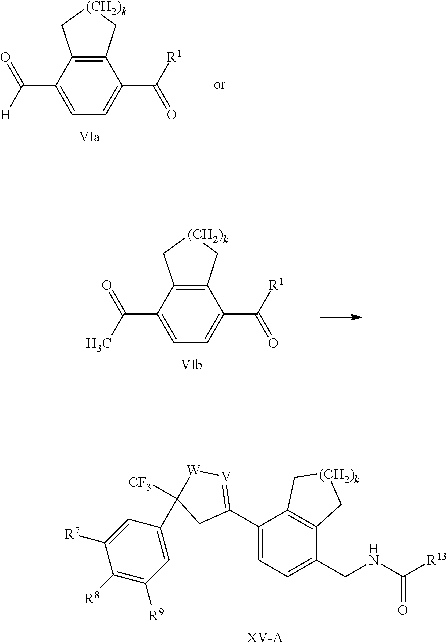

wherein V is selected from CH, N, and NO, W is selected from O, S and CH₂, and R¹, and R⁷, R⁸, and R⁹ are independently H, halogen, halomethyl, or halomethoxy, wherein at most two substituents R⁷, R⁸, and R⁹ are H, and R¹³ and k have a meaning as defined for compounds I. The meaning of the variables V, W, R⁷, R⁸, or R⁹, may have a meaning as defined for compounds XIV, or XIV-A.

Compounds I and VI, and XIV-B are also intermediates for the manufacture of insecticidal compounds XV-B

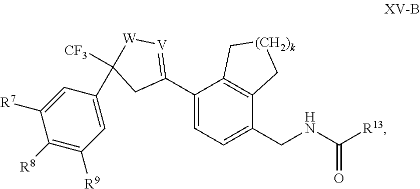

wherein V is selected from CH, N, and NO, W is selected from O, S and CH₂, R¹³ and k have a meaning as defined for compounds I, and R⁷, R⁸, and R⁹ are selected from a line A-1 to A-31 of Table 1. Compounds XV, and XV-B fall under the definition of compounds XV-A and are thus preferred embodiments of XV-A for all compounds and reactions disclosed herein.

In one embodiment, compounds XV-A are selected from

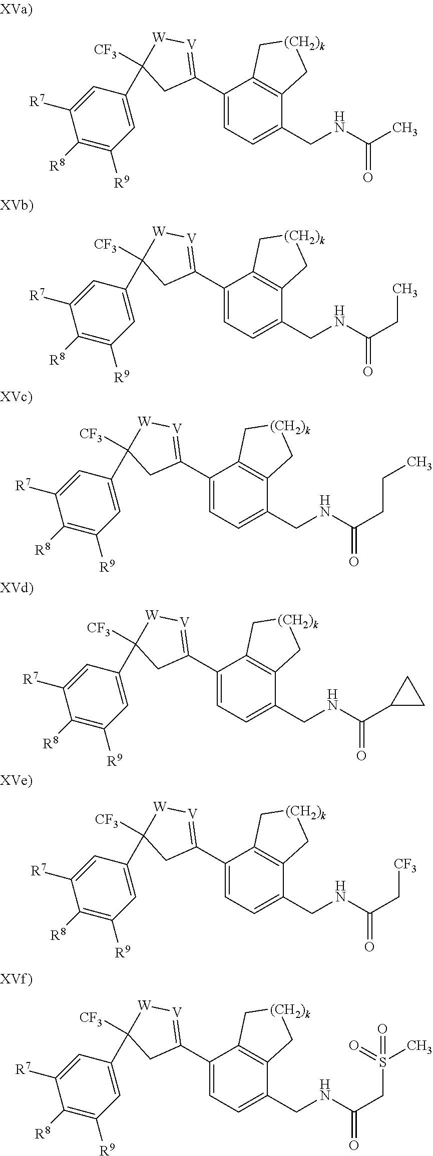

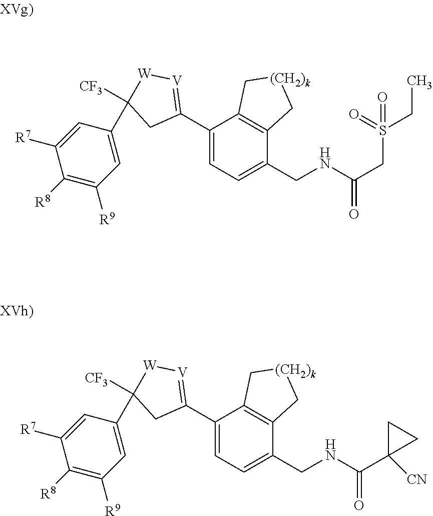

where all variables have a meaning as defined form compounds XV-A. In another embodiment, R⁷, R⁸, and R⁹ are independently halogen.

In another embodiment, compounds XV-A are selected from compounds XVa to XVg. In another embodiment, compounds XV-A are compounds XVh.

Suitable manufacture processes including suitable reaction conditions of insecticidal products XV-A by reaction of compounds VI, or compounds I are disclosed in U.S. 62/095,073 and U.S. 62/095,071, e.g. in case R¹ is H, reductive amination, or Leuckart-Wallach-reaction, followed by amidation:

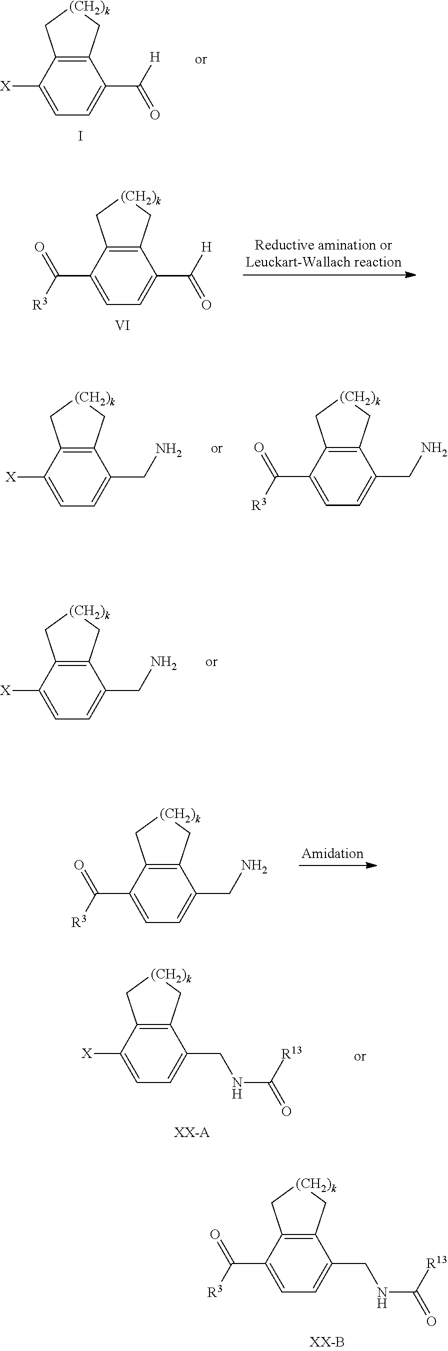

Reductive amination of compounds I, or compounds VI, is usually carried out in the presence of NH₃, or an ammonium salt, and a reducing agent, in an inert solvent.

Suitable solvents are aliphatic hydrocarbons, preferably C₅-C₁₆-alkanes, such as pentane, hexane, cyclohexane, and petrol ether; aromatic hydrocarbons, preferably C₅-C₁₆-hydrocarbons, such as toluene, o-, m-, and p-xylene; halogenated hydrocarbons, preferably halogenated C₁-C₆-alkanes or halogenated C₆-C₁₀ aromatic hydrocarbons, such as CH₂Cl₂, CHCl₃, and chlorobenzene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, dioxane, anisole, and THF; and alcohols such as methanol, ethanol, n-propanol, isopropanol, n-butanol, and tert-butanol, moreover DMSO, DMF, and DMA.

Suitable ammonium salts are ammonium halogenides, such as NH₄F, NH₄Cl, NH₄Br, or NH₄I, preferably NH₄Cl.

Suitable reducing agents are H₂, inorganic hydrides, such as NaH, NaBH₄, and LiAlH₄, BH₃, or its salts, such as NaBH₃CN, and formic acid. In case H₂ is used, it may be used in combination with a catalyst, such as Pd on active coal, or Raney Nickel, preferably Raney Nickel.

Amidation of the resulting amine to compounds XX-A, or compounds XX-B, is usually carried out by reaction with R¹³—COOH in the presence of an activating agent, or a coupling agent in an inert solvent. Suitable solvents are aliphatic hydrocarbons, preferably C₅-C₁₆-alkanes, such as pentane, hexane, cyclohexane, and petrol ether; aromatic hydrocarbons, preferably C₅-C₁₆-alkanes, such as toluene, o-, m-, and p-xylene; halogenated hydrocarbons, preferably halogenated C₁-C₆-alkanes and halogenated C₆-C₁₀-aromats, such as CH₂Cl₂, CHCl₃, and chlorobenzene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, dioxane, anisole, and THF; nitriles, preferably C₁-C₆-nitriles, such as CH₃CN, and propionitrile; ketones, preferably C₁-C₆-alkyl-C₁-C₆-alkyl ketones, such as CH₃C(O)CH₃, CH₃C(O)CH₂CH₃, CH₃CH₂C(O)CH₂CH₃, and MTBK; moreover DMSO, DMF, and DMA, preferably DMF. It is also possible to use mixtures of the solvents mentioned.

Suitable activating agents are halogenating agent, which are usually selected from chlorinating agents and brominating agents, such as oxalylchloride, thionylchloride, phosphortri- and pentabromide, phorphortri- and pentachloride, preferably from thionylchloride and oxalylchloride. Suitable coupling agents are well known and are for instance selected from carbodiimides, such as DCC (dicyclohexylcarbodiimide) and DIC (diisopropylcarbodiimide), benzotriazole derivatives, such as HATU (O-(7-azabenzotriazol-1-yl)-N,N,N′,N′-tetramethyluronium hexafluorophosphate), HBTU ((Obenzotriazol-1-yl)-N,N,N′,N′-tetramethyluronium hexafluorophosphate) and HCTU (1H-benzotriazolium-1-[bis(dimethylamino)methylene]-5-chloro tetrafluoroborate) and phosphonium-derived activators, such as BOP, PyBOP, and PyBrOP, preferably PyBrOP. Generally, the activating agent, or the coupling agent is used in excess.

Alternatively, amidation of the resulting amine to compounds XX-A, or compounds XX-B, may also be carried out by reaction with an ester derivative of R¹³—COOH at temperatures from 20 to 80° C., preferably from 30 to 70° C., more preferably from 40 to 60° C., and in particular from 45 to 55° C., in the presence of a catalyst, such as a metalorganic compound. Such reactions have been described by Levin et al., Synthetic Communications, 1982, (12) 989-993.

Suitable solvents are aliphatic hydrocarbons, preferably C₅-C₁₆-alkanes, such as pentane, hexane, cyclohexane, and petrol ether; aromatic hydrocarbons, preferably C₅-C₁₆-alkanes, such as toluene, o-, m-, and p-xylene, halogenated hydrocarbons, preferably halogenated C—C₆-alkanes and halogenated C₆-C₁₀-aromats, such as CH₂Cl₂, CHCl₃, and chlorobenzene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, dioxane, anisole, and THF; nitriles, preferably C₁-C₆-nitriles, such as CH₃CN, and propionitrile; ketones, preferably C₁-C₆-alkyl-C₁-C₆-alkyl ketones, such as CH₃C(O)CH₃, CH₃C(O)CH₂CH₃, CH₃CH₂C(O)CH₂CH₃, and MTBK; alcohols, preferably C₁-C₄-alcohols, such as CH₃OH, CH₃CH₂OH, CH₃CH₂CH₂OH, CH₃CH(OH)CH₃, CH₃(CH₂)₃OH, and C(CH₃)₃OH; moreover DMSO, DMF, and DMA. Preferred solvents are CH₃C(O)CH₃, CH₃CN, CH₃NO₂, CHCl₃, CH₂Cl₂, CCl₄, CH₂ClCH₂Cl, benzene, toluene, xylene CH₃CH₂OCH₂CH₃, CH₃OCH₃, petroleum ether, C₅-C₁₂-alkanes, preferably CH₂Cl₂ and benzene, more preferably benzene. It is also possible to use mixtures of the solvents mentioned.

Suitable metalorganic compounds are C₁-C₆-alkyl metal or C₆-C₁₀-aryl metal compounds, preferably of Fe, Ti, Zr, Al, more preferably Al, such as Al(CH₃)₃, Al(CH₂CH₃)₂, Al(CH₂CH₂CH₃)₃, Al(CH(CH₃)₂)₃, Al(CH₂CH₂CH₂CH₃)₃, Al(CH(CH₃)(CH₂CH₃)₃, tri-tert-butyl aluminium, or Al(C₆H₅)₃, preferably Al(CH₃)₃.

Compounds XX-A may then be converted to compounds XIV-A, as described for the reaction of compounds I to compounds VI. Compounds XX-B may be converted to compounds XV-A, as described for the conversion of compounds VI to compounds XV-A.

Compounds XV-A may also be produced by reduction of the ester or carboxylic acid group of compounds XIV-A with R¹ being OR¹¹, followed by substitution of the resulting hydroxyl with an amine, and finally an amidation reaction:

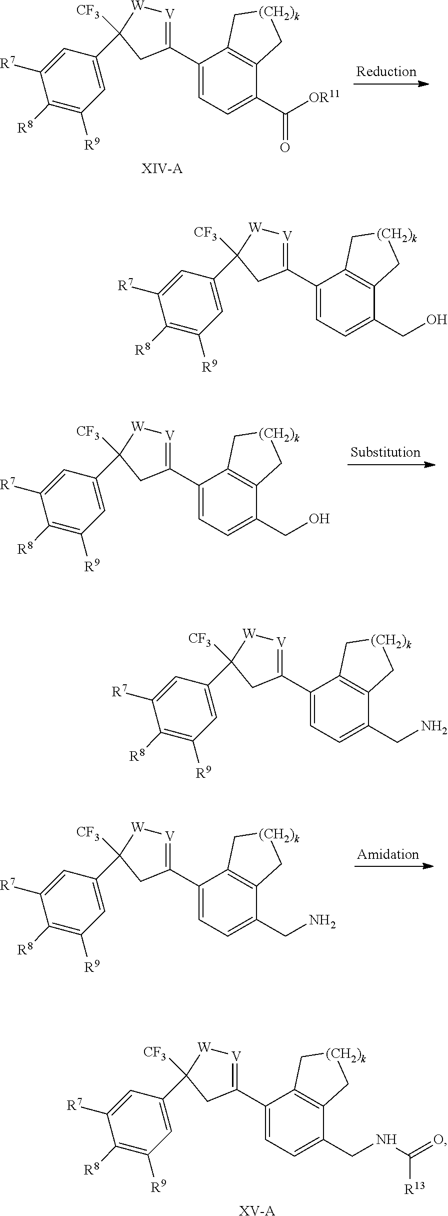

wherein all variables have a meaning as defined for compounds XV-A.

Reduction of the ester, or carboxylic acid group in compounds XIV-A is usually carried out at temperatures of from 10 to 30° C., in an inert solvent, in the presence of a reducing agent.

Suitable solvents are aliphatic hydrocarbons such as pentane, hexane, cyclohexane, and petrol ether; aromatic hydrocarbons such as toluene, o-, m-, and p-xylene; halogenated hydrocarbons such as methylene chloride, dichloroethane, chloroform, and chlorobenzene; ethers such as diethylether, diisopropylether, tert-butylmethylether, dioxane, anisole, and tetrahydrofurane; nitriles such as acetonitrile, and propionitrile; ketones such as acetone, methyl ethyl ketone, diethyl ketone, and tert-butyl methyl ketone; alcohols such as CH₃OH, CH₃CH₂OH, CH₃CH₂CH₂OH, CH₃CH(OH)CH₃, CH₃(CH₂)₃OH, and C(CH₃)₃OH; moreover DMSO, DMF, and DMA; in particular ethers, such as THF. It is also possible to use mixtures of the solvents mentioned.

Suitable reducing agents are metals, for example alkaline, and earth alkaline metals, metal salts metal oxides, such as salts or oxides of copper, tin, and lead; inorganic hydrides, such as NaH, NaBH₄, and LiAlH₄, alcohols, such as CH₃OH, CH₃CH₂OH, and CH₃CH(OH)CH₃; and other such as sulfite, dithionite, thiosulfate, hydrazine, aldehydes, preferably inorganic hydrides, and in particular LiBH₄.

Substitution of the hydroxyl with an amine is usually a process consisting of the activation of the hydroxyl group in step one, followed by nucleophilic substitution in step two.

Activation of the hydroxyl group is usually carried out at temperatures of from −10 to 50° C., in an inert solvent, in the presence of an acid halogenide and a base.

Suitable acid halogenides are halogenides of organic acids with a pKa below 5, preferably below 2, such as mesyl chloride, or tosyl chloride.

Suitable solvents are aliphatic hydrocarbons such as pentane, hexane, cyclohexane, and petrol ether; aromatic hydrocarbons such as toluene, o-, m-, and p-xylene; halogenated hydrocarbons such as methylene chloride, dichloroethane, chloroform, and chlorobenzene; ethers such as diethylether, diisopropylether, tert-butylmethylether, dioxane, anisole, and tetrahydrofurane; nitriles such as acetonitrile, and propionitrile; ketones such as acetone, methyl ethyl ketone, diethyl ketone, and tert-butyl methyl ketone; alcohols such as CH₃OH, CH₃CH₂OH, CH₃CH₂CH₂OH, CH₃CH(OH)CH₃, CH₃(CH₂)₃OH, and C(CH₃)₃OH; moreover DMSO, DMF, and DMA; in particular halogenated hydrocarbons, for example methylene chloride.

Suitable bases are, in general, inorganic bases, such as alkali metal and alkaline earth metal hydroxides, such as LiOH, NaOH, KOH and Ca(OH)₂, alkali metal and alkaline earth metal oxides, such as lithium oxide, sodium oxide, calcium oxide, and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate; moreover organic bases, for example tertiary amines, such as trimethylamine, triethylamine, triisopropylethylamine and N-methylpiperidine, pyridine, substituted pyridines, such as collidine, lutidine and 4-dimethylaminopyridine, and also bicyclic amines; in particular tertiary amines, such as triethylamine.

Nucleophilic substitution is then is usually carried out at temperatures of from 10 to 50° C., preferably from 20 to 30° C., in an inert solvent, in the presence of a nitrogen source.

Suitable solvents are aliphatic hydrocarbons such as pentane, hexane, cyclohexane, and petrol ether; aromatic hydrocarbons such as toluene, o-, m-, and p-xylene; halogenated hydrocarbons such as methylene chloride, dichloroethane, chloroform, and chlorobenzene; ethers such as diethylether, diisopropylether, tert-butylmethylether, dioxane, anisole, and tetrahydrofurane; nitriles such as acetonitrile, and propionitrile; ketones such as acetone, methyl ethyl ketone, diethyl ketone, and tert-butyl methyl ketone; alcohols such as CH₃OH, CH₃CH₂OH, CH₃CH₂CH₂OH, CH₃CH(OH)CH₃, CH₃(CH₂)₃OH, and C(CH₃)₃OH; moreover DMSO, DMF, and DMA; in particular DMF.

Suitable nitrogen sources are NH₃, primary- and secondary amines, and azides, preferably azide salts, and in particular NaN₃.

In case an azide is used in the nucleophilic substitution, reduction of the resulting azide product by a reducing agent in an inert solvent at temperatures of from 60 to 120° C., preferably 70 to 90° C.

Suitable solvents are aliphatic hydrocarbons such as pentane, hexane, cyclohexane, and petrol ether; aromatic hydrocarbons such as toluene, o-, m-, and p-xylene; halogenated hydrocarbons such as methylene chloride, dichloroethane, chloroform, and chlorobenzene; ethers such as diethylether, diisopropylether, tert-butylmethylether, dioxane, anisole, and tetrahydrofurane; nitriles such as acetonitrile, and propionitrile; ketones such as acetone, methyl ethyl ketone, diethyl ketone, and tert-butyl methyl ketone; alcohols such as CH₃OH, CH₃CH₂OH, CH₃CH₂CH₂OH, CH₃CH(OH)CH₃, CH₃(CH₂)₃OH, and C(CH₃)₃OH; moreover water, DMSO, DMF, and DMA; in particular water and THF.

Suitable reducing agents are metals, for example alkaline, and earth alkaline metals, metal salts metal oxides, such as salts or oxides of copper, tin, and lead; inorganic hydrides, such as NaH, NaBH₄, and LiAlH₄, alcohols, such as CH₃OH, CH₃CH₂OH, and CH₃CH(OH)CH₃; phosphines, such as triphenylphosphine, and trimethylphosphin; and other such as sulfite, dithionite, thiosulfate, hydrazine, aldehydes, preferably phosphines, and in particular triphenylphosphine.

Amidation reaction of the resulting amines to compounds XV-A is usually carried out at temperatures of from 10 to 50° C., preferably from 20 to 30° C., in an inert solvent, in the presence of a base, and an carboxylic acid halogenide derivative of R¹³—COOH, or a carboxylic acid R¹³—COOH and a coupling agent.

Suitable solvents are aliphatic hydrocarbons such as pentane, hexane, cyclohexane, and petrol ether; aromatic hydrocarbons such as toluene, o-, m-, and p-xylene; halogenated hydrocarbons such as methylene chloride, dichloroethane, chloroform, and chlorobenzene; ethers such as diethylether, diisopropylether, tert-butylmethylether, dioxane, anisole, and tetrahydrofurane; nitriles such as acetonitrile, and propionitrile; ketones such as acetone, methyl ethyl ketone, diethyl ketone, and tert-butyl methyl ketone; alcohols such as CH₃OH, CH₃CH₂OH, CH₃CH₂CH₂OH, CH₃CH(OH)CH₃, CH₃(CH₂)₃OH, and C(CH₃)₃OH; moreover water, DMSO, DMF, and DMA; ethers and DMF, and in particular ethers, such as THF.

Suitable coupling agents selected from carbodiimides, such as DCC (dicyclohexylcarbodiimide) and DIC (diisopropylcarbodiimide), benzotriazole derivatives, such as HATU (O-(7-azabenzotriazol-1-yl)-N,N,N′,N′-tetramethyluronium hexafluorophosphate), HBTU ((Obenzotriazol-1-yl)-N,N,N′,N′-tetramethyluronium hexafluorophosphate) and HCTU (1H-benzotriazolium-1-[bis(dimethylamino)methylene]-5-chloro tetrafluoroborate) and phosphonium-derived activators, such as BOP ((benzotriazol-1-yloxy)-tris(dimethylamino) phosphonium hexafluorophosphate), PyBOP ((benzotriazol-1-yloxy)-tripyrrolidinphosphonium hexafluorophosphate) and PyBrOP (bromotripyrrolidinphosphonium hexafluorophosphate), preferably PyBrOP. Generally, the coupling agent is used in excess.

Typical bases applied are organic bases, such as pyridine, 4-N,N-dimethylamino-pyridine, tetramethylene diamine, piperidine, diisopropylamine, morpholine, and triethylamine, preferably pyridine, 4-N,N-dimethylaminopyridine, and diisopropylamine, in particular tertiary amines, such as trimethylamine, diisopropylethylamine, triethylamine, triisopropylethylamine and N-methylpiperidine, pyridine, substituted pyridines, such as collidine, lutidine and 4-dimethylaminopyridine, especially triethylamine.

Compounds I are also suitable intermediates for the manufacture of insecticidal compounds of formula XVI

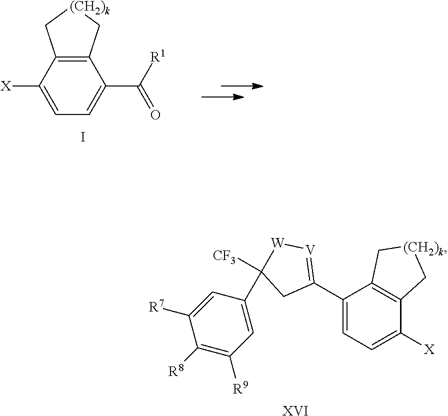

wherein X is halogen, and all other variables have a meaning as defined for compounds XIV-A.

This process usually involves the conversion of compounds I to compounds XVII

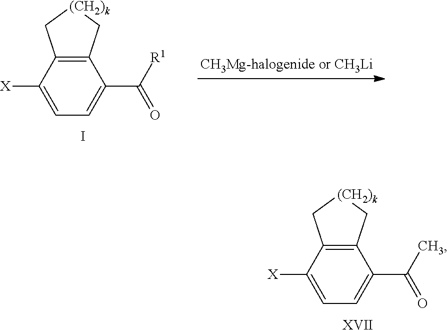

where all variables have a meaning as defined for compounds I.

This transformation is usually carried an aprotic polar or non-polar solvent in the presence of CH₃Mg-halogenide, or CH₃Li.

The reaction is preferably carried out at temperatures of from −78 to 110° C., preferably at temperatures from −50 to 20° C., more preferably from −20 to 0° C.

Suitable solvents for the reaction are aprotic polar and non-polar solvents. Typical solvents are aliphatic hydrocarbons, preferably C₅-C₁₆-alkanes, such as pentane, hexane, cyclohexane, and petrol ether; aromatic hydrocarbons, preferably C₅-C₁₆-alkanes, such as toluene, o-, m-, and p-xylene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, dioxane, anisole, and THF.

The reaction is preferably carried out in aromatic hydrocarbons, preferably C₅-C₁₆-alkanes, such as benzene, toluene, xylenes, cumene, chlorobenzene, nitrobenzene, or tert-butylbenzene, aprotic polar solvents, for example cyclic, or acyclic ethers, such as diethyl ether, tert-butyl methyl ether (MTBE), cyclopentyl methyl ether, THF or dioxane.

The CH₃Mg-halogenide may be CH₃MgF, CH₃MgCl, CH₃MgBr, or CH₃MgI, preferably CH₃MgBr.

The molar ratio of the CH₃Mg-halogenide, or CH₃Li to compounds I is usually from 1:1 to 5:1, preferably from 1:1 to 2:1, and most preferably from 1:1 to 1:1.5.

Compounds XVII may then be converted to compounds XVI by methods disclosed in WO 2010/125130, WO02015128358, WO02014206908, and WO02014206910. Compounds XVI may be converted to compounds XIV, or compounds XV-A, by the reactions listed above, which are also described in WO2015128358

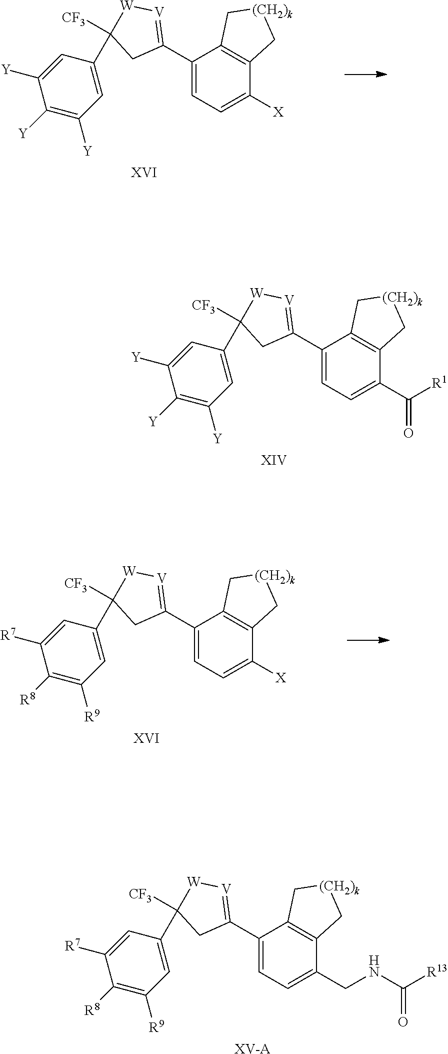

where X, R¹, and R¹³ have a meaning as defined for compounds I, Y is independently halogen, and wherein all other variables have a meaning as defined for compounds XV-A.

Another aspect of the invention is the use of compounds I or II for the manufacture of such insecticidal compounds XIV-A, or XV-A; and methods for the manufacture of insecticidal products XIV-A, or XV-A from compounds I or II.

Suitable processes for the production of compounds VI from compounds I or II have been described above. Further conversion of compounds VI to compounds XIV-A, or XV-A can be carried out by the methods described above.

Compounds VI, and insecticidal compounds XIV-A with R¹ being OR¹¹ and R¹¹ being not H can be hydrolyzed to the respective carboxylic acid.

This process is usually carried out in the presence of a base or an acid in an inert solvent, and optionally H₂O.

Suitable solvents are aliphatic hydrocarbons, preferably C₅-C₁₆-alkanes, such as pentane, hexane, cyclohexane, and petrol ether; aromatic hydrocarbons, preferably C₅-C₁₆-alkanes, such as toluene, o-, m-, and p-xylene; halogenated hydrocarbons, preferably halogenated C₁-C₆-alkanes and halogenated C₆-C₁₀-aromats, such as CH₂Cl₂, CHCl₃, and chlorobenzene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, dioxane, anisole, and THF; nitriles, preferably C₁-C₆-nitriles, such as CH₃CN, and propionitrile; ketones, preferably C₁-C₆-alkyl-C₁-C₆-alkyl ketones, such as CH₃C(O)CH₃, CH₃C(O)CH₂CH₃, CH₃CH₂C(O)CH₂CH₃, and MTBK; alcohols, preferably C₁-C₄-alcohols, such as CH₃OH, CH₃CH₂OH, CH₃CH₂CH₂OH, CH₃CH(OH)CH₃, CH₃(CH₂)₃OH, and C(CH₃)₃OH; moreover DMSO, DMF, and DMA. Preferred solvents are C₁-C₆-alkyl-C₁-C₆-alkyl ethers and C₁-C₆-alkyl-C₆-C₁₀-aryl ethers, in particular dioxane and THF. It is also possible to use mixtures of the solvents mentioned.

Suitable acids are mineral acids, such as hydrochloric acid, sulfuric acid organic acids, such as trifluoroacetic acid. Suitable bases are alkali metal hydroxides and earth alkali metal hydroxides, such as LiOH, NaOH or KOH.

Compounds VI, and insecticidal compounds XIV-A, or XV-A with R¹ being OH may be reacted with an amine NHR¹²R¹³ to the amide.

This process is usually carried out in an inert solvent, in the presence of a base and by activation with an activating agent, or a coupling agent.

Suitable solvents are aliphatic hydrocarbons, preferably C₅-C₁₆-alkanes, such as pentane, hexane, cyclohexane, and petrol ether; aromatic hydrocarbons, preferably C₅-C₁₆-alkanes, such as toluene, o-, m-, and p-xylene; halogenated hydrocarbons, preferably halogenated C₁-C₆-alkanes and halogenated C₆-C₁₀-aromatic hydrocarbons, such as CH₂Cl₂, CHCl₃, and chlorobenzene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, dioxane, anisole, and THF; nitriles, preferably C₁-C₆-nitriles, such as CH₃CN, and propionitrile; ketones, preferably C₁-C₆-alkyl-C₁-C₆-alkyl ketones, such as CH₃C(O)CH₃, CH₃C(O)CH₂CH₃, CH₃CH₂C(O)CH₂CH₃, and MTBK; moreover DMSO, DMF, and DMA, preferably DMF. It is also possible to use mixtures of the solvents mentioned.

Suitable activating agents are halogenating agent, which are usually selected from chlorinating agents and brominating agents, such as oxalylchloride, thionylchloride, phosphortri- and pentabromide, phorphortri- and pentachloride, preferably from thionylchloride and oxalylchloride. Suitable coupling agents are well known and are for instance selected from carbodiimides, such as DCC (dicyclohexylcarbodiimide) and DIC (diisopropylcarbodiimide), benzotriazole derivatives, such as HATU (O-(7-azabenzotriazol-1-yl)-N,N,N′,N′-tetramethyluronium hexafluorophosphate), HBTU ((Obenzotriazol-1-yl)-N,N,N′,N′-tetramethyluronium hexafluorophosphate) and HCTU (1H-benzotriazolium-1-[bis(dimethylamino)methylene]-5-chloro tetrafluoroborate) and phosphonium-derived activators, such as BOP, PyBOP, and PyBrOP, preferably PyBrOP. Generally, the activating agent is used in excess.

As a further alternative insecticidal compounds VI, and insecticidal compounds XIV-A, or XV-A with R¹ being OR¹¹ and R¹¹ being not H (ester form) can also be directly converted to the corresponding amide.

This process is usually carried out at temperatures from 20 to 80° C., preferably from 30 to 70° C., more preferably from 40 to 60° C., and in particular from 45 to 55° C., in the presence of a catalyst, such as a metalorganic compound. Such reactions have been described by Levin et al., Synthetic Communications, 1982, (12) 989-993.

Suitable solvents are aliphatic hydrocarbons, preferably C₅-C₁₆-alkanes, such as pentane, hexane, cyclohexane, and petrol ether; aromatic hydrocarbons, preferably C₅-C₁₆-alkanes, such as toluene, o-, m-, and p-xylene, halogenated hydrocarbons, preferably halogenated C—C₆-alkanes and halogenated C₆-C₁₀-aromats, such as CH₂Cl₂, CHCl₃, and chlorobenzene; ethers, preferably C₁-C₆-alkyl-C₁-C₆-alkyl ethers and C₁-C₆-alkyl-C₆-C₁₀-aryl ethers, such as CH₃CH₂OCH₂CH₃, (CH₃)₂CHOCH(CH₃)₂, MTBE, DME, dioxane, anisole, and THF; nitriles, preferably C₁-C₆-nitriles, such as CH₃CN, and propionitrile; ketones, preferably C₁-C₆-alkyl-C₁-C₆-alkyl ketones, such as CH₃C(O)CH₃, CH₃C(O)CH₂CH₃, CH₃CH₂C(O)CH₂CH₃, and MTBK; alcohols, preferably C₁-C₄-alcohols, such as CH₃OH, CH₃CH₂OH, CH₃CH₂CH₂OH, CH₃CH(OH)CH₃, CH₃(CH₂)₃OH, and C(CH₃)₃OH; moreover DMSO, DMF, and DMA. Preferred solvents are CH₃C(O)CH₃, CH₃CN, CH₃NO₂, CHCl₃, CH₂C₂, CCl₄, CH₂ClCH₂Cl, benzene, toluene, xylene CH₃CH₂OCH₂CH₃, CH₃OCH₃, petroleum ether, C₅-C₁₂-alkanes, preferably CH₂Cl₂ and benzene, more preferably benzene. It is also possible to use mixtures of the solvents mentioned.

Suitable metalorganic compounds are C₁-C₆-alkyl metal or C₆-C₁₀-aryl metal compounds, preferably of Fe, Ti, Zr, Al, more preferably Al, such as Al(CH₃)₃, Al(CH₂CH₃)₂, Al(CH₂CH₂CH₃)₃, Al(CH(CH₃)₂)₃, Al(CH₂CH₂CH₂CH₃)₃, Al(CH(CH₃)(CH₂CH₃)₃), Al(C(CH₃)₃)₃, or triphenylaluminium, preferably Al(CH₃)₃.

The initial starting compounds Xa and Xb are commercially available. Compounds of formula XII-A are commercially available, or may be produced by commercially available stannane chlorides with Grignard reagents. Compounds XII-B are commercially available, or can be produced from vinyl alcohol by a Williamson ether synthesis. Compounds XIII-A can be produced as described in CN103224447, or Raja et al., Tetrahedron Letters, 2011, 52(40), p. 5170-5172. Compounds XIII-A are also commercially available. Compounds XIII-B may be produced as described in WO2010/125130. In case any educts for the reactions disclosed herein are not commercially available, or directly obtainable by the methods described in the prior art given above, they can be produced by derivatization of these compounds.

The reaction mixtures are worked up in a customary manner, for example by mixing with H₂O, separating the phases and, if appropriate, chromatographic purification of the crude products. Some of the intermediates and end products are produced in the form of colorless or slightly brownish viscous oils which are purified or freed from volatile components under reduced pressure and at moderately elevated temperature. If the intermediates and end products are produced as solids, purification can also be carried out by recrystallization or digestion.

If individual compounds I, II, or VI cannot be produced by the routes described above, they can be produced by derivatization of other compounds I, II, or VI.

However, if the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant, or in the harmful fungus to be controlled.

The terms for organic groups used in the definition of the variables, such as, for example, the term “halogen”, are collective terms which represent the individual members of these groups of organic moieties. In each case, the prefix Cₓ-C_{y} denotes the number of possible carbon atoms.

The term “halogen” refers in each case to fluorine, chlorine, bromine or iodine, especially fluorine or chlorine. In another embodiment, the term halogen refers to chlorine, bromine, or iodine. In yet another embodiment, the term halogen refers to bromine, or iodine. In yet another embodiment, the term halogen refers to bromine.

In all above cases C₅-C₁₂ alkanes means n-pentane, n-hexane, n-heptane, n-octane, n-nonane, n-decane, n-undecane, n-dodocane, isopentane, neopentane, 2-methyl pentane, 3-methyl pentane, 2,2,-dimethylpentane, as well as all isomers of heptane, octane, nonane, decane, undecane, and dodecane, and the mixture of the aforementioned C₅-C₁₂ alkanes.

The term “alkyl”, as used in C₁-C₆-alkyl, C₁-C₄-alkyl and in the terms C₁-C₆-alkoxy, refers to a saturated straight-chain or branched hydrocarbon group, for example methyl, ethyl, propyl, 1-methylethyl, butyl, 1-methylpropyl, 2-methylpropyl, 1,1-dimethylethyl, pentyl, 1-methylbutyl, 2-ethylbutyl, 3-methylbutyl, 1,1-dimethylpropyl, 1,2-dimethylpropyl, 2,2-dimethylpropyl, 1-ethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl. The term “C₂-C₆-alkenyl” refers to monounsaturated straight-chain or branched hydrocarbon radicals having 2 to 6 carbon atoms, and a C—C double bond in any position, such as ethenyl, 1-propenyl, 2-propenyl, 1-methylethenyl, 1-butenyl, 2-butenyl, or 3-butenyl.

The term “C₂-C₆-alkynyl” refers to monounsaturated straight-chain or branched hydrocarbon radicals having 2 to 6 carbon atoms, and a C—C triple bond in any position, for example C₂-C₆-alkynyl, such as ethynyl, 1-propynyl, and 2-propynyl.

The term “C₁-C₆-alkoxy” refers to straight-chain or branched saturated alkyl groups comprising 1 to 6 carbon atoms, which groups are attached via an oxygen atom. Examples include C₁-C₆-alkoxy, such as, for example, methoxy, ethoxy, OCH₂—C₂H₅, OCH(CH₃)₂, n-butoxy, OCH(CH₃)—C₂H₅, OCH₂—CH(CH₃)₂ and OC(CH₃)₃.

The term “C₁-C₆-haloalkyl”, as used herein and in the haloalkyl moieties of C₁-C₆-haloalkoxy and C₁-C₆-haloalkylthio, refers to straight-chain or branched alkyl groups having 1 to 6 carbon atoms, wherein some or all of the hydrogen atoms of these groups are replaced by halogen atoms, such as chloromethyl, bromomethyl, dichloromethyl, trichloromethyl, fluoromethyl, difluoromethyl, trifluoromethyl, 1-chloroethyl, 1-bromoethyl, 1-fluoroethyl, 2-fluoroethyl, 2,2-difluoroethyl, 2,2,2-trifluoroethyl, 2,2,2-trichloroethyl, pentafluoroethyl, heptafluoroisopropyl, etc.

The term “C₂-C₆-haloalkenyl” as used herein, which is also expressed as “C₁-C₆-alkenyl which is partially or fully halogenated”, and the haloalkenyl moieties in haloalkenyloxy, haloalkenylcarbonyl and the like refers to unsaturated straight-chain or branched hydrocarbon radicals 2 to 6 carbon atoms and a double bond in any position (as mentioned above), wherein some or all of the hydrogen atoms in these groups are replaced by halogen atoms as mentioned above, in particular fluorine, chlorine and bromine, for example chlorovinyl, chloroallyl and the like.

The term “C₂-C₆-haloalkynyl” as used herein, which is also expressed as “C₁-C₆-alkynyl which is partially or fully halogenated”, and the haloalkynyl moieties in haloalkynyloxy, haloalkynylcarbonyl and the like refers to unsaturated straight-chain or branched hydrocarbon radicals having 2 to 6 (“C₂-C₆-haloalkynyl”) carbon atoms and one or two triple bonds in any position (as mentioned above), wherein some or all of the hydrogen atoms in these groups are replaced by halogen atoms as mentioned above, in particular fluorine, chlorine and bromine. The term “C₁-C₆-haloalkoxy” refers to C₁-C₆-haloalkyl groups, as defined above, which are attached via an oxygen atom. Examples include mono-, di- and trifluoromethoxy, mono-, di- and trichloromethoxy, 2,2,2-trifluoroethoxy, or heptafluoroisopropoxy.

The term “C₃-C₈-cycloalkyl”, as used herein, describes cyclic hydrocarbon radicals comprising 3 to 8 carbon atoms. Examples of cyclic radicals are cyclopropyl, cyclobutyl, cyclopentyl, cyclohexyl or cycloheptyl.

The term “C₃-C₈-halocycloalkyl” as used herein, which is also expressed as “C₃-C₈-cycloalkyl which is partially or fully halogenated”, and the halocycloalkyl moieties in halocycloalkoxy, halocycloalkylcarbonyl and the like refers to mono- or bi- or polycyclic saturated hydrocarbon groups having 3 to 8 (“C₃-C₈-halocycloalkyl”) carbon ring members (as mentioned above) in which some or all of the hydrogen atoms are replaced by halogen atoms as mentioned above, in particular fluorine, chlorine and bromine.

The term “carbocycle” or “carbocyclyl” includes, unless otherwise indicated, in general a 3- to 12-membered, preferably a 3- to 8-membered or a 5- to 8-membered, more preferably a 5- or 6-membered mono-cyclic, non-aromatic ring comprising 3 to 12, preferably 3 to 8 or 5 to 8, more preferably 5 or 6 carbon atoms. Preferably, the term “carbocycle” covers cycloalkyl and cycloalkenyl groups as defined above, for example cyclopropane, cyclobutane, cyclopentane and cyclohexane rings.

The term “heterocycle” or “heterocyclyl” includes, unless otherwise indicated, in general 3- to 12-membered, preferably 3- to 8-membered, 3- to 7-membered, or 5- to 8-membered, more preferably 5- or 6-membered, in particular 6-membered monocyclic heterocyclic non-aromatic radicals. The heterocyclic non-aromatic radicals usually comprise 1, 2, 3, 4 or 5, preferably 1, 2 or 3 heteroatoms selected from N, O and S as ring members, where S-atoms as ring members may be present as S, SO or SO₂. Examples of 5- or 6-membered heterocyclic radicals comprise saturated, or unsaturated, non-aromatic heterocyclic rings, such as oxiranyl, oxetanyl, thietanyl, thietanyl-S-oxide (S-oxothietanyl), thietanyl-S-dioxide (S-dioxothiethanyl), pyrrolidinyl, pyrrolinyl, pyrazolinyl, tetrahydrofuranyl, dihydrofuranyl, 1,3-dioxolanyl, thiolanyl, S-oxothiolanyl, S-dioxothiolanyl, dihydrothienyl, S-oxodihydrothienyl, S-dioxodihydrothienyl, oxazolidinyl, oxazolinyl, thiazolinyl, oxathiolanyl, piperidinyl, piperazinyl, pyranyl, dihydropyranyl, tetrahydropyranyl, 1,3- and 1,4-dioxanyl, thiopyranyl, S.oxothiopyranyl, S-dioxothiopyranyl, dihydrothiopyranyl, S-oxodihydrothiopyranyl, S-dioxodihydrothiopyranyl, tetrahydrothiopyranyl, S-oxotetrahydrothiopyranyl, S-dioxotetrahydrothiopyranyl, morpholinyl, thiomorpholinyl, S-oxothiomorpholinyl, S-dioxothiomorpholinyl, thiazinyl and the like. Examples for heterocyclic ring also comprising 1 or 2 carbonyl groups as ring members comprise pyrrolidin-2-onyl, pyrrolidin-2,5-dionyl, imidazolidin-2-onyl, oxazolidin-2-onyl, thiazolidin-2-onyl and the like.

The substituent “OTf” refers to a triflate substituent (trifluoromethane sulfonate), which is bonded to the rest of the molecule by a single bond to one oxygen atom of the sulfonic acid moiety.

The substituent “OTs” refers to a tosylate substituent (p-toluenesulfonate), which is bonded to the rest of the molecule by a single bond to one oxygen atom of the sulfonic acid moiety.

The term “substituted” refers in each case to a substitution by one, or more, same or different substituents.

If not otherwise stated, the preferred definitions of the different substituents relate to all compounds and processes where these are applicable. Combinations of embodiments with other embodiments, independent of their nature or preference, are within the scope of the invention.

In one embodiment, the substituents and indices in formula I have the following meaning:

X halogen;
R¹ OR¹¹ or NR¹²R¹³;
R¹¹ a) H;
   b) C₁-C₆-alkyl, C₃-C₈-cycloalkyl, C₂-C₆-alkenyl, C₃-C₈-cycloalkenyl, C₂-C₆-alkynyl; which groups are unsubstituted, or substituted by halogen, CN, NO₂, S(O)ₘR^{A}, OR^{B}, NR^{B}R^{C}, S(O)ₘNR^{B}R^{C}, Si(R^{B})₂R^{C}, C(═O)R^{B}, C(═O)NR^{B}R^{C}, C(═O)OR^{B}, C(═S)R^{B}, C(═S)NR^{B}R^{C}, C(═S)OR^{B}, C(═S)SR^{B}, C(═NR^{B})R^{C}, or C(═NR^{B})NR^{C}R^{D}
   c) phenyl, which is unsubstituted, or substituted by R^{A}; or
   d) a 3-, 4-, 5-, 6- or 7-membered saturated, partially unsaturated, or fully unsaturated heterocycle, which ring comprises one, or more, same, or different heteroatoms O, N(O)ₙ, and S(O)ₘ;
   wherein
   R^{A} a) C₁-C₆-alkyl, C₃-C₈-cycloalkyl-C₁-C₄-alkyl, C₁-C₄-alkyl-C₃-C₈-cycloalkyl, C₃-C₈-cycloalkyl, C₂-C₆-alkenyl, C₂-C₆-alkynyl, phenyl;
      which groups are unsubstituted, or partially, or fully substituted by halogen, CN, OH, NO₂;
      b) a 3-, 4-, 5-, 6- or 7-membered saturated, partially unsaturated, or fully unsaturated heterocycle, which ring comprises one, or more, same, or different heteroatoms O, N(O)ₙ, and S(O)ₘ;
   R^{B}, R^{C}, R^{D} have a meaning, independently from one another, as defined for R^{A}, or H;
R¹² H, C₁-C₆-alkyl, C₂-C₆-alkenyl, C₂-C₆-alkynyl, C₃-C₈-cycloalkyl, phenyl; which groups are unsubstituted, or substituted by R^{F};
R¹³ a) H, C₁-C₆-alkyl, C₂-C₆-alkenyl, C₂-C₆-alkynyl, C₃-C₈-cycloalkyl, phenyl; which groups are unsubstituted, or substituted by R^{F};
   b) a group Z-A, wherein Z is a chemical bond, CH₂, CH₂CH₂ or C═O; and A is a 3-, 4-, 5-, 6- or 7-membered saturated, partially unsaturated, or fully unsaturated heterocycle, which ring is unsubstituted, or substituted with one, or more, same, or different substituents R^{F} and comprises one, or more, same, or different heteroatoms O, N(O)ₙ, and S(O)ₘ;
   c) a group S(O)ₘR^{A}, S(O)ₙN(R^{B})R^{C}, N(R^{B})R^{C}, C(═O)N(R^{B})R^{C}, C(═S)N(R^{B})R^{C}, C(═O)OR^{A}, C═NOR^{A}, C═NR^{A}R^{B}, C═NR^{B}R^{C};
R^{F} a) halogen, CN, N₃, NO₂, SCN, SF₅, C₁-C₆-alkyl, C₁-C₆-haloalkyl, C₁-C₆-alkoxy-C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, Si(R^{B})₂R^{C}, OR¹¹, OSO₂R^{A}, S(O)ₘR^{A}, S(O)ₙN(R^{B})R^{C}, N(R^{B})R^{C}, C(═O)N(R^{B})R^{C},
   C(═S)N(R^{B})R^{C}, C(═O)OR^{A};
   b) phenyl, which is unsubstituted, or substituted by R^{A};
k 1 or 2
m 0, 1, or 2; and
n 0 or 1.

In one embodiment, k is 1. In another embodiment k is 2.

X is usually a halogen, preferably Cl or Br, more preferably Br. In one embodiment, X is Cl, Br, or I. In another embodiment, X is Br or I. In another embodiment, X is Cl. In another embodiment, X is I.

U is usually a halogen, preferably Cl or Br, more preferably Cl. In another embodiment, U is Cl, Br, or I. In yet another embodiment, U is Br, or I. In yet another embodiment, U is Br. In yet another embodiment, U is I.

In one embodiment, V is N, and W is CH₂. In another embodiment, V is N, and W is O. In another embodiment, W is CH₂ and V is CH. In another embodiment, W is O and V is CH. In another embodiment, W is S and V is CH. In another embodiment, W is S and V is N.

In one embodiment, R¹ is H.

In another embodiment, R¹ is OR¹¹ and R¹¹ is not H (ester form). The ester form is advantageously utilized in the production steps described herein due to higher yields, and less side reactions. Finally, protective groups are usually not required for the ester form.

R¹¹ is preferably R¹¹a), b), or c), more preferably R¹¹a) or R¹¹b). More preferably, R¹¹ is R¹¹a). Also more preferably, R¹¹ is R¹¹b), especially preferably C₁-C₆-alkyl, C₃-C₈-cycloalkyl, C₂-C₆-alkenyl, C₃-C₈-cycloalkenyl, C₂-C₆-alkynyl; which groups are unsubstituted, or substituted by selected from halogen, CN, NO₂, S(O)ₘR^{A}, OR^{B}, NR^{B}R^{C}, S(O)ₘNR^{B}R^{C}, Si(R^{B})₂R^{C}, C(═O)R^{B}, C(═O)NR^{B}R^{C}, C(═O)OR^{B}, C(═S)R^{B}, C(═S)NR^{B}R^{C}, C(═S)OR^{B}, C(═S)SR^{B}, C(═NR^{B})R^{C}, C(═NR^{B})NR^{C}R^{D}.

In particular, R¹¹ is C₁-C₆-alkyl or C₃-C₈-cycloalkyl; which groups are unsubstituted, or substituted by selected from halogen, CN, NO₂, S(O)ₘR^{A}, OR^{B}, NR^{B}R^{C}, C(═O)R^{B}, C(═O)NR^{B}R^{C}, C(═O)OR^{B}.

In one embodiment, R¹¹ is C₁-C₆-alkyl or C₃-C₈-cycloalkyl, preferably C₁-C₆-alkyl, more preferably C₁-C₄ alkyl (such as CH₃, CH₂CH₃, CH₂CH₂CH₃, CH(CH₃)₂, C(CH₃)₃), preferably CH₃.

In another embodiment, R¹¹ is C₁-C₆-alkyl, which is fully substituted with halogen, such as CF₃, CF₂CF₃, CF(CF₃)₂, CCl₃, CCl₂CCl₃, CCl(CCl₃)₂. In yet another embodiment, R¹¹ is C₁-C₆-alkyl, which is partially substituted with CN, NO₂, or OR^{B}, such as CH₂CN, CH₂CH₂CN, CH₂NO₂, CH₂CH₂NO₂, CH₂OH, CH₂CH₂OH, CH(OH)CH₃, CH₂OCH₃, CH₂OCH₂CH₃, CH₂OCH(CH₃)₂, CH₂OC(CH₃)₃, CH₂CH₂OCH₃, CH₂CH₂OCH₂CH₃, CH₂CH₂OCH(CH₃)₂, CH₂CH₂OC(CH₃)₃, CF₂OCF₃, CF₂OCF₂CF₃, CF₂OCF(CF₃)₂, CF₂OC(CF₃)₃, CF₂CF₂OCF₃, CF₂CF₂OCF₂CF₃, CF₂CF₂OCF(CF₃)₂, CF₂CF₂OC(CF₃)₃.

In one embodiment, R¹¹ is H, C₁-C₆-alkyl or C₃-C₈-cycloalkyl, preferably H, C₁-C₆-alkyl, more preferably H, C₁-C₄ alkyl (such as CH₃, CH₂CH₃, CH₂CH₂CH₃, CH(CH₃)₂, C(CH₃)₃). In another embodiment, R¹¹ is C₁-C₄-alkyl.

In one embodiment, R¹¹ is H, C₁-C₄ alkyl, preferably H, or CH₃, in particular CH₃.

In another embodiment, R¹¹ is C₁-C₆-alkyl, which is fully substituted with halogen, such as CF₃, CF₂CF₃, CF(CF₃)₂, CCl₃, CCl₂CCl₃, CCl(CCl₃)₂.

In yet another embodiment, R¹¹ is C₁-C₆-alkyl, which is partially substituted with CN, NO₂, or OR^{B}, such as CH₂CN, CH₂CH₂CN, CH₂NO₂, CH₂CH₂NO₂, CH₂OH, CH₂CH₂OH, CH(OH)CH₃, CH₂OCH₃, CH₂OCH₂CH₃, CH₂OCH(CH₃)₂, CH₂OC(CH₃)₃, CH₂CH₂OCH₃, CH₂CH₂OCH₂CH₃, CH₂CH₂OCH(CH₃)₂, CH₂CH₂OC(CH₃)₃, CF₂OCF₃, CF₂OCF₂CF₃, CF₂OCF(CF₃)₂, CF₂OC(CF₃)₃, CF₂CF₂OCF₃, CF₂CF₂OCF₂CF₃, CF₂CF₂OCF(CF₃)₂, CF₂CF₂OC(CF₃)₃.

Also more preferably, R¹¹ is R¹¹c) or R¹¹d), especially preferably unsubstituted phenyl, or a 3-, 4-, 5, or 6-membered saturated, or fully unsaturated heterocycle, which heterocycle comprises one, or more, same, or different heteroatoms O, N(O)ₙ, and S(O)ₘ, and in particular phenyl.

In one embodiment, R¹ is OR¹¹ and R¹¹ is H, C₁-C₆-alkyl, C₃-C₈-cycloalkyl, C₂-C₆-alkenyl, C₃-C₈-cycloalkenyl, C₂-C₆-alkynyl; which groups are unsubstituted, or substituted by selected from halogen, CN, NO₂, S(O)ₘR^{A}, OR^{B}, NR^{B}R^{C}, S(O)ₘNR^{B}R^{C}, Si(R^{B})₂R^{C}, C(═O)R^{B}, C(═O)NR^{B}R^{C}, C(═O)OR^{B}, C(═S)R^{B}, C(═S)NR^{B}R^{C}, C(═S)OR^{B}, C(═S)SR^{B}, C(═NR^{B})R^{C}, C(═NR^{B})NR^{C}R^{D}.

In another embodiment, R¹ is OR¹¹ and R¹¹ is H, C₁-C₆-alkyl, preferably H or CH₃, more preferably CH₃. In another embodiment, R¹ is OR¹¹ and R¹¹ is H, C₁-C₆-alkyl, C₃-C₈-cycloalkyl, C₂-C₆-alkenyl, C₃-C₈-cycloalkenyl, C₂-C₆-alkynyl, especially preferably C₁-C₆-alkyl. In yet another embodiment, R¹¹ is H, C₁-C₆-alkyl, phenyl, or benzyl. In yet another embodiment, R¹¹ is C₁-C₆-alkyl, phenyl, or benzyl.

R^{A} is usually C₁-C₆-alkyl, C₃-C₈-cycloalkyl-C₁-C₄-alkyl, C₁-C₄-alkyl-C₃-C₈-cycloalkyl, C₃-C₈-cycloalkyl, C₂-C₆-alkenyl, C₂-C₆-alkynyl, phenyl; which groups are unsubstituted, or partially, or fully substituted by halogen, CN, OH, NO₂. Preferably, R^{A} is C₁-C₆-alkyl, C₃-C₈-cycloalkyl-C₁-C₄-alkyl, C₁-C₄-alkyl-C₃-C₈-cycloalkyl, C₃-C₈-cycloalkyl, C₂-C₆-alkenyl, C₂-C₆-alkynyl, phenyl, more preferably C₁-C₆-alkyl.

In another embodiment of the invention, R¹ is NR¹²R¹³.

Preferably, R¹² is H, CH₃, CH₂CH₃, CH₂CH₂CH₃, C(═O)CH₃, or C(═O)OCH₃, more preferably H, CH₃, CH₂CH₃, or CH₂CH₂CH₃, even more preferably H or CH₃, and most preferably H. In another embodiment, R¹² is H, or C₁-C₆-alkyl.

In one embodiment, R¹³ is H, C₁-C₆-alkyl, C₂-C₆-alkenyl, C₂-C₆-alkynyl, C₃-C₈-cycloalkyl, phenyl, preferably CH₃, CH₂CH₃, CH₂CH₂CH₃, CH(CH₃)₂, CH₂CH₂CH₂CH₃, CH(CH₃)CH₂CH₃, C(CH₃)₃, CHCH₂, CH₂CHCH₂, CHCHCH₃, CCH, CH₂CCH, isopropyl, isobutyl, isopentyl, isohexyl, phenyl, more preferably CH₃, CH₂CH₃, CH₂CH₂CH₃, CH(CH₃)₂, CH₂CH₂CH₂CH₃, CH(CH₃)CH₂CH₃, C(CH₃)₃, CH(CH₃)₂, CH₂CH(CH₃)₂, (CH₂)₂CH(CH₃)₂, (CH₂)₃CH(CH₃)₂, phenyl.

In another embodiment, R¹³ is C₁-C₆-alkyl, C₂-C₆-alkenyl, C₂-C₆-alkynyl, C₃-C₈-cycloalkyl, phenyl, which groups are fully substituted with R^{F}. In another embodiment, R¹³ is C₁-C₆-alkyl, C₂-C₆-alkenyl, C₂-C₆-alkynyl, C₃-C₈-cycloalkyl, phenyl, which groups are partially (e.g. 1, 2, 3, 4, 5 times) substituted with R^{F}.

In another embodiment, R¹³ is C₁-C₆-alkyl, C₂-C₆-alkenyl, C₃-C₈-cycloalkyl, which groups are partially (e.g. 1, 2, 3, 4, 5 times) substituted with halogen or C₃-C₈-cycloalkyl.

In another embodiment, R¹³ is C₁-C₆-alkyl, C₃-C₈-cycloalkyl, which groups are partially (e.g. 1, 2, 3, 4, 5 times) substituted with R^{F}, preferably with halogen, or S(O)ₘR^{A}.

In another embodiment, R¹³ is C₁-C₆-alkyl, which is partially substituted with halogen or C₃-C₈-cycloalkyl, preferably with C₃-C₈-cycloalkyl.

In another embodiment, R¹³ is a) H, C₁-C₆-alkyl, C₁-C₆-alkoxy, C₂-C₆-alkenyl, C₂-C₆-alkynyl, C₃-C₈-cycloalkyl, C₃-C₈-cycloalkyl-C₁-C₆-alkyl, phenyl; which groups are unsubstituted, or substituted by R^{E};

b) a group Z-A, wherein Z is a chemical bond, CH₂, CH₂CH₂ or C═O; and A is a 3-, 4-, 5-, 6- or 7-membered saturated, partially unsaturated, or fully unsaturated heterocycle, which heterocycle is unsubstituted, or substituted with one, or more, same, or different substituents R^{F} and comprises one, or more, same, or different heteroatoms O, N(O)ₙ, and S(O)ₘ, and wherein none, one, or more ring members are replaced by C(═O), or C(═S), C(═NR^{B}), or C(═NOR^{B});
c) a group S(O)ₘR^{A}, S(O)ₘN(R^{B})R^{C}, N(R^{B})R^{C}, N(R^{B})C(═O)OR^{C}, N(R^{B})C(═O)N(R^{C})R^{D}, C(═O)N(R^{B})R^{C}, C(═O)OR^{A}, C═NOR^{A}, C═NR^{A}R^{B};
or wherein R¹² and R¹³, together with the N-atom to which they are bound, form a 3, 4, 5, 6, or 7-membered saturated, partially unsaturated, or fully unsaturated carbocycle, or heterocycle, which cycles are unsubstituted, or substituted with one, or more, same, or different substituents R^{F}, and wherein the heterocycle comprises one, or more, same, or different heteroatoms O, N(O)ₙ, and S(O)ₘ, and wherein none, one, or more ring members are replaced by C(═O);
or wherein R¹² and R¹³, together with the N-atom to which they are bound, form a group ═S(R^{B})R^{C}.

In another embodiment, R¹³ is a) H, C₁-C₆-alkyl, C₁-C₆-alkoxy, C₂-C₆-alkenyl, C₂-C₆-alkynyl, C₃-C₈-cycloalkyl, C₃-C₈-cycloalkyl-C₁-C₆-alkyl, phenyl; which groups are unsubstituted, or substituted by R^{E};

b) a group Z-A, wherein Z is a chemical bond, CH₂, CH₂CH₂ or C═O; and A is selected from A-1 to A-31;
c) a group S(O)ₘR^{A}, S(O)ₘN(R^{B})R^{C}, N(R^{B})R^{C}, N(R^{B})C(═O)OR^{C}, N(R^{B})C(═O)N(R^{C})R^{D}, C(═O)N(R^{B})R^{C}, C(═O)OR^{A}, C═NOR^{A}, C═NR^{A}R^{B};
or wherein R¹² and R¹³, together with the N-atom to which they are bound, form a 3, 4, 5, 6, or 7-membered saturated, partially unsaturated, or fully unsaturated heterocycle, which heterocycle is unsubstituted, or substituted with one, or more, same, or different substituents R^{F} and comprises one, or more, same, or different heteroatoms O, N(O)ₙ, and S(O)ₘ, and wherein none, one, or more ring members are replaced by C(═O);
or wherein R¹² and R¹³, together with the N-atom to which they are bound, form a group ═S(R^{B})R^{C}.

In another embodiment, R¹³ is a) H, C₁-C₆-alkyl, C₁-C₆-alkoxy, C₂-C₆-alkenyl, C₂-C₆-alkynyl, C₃-C₈-cycloalkyl, C₃-C₈-cycloalkyl-C₁-C₆-alkyl, phenyl; which groups are unsubstituted, or substituted by R^{E};

b) a group Z-A, wherein Z is a chemical bond, CH₂, or CH₂CH₂; and A is selected from A-1 to A-31;
c) a group S(O)ₘR^{A}, S(O)ₘN(R^{B})R^{C}, N(R^{B})R^{C}, N(R^{B})C(═O)OR^{C}, N(R^{B})C(═O)N(R^{C})R^{D}, C(═O)N(R^{B})R^{C}, C(═O)OR^{A}, C═NOR^{A}, C═NR^{A}R^{B};
or wherein R¹² and R¹³, together with the N-atom to which they are bound, form a 3, 4, 5, 6, or 7-membered saturated, partially unsaturated, or fully unsaturated heterocycle, which heterocycle is unsubstituted, or substituted with one, or more, same, or different substituents R^{F} and comprises one, or more, same, or different heteroatoms O, N(O)ₙ, and S(O)ₘ, and wherein none, one, or more ring members are replaced by C(═O);
or wherein R¹² and R¹³, together with the N-atom to which they are bound, form a group ═S(R^{B})R^{C}.

In another embodiment, R¹³ is
a) H;
b) C₁-C₆-alkyl, C₂-C₆-alkynyl; which groups are unsubstituted, or substituted by R^{E};
c) C₃-C₈-cycloalkyl, C₃-C₈-cycloalkyl-C₁-C₆-alkyl; which groups are unsubstituted, or substituted by R^{E}, C₁-C₂-alkyl, or C₁-C₂-haloalkyl;
d) a group Z-A, wherein Z is a chemical bond, CH₂, or CH₂CH₂;
   wherein A is a 3-, 4-, 5-, 6- or 7-membered saturated, partially unsaturated, or fully unsaturated heterocycle, which heterocycle is unsubstituted, or substituted with one, or more, same, or different substituents R^{F} and comprises one, or more, same, or different heteroatoms O, N(O)ₙ, and S(O)ₘ, and wherein none, one, or more ring members are replaced by C(═O), or C(═S);

In one embodiment, R^{F} is halogen, CN, NO₂, C₁-C₆-alkyl, C₁-C₆-haloalkyl, C₁-C₆-alkoxy-C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, S(O)ₘR^{A}, C(═O)OR^{A}, phenyl, or pentafluorophenyl, more preferably halogen, CN, C₁-C₆-alkyl, C₁-C₆-haloalkyl, C₂-C₆-alkenyl, C₂-C₆-haloalkenyl, C₂-C₆-alkynyl, C₂-C₆-haloalkynyl, most preferably halogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, and especially preferably halogen.

In another embodiment, R^{F} is a) halogen, CN, N₃, NO₂, C₁-C₆-alkyl, C₁-C₆-haloalkyl, C₁-C₆-alkoxy-C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, OR¹¹, S(O)ₘR^{A}, S(O)ₘN(R^{B})R^{C}, N(R^{B})R^{C}, C(═O)N(R^{B})R^{C}, C(═O)N(R^{B})N(R^{C})R^{D}, C(═O)NOR^{B}, N(R^{B})R^{C}, C(═O)OR^{A};

b) phenyl, which is unsubstituted, or substituted by R^{A}; or
c) two substituents R^{F}, together with the atom, or the atoms to which they are bound, form a 3, 4, 5, 6, or 7-membered saturated, partially unsaturated, or fully unsaturated carbocycle, or heterocycle, which cycles are unsubstituted, or substituted with one, or more, same, or different substituents R^{A} and wherein the heterocycle comprises one, or more, same, or different heteroatoms O, N(O)ₙ, and S(O)ₘ, and wherein none, one, or more ring members are replaced by C(═O), or C(═S), C(═NR^{B}), or C(═NOR^{B});

In yet another embodiment, R¹³ is a group Z-A, wherein Z is a chemical bond, CH₂, CH₂CH₂ or C═O; and A is a 3-, 4-, 5-, 6- or 7-membered saturated, partially unsaturated, or fully unsaturated heterocycle, which heterocyle is unsubstituted, or substituted with one, or more, same, or different substituents R^{F} and comprises one, or more, same, or different heteroatoms O, N(O)ₙ, and S(O)ₘ.

Preferably, A is selected from

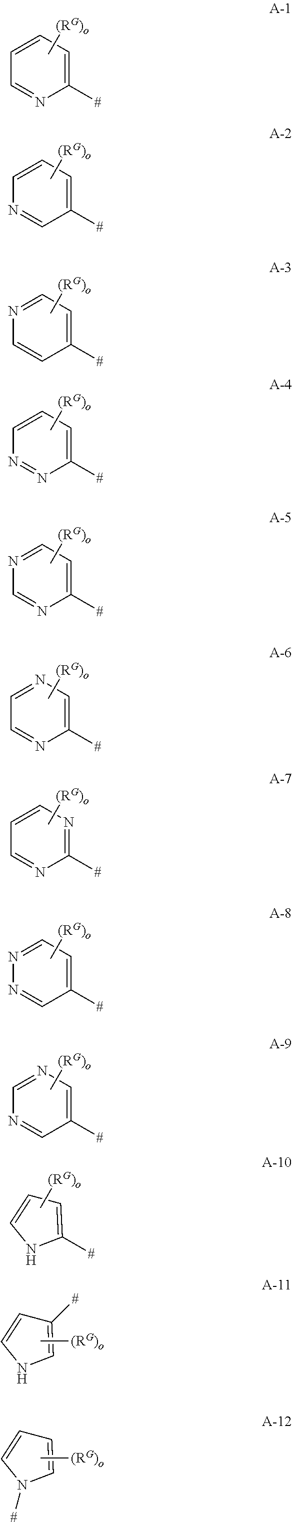

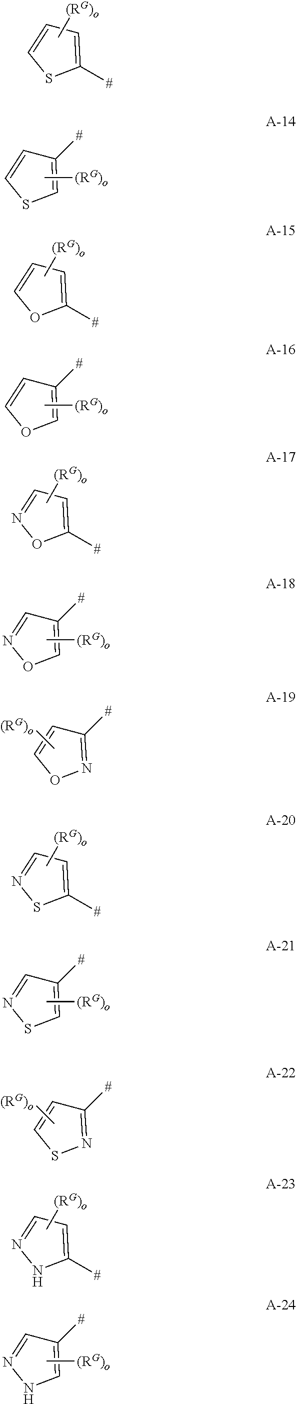

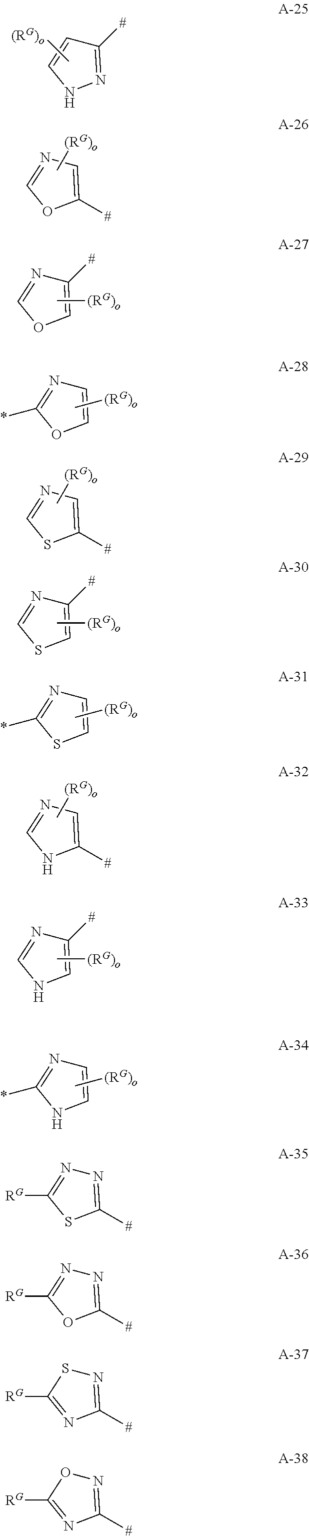

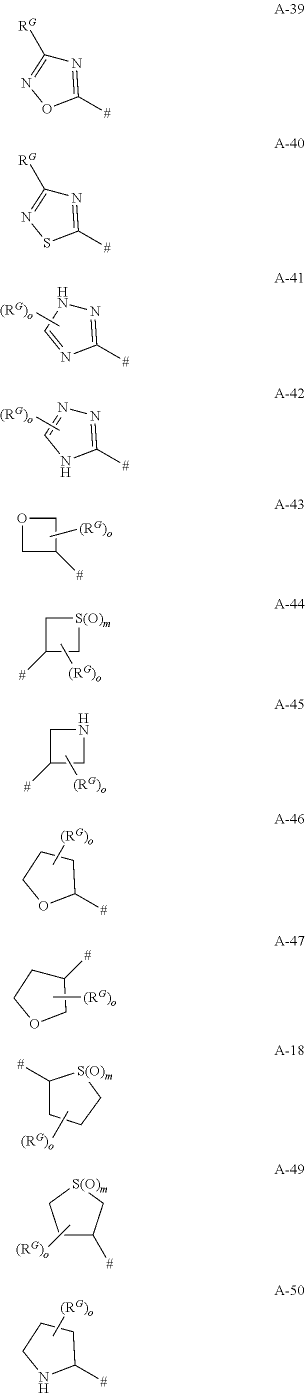

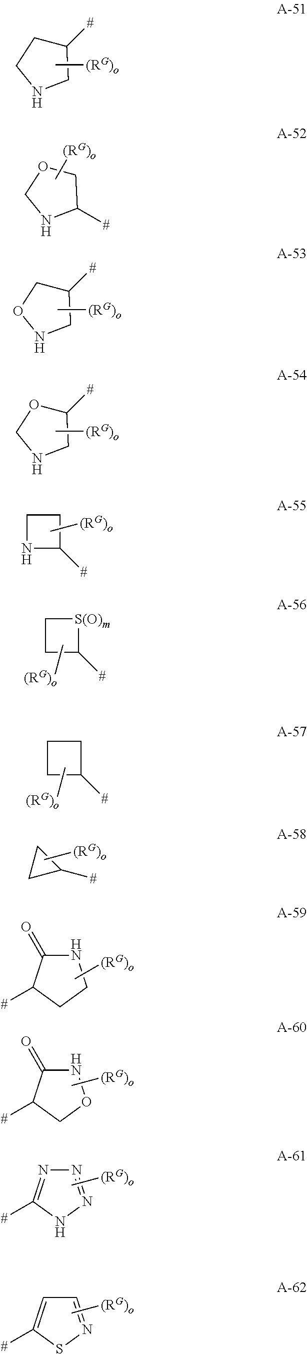

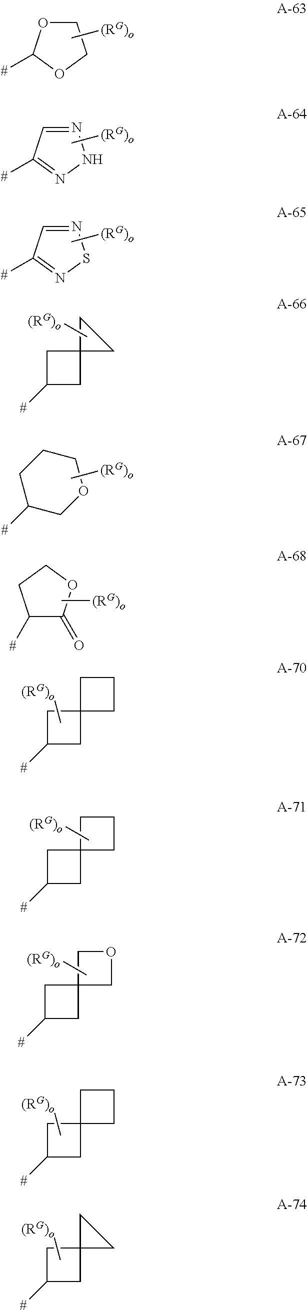

wherein # stands for the connection to Z. In case of A-59 and A-60, the chirality center marked with an asterisk usually has two stable stereoisomers, which may be supplied in their isolated, or racemic form. In one embodiment, A-59 and/or A-60 are supplied as the S-diastereomer (or enantiomer). In another embodiment, A-59 and/or A-60 are supplied as the R-diastereomer (or enantiomer). For the avoidance of doubt, the chirality center is localized at the carbon that is directly linked to Z by a single bond. In one embodiment, A is selected from A-1 to A-60.

Substituents R^{G} are usually same or different substituents selected from halogen, CN, NO₂, C₁-C₆-alkyl, N(C₁-C₆-alkyl)(C₁-C₆-alkyl), C₁-C₆-haloalkyl, C₁-C₆-alkoxy, C₁-C₆-haloalkoxy, C₁-C₆-alkyl-S(O)ₘ, C₁-C₆-haloalkyl-S(O)ₘ, C₃-C₆-cycloalkyl, C₃-C₆-halocycloalkyl, C₂-C₆-alkenyl, C₂-C₆-haloalkenyl, C₂-C₆-alkynyl, C₂-C₆-haloalkynyl, C₁-C₄-alkyl-C(O), C₁-C₄-haloalkyl-C(O), C(═O)NR^{B}R^{C}; or two R^{G} present on the same carbon atom of a saturated ring may form together ═O or ═S.

In one embodiment, R^{E} are same or different substituents selected from halogen, CN, NO₂, C₁-C₆-alkyl, C₁-C₆-haloalkyl, C₁-C₆-alkoxy, C₁-C₆-haloalkoxy, C₁-C₆-alkyl-S(O)ₘ, C₁-C₆-haloalkyl-S(O)ₘ, C₃-C₆-cycloalkyl, C₃-C₆-halocycloalkyl, C₂-C₆-alkenyl, C₂-C₆-haloalkenyl, C₂-C₆-alkynyl, C₂-C₆-haloalkynyl, C₁-C₄-alkyl-C(O), C₁-C₄-haloalkyl-C(O), C(═O)NR^{B}R^{C}; or two R^{E} present on the same carbon atom of a saturated ring may form together ═O or ═S.

In one embodiment, R^{G} is halogen, CN, C₁-C₄-alkyl, C₁-C₄-haloalkyl, C₁-C₄-alkoxy, C₁-C₄-haloalkoxy, C₁-C₄-alkyl-S(O)ₘ, C₁-C₄-haloalkyl-S(O)ₘ, C₃-C₆-cycloalkyl, C₃-C₆-halocycloalkyl, C₂-C₄-alkenyl, C₂-C₄-haloalkenyl, C₂-C₄-alkynyl, C₂-C₄-haloalkynyl, more preferably halogen, CN, C₁-C₄-alkyl, most preferably halogen or C₁-C₄-alkyl.

The index o is usually 0, 1, or 2, preferably 0 or 1. In one embodiment, the index o is 0. In another embodiment, the index o is 1.

In one embodiment, A is selected from A-1 to A-9. In another embodiment, A is selected from A-10 to A-16. In yet another embodiment, A is selected from A-17 to A-42. In yet another embodiment, A is selected from A-43 to A-60, preferably A-55 to A-60. In yet another embodiment, A is selected from A-1, A-7, A-56, and A-58. In yet another embodiment, A is selected from A-59 and A-60. In yet another embodiment, A is selected from A-1, A-7, and A-56 to A-60. In yet another example, A is selected from A-7 and A-56. In yet another embodiment, A is selected from A-1, A-4 to A-7. In yet another embodiment, A is selected from A-1, A-4 to A-7, A-10, A-19, A-22, A-23, A-25, A-27, A-28, A-30, A-31, A-32 to A-42, A-50, A-52, and A-55.

Preferably, Z is a chemical bond, CH₂, or CH₂CH₂, more preferably a chemical bond, or CH₂.

In one embodiment, R¹³ is a group S(O)ₘR^{A}, S(O)ₙN(R^{B})R^{C}, N(R^{B})R^{C}, C(═O)N(R^{B})R^{C}, C(═S)N(R^{B})R^{C}, C(═O)OR^{A}, C═NOR^{A}, C═NR^{A}R^{B}, C═NR^{B}R^{C}, preferably S(O)ₘR^{A}, S(O)ₙN(R^{B})R^{C}, N(R^{B})R^{C}, C(═O)N(R^{B})R^{C}, C═NOR^{A}, C═NR^{A}R^{B}, C═NR^{B}R^{C}. In another embodiment, R¹³ is a group C═NOR^{A}, C═NR^{A}R^{B}, C═NR^{B}R^{C}, preferably C═NOR^{A}. In another embodiment, R¹³ is a group N(R^{B})R^{C}, C(═O)N(R^{B})R^{C}, C(═S)N(R^{B})R^{C}, C═NOR^{A}, C═NR^{A}R^{B}, C═NR^{B}R^{C}, preferably N(R^{B})R^{C}, C═NOR^{A}, C═NR^{A}R^{B}, C═NR^{B}R^{C}.

In one embodiment, R¹ is NR¹²R¹³, R¹² is H or CH₃, R¹³ is C₁-C₆-alkyl, C₂-C₆-alkenyl, C₂-C₆-alkynyl, C₃-C₈-cycloalkyl, phenyl.

In another embodiment, R¹ is NR¹²R¹³, R¹² is H, R¹³ is CH₃, CH₂CH₃, CH₂CH₂CH₃, CH(CH₃)₂, CH₂CH₂CH₂CH₃, CH(CH₃)CH₂CH₃, C(CH₃)₃, isopropyl, isobutyl, isopentyl, isohexyl, phenyl, in particular CH₃, CH₂CH₃, phenyl.

In another embodiment, R¹ is NR¹²R¹³, R¹² is H or CH₃, R¹³ is C₁-C₆-alkyl, C₂-C₆-alkenyl, C₂-C₆-alkynyl, C₃-C₈-cycloalkyl, phenyl, which is fully substituted with R^{F},

wherein R^{F} is halogen, CN, NO₂, C₁-C₆-alkyl, C₁-C₆-haloalkyl, C₁-C₆-alkoxy-C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, S(O)ₘR^{A}, C(═O)OR^{A}, phenyl, or pentafluorophenyl.

In another embodiment, R¹ is NR¹²R¹³, R¹² is H, R¹³ is C₁-C₆-alkyl, C₂-C₆-alkenyl, C₂-C₆-alkynyl, C₃-C₈-cycloalkyl, phenyl, which is fully substituted with R^{F},

wherein R^{F} is halogen, CN, NO₂, C₁-C₆-alkyl, C₁-C₆-haloalkyl, C₁-C₆-alkoxy-C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, S(O)ₘR^{A}, C(═O)OR^{A}, phenyl, or pentafluorophenyl

In another embodiment, R¹ is NR¹²R¹³, R¹² is H or CH₃, R¹³ is C₁-C₆-alkyl, C₂-C₆-alkenyl, C₂-C₆-alkynyl, C₃-C₈-cycloalkyl, phenyl, which is once substituted with R^{F}, wherein R^{F} is halogen, CN, NO₂, C₁-C₆-alkyl, C₁-C₆-haloalkyl, C₁-C₆-alkoxy-C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, S(O)ₘR^{A}, C(═O)OR^{A}, phenyl, or pentafluorophenyl.

In another embodiment, R¹ is NR¹²R¹³, R¹² is H, R¹³ is C₁-C₆-alkyl, C₂-C₆-alkenyl, C₂-C₆-alkynyl, C₃-C₈-cycloalkyl, phenyl, which is once substituted with R^{F}, wherein R^{F} is halogen, CN, NO₂, C₁-C₆-alkyl, C₁-C₆-haloalkyl, C₁-C₆-alkoxy-C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, S(O)ₘR^{A}, C(═O)OR^{A}, phenyl, or pentafluorophenyl.

In another embodiment, R¹ is NR¹²R¹³, R¹² is H, R¹³ is C₁-C₆-alkyl, C₂-C₆-alkenyl, C₃-C₈-cycloalkyl, which groups are partially substituted with halogen, C₃-C₈-cycloalkyl.

In another embodiment, R¹ is NR¹²R¹³, R¹² is H, R¹³ is C₁-C₆-alkyl, which is partially substituted with halogen or C₃-C₈-cycloalkyl, preferably with C₃-C₈-cycloalkyl.

In another embodiment, R¹ is NR¹²R¹³, R¹² is H or CH₃, R¹³ is Z-A, Z is a chemical bond, CH₂, or CH₂CH₂, A is selected from A-10 to A-16, the index o is 0.

In another embodiment, R¹ is NR¹²R¹³, R¹² is H, R¹³ is Z-A, Z is a chemical bond, CH₂, or CH₂CH₂, A is selected from A-10 to A-16, the index o is 0.

In another embodiment, R¹ is NR¹²R¹³, R¹² is H, R¹³ is Z-A, Z is a chemical bond, CH₂, or CH₂CH₂, A is selected from A-10 to A-16, the index o is 1, 2, or 3, R^{G} is halogen, CN, C₁-C₄-alkyl.

In another embodiment, R¹ is NR¹²R¹³, R¹² is H or CH₃, R¹³ is Z-A, Z is a chemical bond, CH₂, or CH₂CH₂, A is selected from A-43 to A-60, preferably A-55 to A-60, the index o is 0.

In another embodiment, R¹ is NR¹²R¹³, R¹² is H, R¹³ is Z-A, Z is a chemical bond, CH₂, or CH₂CH₂, A is selected from A-43 to A-60, preferably A-55 to A-60, the index o is 0.

In another embodiment, R¹ is NR¹²R¹³, R¹² is H, R¹³ is Z-A, Z is a chemical bond, CH₂, or CH₂CH₂, A is selected from A-43 to A-60, preferably A-55 to A-60, and the index o is 1, 2, or 3, R^{G} is halogen, CN, C₁-C₄-alkyl.

In another embodiment, R¹ is NR¹²R¹³, R¹² is H or CH₃, R¹³ is Z-A, Z is a chemical bond, CH₂, or CH₂CH₂, A is selected from A-1, A-7, A-56, and A-58, and the index o is 0.

In another embodiment, R¹ is NR¹²R¹³, R¹² is H, R¹³ is Z-A, Z is a chemical bond, CH₂, or CH₂CH₂, A is selected from A-1, A-7, A-56, and A-58, and the index o is 0.

In another embodiment, R¹ is NR¹²R¹³, R¹² is H, R¹³ is Z-A, Z is a chemical bond, CH₂, or CH₂CH₂, A is selected from A-1, A-7, A-56, and A-58, and the index o is 1, 2, or 3, R^{G} is halogen, CN, C₁-C₄-alkyl.

In another embodiment, R¹ is NR¹²R¹³, R¹² is H or CH₃, R¹³ is Z-A, Z is a chemical bond, CH₂, or CH₂CH₂, A is selected from A-1, A-4 to A-7, the index o is 0.

In another embodiment, R¹ is NR¹²R¹³, R¹² is H, R¹³ is Z-A, Z is a chemical bond, CH₂, or CH₂CH₂, A is selected from A-1, A-4 to A-7, the index o is 0.

In another embodiment, R¹ is NR¹²R¹³, R¹² is H, R¹³ is Z-A, Z is a chemical bond, CH₂, or CH₂CH₂, A is selected from A-1, A-4 to A-7, the index o is 1, 2, or 3, R^{G} is halogen, CN, C₁-C₄-alkyl.

In another embodiment, R¹ is NR¹²R¹³, R¹² is H or CH₃, R¹³ is Z-A, Z is a chemical bond, CH₂, or CH₂CH₂, A is selected from A-1, A-4 to A-7, A-10, A-19, A-22, A-23, A-25, A-27, A-28, A-30, A-31, A-32 to A-42, A-50, A-52, and A-55, the index o is 0.

In another embodiment, R¹ is NR¹²R¹³, R¹² is H, R¹³ is Z-A, Z is a chemical bond, CH₂, or CH₂CH₂, A is selected from A-1, A-4 to A-7, A-10, A-19, A-22, A-23, A-25, A-27, A-28, A-30, A-31, A-32 to A-42, A-50, A-52, and A-55, the index o is 0.

In another embodiment, R¹ is NR¹²R¹³, R¹² is H, R¹³ is Z-A, Z is a chemical bond, CH₂, or CH₂CH₂, A is selected from A-1, A-4 to A-7, A-10, A-19, A-22, A-23, A-25, A-27, A-28, A-30, A-31, A-32 to A-42, A-50, A-52, and A-55, and the index o is 1, 2, or 3, R^{G} is halogen, CN, C₁-C₄-alkyl.

In another embodiment, R¹ is NR¹²R¹³, R¹² is H, R¹³ is Z-A, Z is a chemical bond, CH₂, or CH₂CH₂, A is selected from A-1, A-7, A-56, and A-58, the index o is 0.

In another embodiment, R¹ is NR¹²R¹³, R¹² is H or CH₃, R¹³ is Z-A, Z is a chemical bond, CH₂, or CH₂CH₂, A is selected from A-59 and A-60, the index o is 0.

In another embodiment, R¹ is NR¹²R¹³, R¹² is H, R¹³ is Z-A, Z is a chemical bond, A is selected from A-59 and A-60, the index o is 0.

In another embodiment, R¹ is NR¹²R¹³, R¹² is H, R¹³ is Z-A, Z is a chemical bond, A is selected from A-59 and A-60, the index o is 1, 2, or 3, R^{G} is halogen, CN, C₁-C₄-alkyl.

In another embodiment, R¹ is NR¹²R¹³, R¹² is H, R¹³ is Z-A, Z is a chemical bond, A is selected from A-59 and A-60, the index o is 1, R^{G} is C₁-C₄-alkyl.

In a preferred embodiment, R¹ is NR¹²R¹³, R¹² is H, R¹³ selected from is C₁-C₆-alkyl, C₂-C₆-alkenyl, C₃-C₈-cycloalkyl, which groups are unsubstituted, or substituted by R^{F}; wherein R^{F} is selected from halogen, C₃-C₈-cycloalkyl, S(O)ₘR^{A}; and wherein R¹³ is further selected from Z-A, wherein Z is a chemical bond, or CH₂, and wherein A is selected from A-1, A-7, A-56 to A-60; and wherein R¹³ is further selected from C═NOR^{A}.

In a preferred embodiment, R¹ is NR¹²R¹³, R¹² is H, R¹³ selected from is C₁-C₆-alkyl, C₂-C₆-alkenyl, C₃-C₈-cycloalkyl, which groups are unsubstituted, or substituted by R^{F}; wherein R^{F} is selected from halogen, C₃-C₈-cycloalkyl; and wherein R¹³ is further selected from Z-A, wherein Z is a chemical bond, or CH₂, and wherein A is selected from A-1, A-7, A-56 to A-60; and wherein R¹³ is further selected from C═NOR^{A}.

In another preferred embodiment, R¹ is NR¹²R¹³, R¹² is H, R¹³ selected from is C₁-C₆-alkyl, which is unsubstituted; and wherein R¹³ is further selected from Z-A, wherein Z is a chemical bond, or CH₂, and wherein A is selected from A-7 and A-56.

The following examples illustrate the invention.

### EXAMPLES

### I. Characterization

The characterization can be done by coupled High Performance Liquid Chromato-graphy/mass spectrometry (HPLC/MS), by NMR or by their melting points.

HPLC/MS. The gradient was 5-95% B in 0.7 min, 95-95% B in 0.45 min, 95-5% B in 0.01 min, and then hold at 0% B for 0.44 min (1.5 mL/min flow rate). Mobile phase A was 0.0375% TFA in water, mobile phase B was 0.018% TFA (trifluoroacetic acid) in MeCN (acetonitrile). Column temperature was 40° C. The column used for the chromatography was a Chromolith Flash RP-18e 25-2 mm column. MS-method: ESI positive.

NMR data of compounds and intermediates are summarized in Table 2.

HPLC-MS data of compounds and intermediates are summarized in Table 3 to 5. HPLC devices, solvents, columns, and gradients are listed in Table 7.

¹H-NMR: The signals are characterized by chemical shift (ppm) vs. tetramethylsilane, by their multiplicity and by their integral (relative number of hydrogen atoms given). The following abbreviations are used to characterize the multiplicity of the signals: m=multiplett, q=quartett, t=triplett, d=doublet and s=singlet.

Abbreviations used are: h for hour(s), min for minute(s), eq for equivalent(s).

### Preparation Examples

### Example 1: Production of ethyl (E)-3-(5-bromo-2-cyano-phenyl)prop-2-enoate (IVa.1)

A mixture of NaH (1.05 g/60 wt % in mineral oil) and DME (7 mL) was produced and cooled to −30° C. Subsequently, a solution of ethyl 2-diethoxyphosphorylacetate (5.9 g) in DME (47 mL) was added dropwise to the mixture over 15 minutes and stirred at −30° C. for further 35 minutes. Then, a solution of 4-bromo-2-formylbenzonitrile (5 g) in DME (43 mL) was added at about −30° C. dropwise over 20 minutes, and the mixture was stirred for additional 2 h. The reaction was quenched by addition of H₂O (70 mL), which was extracted with ethyl acetate. The organic extracts were combined and the solvent was evaporated. Compound IVa.1 was isolated by silica chromatography, resulting in a final yield of 88%. No impurities were detectable by H-NMR.

### Example 2: Production of 4-bromo-2-(2-carboxyethyl)benzoic acid (III.1)

A mixture of compound IVa (1 g) from Example 1 with 25 mL of acetic acid was produced. Subsequently Zn powder (2 g) was added and the mixture stirred for 3 h at 80° C. The mixture was filtrated and the filtrate concentrated by evaporation of the solvent. Ethyl 3-(5-bromo-2-cyano-phenyl)propanoate was obtained with a yield of 100%, no impurities were detectable by H-NMR.

In turn, a premix of H₂SO₄ (2.1 g) with 1 mL of H₂O and 2.6 mL acetic acid was produced. Ethyl 3-(5-bromo-2-cyano-phenyl)propanoate (400 mg) was added to the premix and the resulting reaction mixture was stirred for 20 h at about 139° C. The reaction was then cooled by addition of 25 g of ice. The precipitated compound III.1 was filtrated, washed with H₂O, and the resulting crystals were dried. The final yield was 75%.

### Example 3: Production of methyl 7-bromo-1-oxo-indane-4-carboxylate (II.1)

Step 1: A mixture of compound III.1 (600 mg) from Example 2 with 26 mL of CH₂Cl₂ and a few drops of DMF was produced. Oxalylchloride (1.9 g) was added dropwise at about 25° C. and the mixture was stirred for 35 minutes. The solvent and remaining oxalylchloride were evaporated and 4-bromo-2-(3-chloro-3-oxo-propyl)benzoyl chloride (compound III.1) was instantly further converted by Friedel-Crafts acylation.

Step 2: A mixture of AlCl₃ (469 mg) and CH₂Cl₂ (10 mL) was produced. A solution of compound III.1 (692 mg) in CH₂Cl₂ (20 mL) was added to the mixture at about 0° C. over 12 minutes. The mixture was then heated to about 40° C. and stirred under reflux.

Chemical shifts, multiplicity, and peak intensity for 7-bromo-1-oxo-indane-4-carbonyl chloride (compound V.1) are summarized in Table 2.

Compound V.1 was then esterified by addition of CH₃OH (10 mL) to the mixture at about 15° C. The solvent was evaporated, 5 mL of H₂O was added and the aqueous phase was extracted with CH₂Cl₂. The organic phases were combined and the solvent was evaporated. Compound II.1 was isolated from the reaction mixture via silica chromatography.

### Example 4: Production of 7-bromo-N-(cyclopropylmethyl)-1-oxo-indane-4-carboxamide (II.2)

Compound III.1 was cyclized to compound V.1 as described in Example 3. Compound V.1 was then amidated by addition of 172 mg cyclopropylmethanamine to the mixture to yield compound II.2.

### Example 5: Production of 7-bromo-1-oxo-indane-4-carboxylic acid (II.3)

Compound III.1 was cyclized to compound V.1 as described in Example 3. Compound V.1 was then treated with water to yield compound II.3.

### Example 6: Production of methyl 7-bromoindane-4-carboxylate (I.1)

A mixture of HgCl₂ (28 mg), H₂O (3 mL), concentrated aqueous HCl (0.025 mL) and Zn powder (370 mg) was produced. The mixture was stirred for 5 minutes at about 20-25° C., the supernatant was decanted. A premix of 3 mL of H₂O and 9 mL of concentrated aqueous HCl was added to the mixture, resulting in the production of gas. Compound II.1 (100 mg) from Example 3 was added to the mixture and stirred for 1.5 h under reflux. Workup was achieved by addition of brine and extraction with ethyl acetate. The organic phases were combined and the solvent was evaporated. Compound 1.1 was isolated by silica chromatography with a purity above 90% (determined by ¹H-NMR).

### Example 7: Production of Compound 1.1 from Compound II.1 by Reduction with NaBH₄, Followed by Dehydroxylation

A mixture of compound II.1 (180 mg) from Example 3 with CH₃OH (10 mL) was produced and cooled to 0° C. NaBH₄ (28 mg) was added to the mixture and stirred for 40 minutes at about 0° C., followed by 40 minutes at 20-25° C. Aqueous HCl (1 M) was added to a final pH of 7.0 and the solvent was evaporated. Methyl 7-bromo-1-hydroxy-indane-4-carboxylate (IIa.1) was produced at high purity (95%, determined by ¹H-NMR) with a yield of 89%.

A mixture of compound IIa (65 mg) with CH₂Cl₂ and trifluoroacetic acid (160 mg) was produced.

The mixture was stirred at about 25° C. for 70 h. Methyl 7-bromo-1-(2,2,2-trifluoroacetyl)oxy-indane-4-carboxylate was isolated by silica chromatography.

A mixture of methyl 7-bromo-1-(2,2,2-trifluoroacetyl)oxy-indane-4-carboxylate with 3 mL of trifluoroacetic acid and triethyl silane (67 mg) was produced. The mixture was stirred at 20-25° C. for 64 h, upon which the solvent was evaporated. Compound 1.1 was produced in pure form with a yield of 92% (determined by ¹H-NMR).

### Example 8: Characterization of Compound V.1 by NMR Spectroscopy

Compound V.1 was synthesized as described in Example 3. Before quenching, the reaction mixture was complemented with perdeuterated DMSO. The sample was measured by ¹H-NMR and ¹H-decoupled ¹³C-NMR at 500 MHz.

The following peaks were detected in the

a)¹³C-spectrum [ppm]: 27.58, 38.64, 162.27, 166.55, 223.00.

b)¹H-spectrum [ppm]: 3.02, 3.14, 7.49 (d), 8.07 (d).

The sample was also measured by ¹H-Correlation Spectroscopy (COSY) ¹H,¹³C-heteronuclear single quantum coherence (HSQC), and ¹H,¹H-heteronuclear multiple bond correlation (HMBC), resulting in the following peaks and their structural correlation:

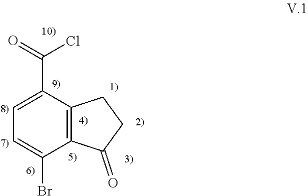 [SMILES: O=C(Cl)c1ccc(Br)c2c1CCC2=O]

Assignment: 1) (3.14/27.58); 2) (3.02/38.64); 3) (223.00), 4) (162); 5) (139.6), 6) (121.0); 7) (7.49/131.6); 8) (8.07/134.0); 9) (130.6), 10) (167.0).

### Example 9: Production of methyl 7-acetylindane-4-carboxylate (VI.1)

A mixture of methyl 7-bromoindane-4-carboxylate (compound I.1, 46 mg), ethylene glycol vinyl ether (79.5 mg), Pd(OAc)₂ (1 mg), 1,3-bis(diphenylphosphino)propane (3.7 mg), K₂CO₃ (74.5 mg) and 1 mL of water was stirred at 90° C. for 80 minutes under reflux. The mixture was cooled to about 25° C. and concentrated aqueous HCl (0.3 mL) was added. The resulting mixture was stirred at about 25° C. for one hour. Workup was achieved by addition of K₂CO₃ (10 wt % aqueous solution) and extraction by ethyl acetate. Then the organic phase was dried and concentrated. Compound VI.1 was isolated from the extract by silica column chromatography.

### Example 10: Production of methyl 7-[(Z)-3-(3,5-dichloro-4-fluoro-phenyl)-4,4,4-trifluoro-but-2-enoyl]indane-4-carboxylate (XIX.1)

A solution of methyl 7-acetylindane-4-carboxylate (compound VI.1, 50.00 g) in 1 liter 1,2-dichloroethane (DCE) was added to a mixture of 1-(3,5-dichloro-4-fluoro-phenyl)-2,2,2-trifluoro-ethanone (119.59 g), K₂CO₃ (38.00 g), and triethylamine (27.82 g). Then the mixture was stirred at about 120° C. under nitrogen for 12 h. Water (500 mL) was added to the mixture, which was in turn extracted with dichloromethane (DCM). Then the organic phase was dried and concentrated. Compound IX.1 was isolated by from the residue by silica column chromatography.

### Example 11: Production of methyl 7-[5-(3,5-dichloro-4-fluoro-phenyl)-5-(trifluoromethyl)-4H-isoxazol-3-yl]indane-4-carboxylate (XIV.1)

A mixture of a solution of compound XIX.1 of example 10 (100.00 g) in two liters DCE, NH₂OH/HCl (30.13 g), and tetra-n-butylammonium bromide (10.48 g) was produced. A solution of NaOH (34.69 g) in 400 mL water was admixed dropwise at about 25° C. Then the mixture was stirred at about 25° C. for 12 h. Water (200 mL) was added to the mixture, which was in turn extracted with DCM. The organic phase was washed with water, dried, filtered and concentrated by evaporation of the solvent. Compound XIV.1 was isolated from the residue by silica column chromatography with a yield of 96.8%.

### Example 12: Production of 7-[5-(3,5-dichloro-4-fluoro-phenyl)-5-(trifluoromethyl)-4H-isoxazol-3-yl]indane-4-carboxylic acid (XIV.2)

A solution of compound XIV.1 of example 11 (110.00 g) in THF/dioxane (500 mL/500 mL), and a solution of LiOH.H₂O (48.46 g) in 150 mL water was mixed at about 25° C. Then the mixture was stirred at about 80° C. for 18 h, cooled to about 25° C., and concentrated under reduced pressure at about 50° C. Water (400 mL) was added to the mixture, which was subsequently extracted with petroleum ether (400 mL). The pH of the aqueous phase was then adjusted to about 2.0 with aqueous HCl. The aqueous phase was subsequently extracted with ethyl acetate. The ethyl acetate extract was dried, and concentrated in vacuo. The residue was washed with hexane (600 mL), resulting in compound XIV.2 with a yield of 93.7% as a white solid.

### Example 13: Production of 7-[5-(3,5-dichloro-4-fluoro-phenyl)-5-(trifluoromethyl)-4H-isoxazol-3-yl]-N-(pyrimidin-2-ylmethyl)indane-4-carboxamide (XIV.3)

A solution of compound XIV.2 of example 12 (230 mg), the HCl salt of pyrimidin-2-ylmethanamine (65 mg), and PyBrop (279 mg), in DCM (15 mL) was produced. Di-isopropyl ethyl amine (258 mg) was admixed at 20 to 25° C. The mixture was stirred overnight under nitrogen. The reaction was extracted with water, and the organic phase was concentrated in vacuo. Compound XIV.3 was isolated from the residue via silica column chromatography.

### Example 14: Production of methyl 7-[(Z)-3-(3,5-dichlorophenyl)-4,4,4-trifluoro-but-2-enoyl]indane-4-carboxylate (XIX.2)

A mixture of 1-[7-(1-methoxyvinyl)indan-4-yl]ethanone (27 g), 1-(3,5-dichlorophenyl)-2,2,2-trifluoro-ethanone (60 g), K₂CO₃ (22 g), triethylamine (16 g) and dichloroethane (600 mLl) was produced. The mixture was stirred at 110° C. under nitrogen for 12 h. Water (500 mL) was added to the mixture. The mixture was subsequently extracted with DCM. The organic phase was dried over N₂SO₄ and concentrated in vacuo. Compound XIX.2 was isolated from the residue via silica column chromatography with a yield of 87.8%.

### Example 15: Production of methyl 7-[3-(3,5-dichlorophenyl)-4,4,4-trifluoro-3-(nitromethyl)butanoyl]indane-4-carboxylate

A mixture of compound XIX.2 of example 14 (40 g), MeCN (400 mL), 1,8-diazabicyclo[5.4.0]un-dec-7-ene (41 g) and nitromethane (27 g) was produced. The mixture was stirred for 40 min at 20° C. and subsequently adjusted to pH 5-6 with aqueous HCl. The mixture was then extracted with ethyl acetate. The organic phase was concentrated in vacuo. Methyl 7-[3-(3,5-dichlorophenyl)-4,4,4-trifluoro-3-(nitromethyl)butanoyl]indane-4-carboxylate was isolated from the residue via silica column chromatography with a yield of 80%.

### Example 16: Production of Methyl 7-[3-(3,5-dichlorophenyl)-3-(trifluoromethyl)-2,4-dihydropyrrol-5-yl]indane-4-carboxylate (XIV.4)

Iron powder (10.4 g) was added at about 25° C. to a mixture of methyl 7-[3-(3,5-dichlorophenyl)-4,4,4-trifluoro-3-(nitromethyl)butanoyl]indane-4-carboxylate of example 15, CH₃OH (300 mL), and CH₃COOH (300 mL). The mixture was stirred at 80° C. for 12 h. The mixture was concentrated in vacuo and subsequently poured into a saturated solution of aqueous NaHCO₃. The resulting mixture was extracted with ethyl acetate. The organic phase was concentrated in vacuo. Compound XIV.4 was isolated from the residue via silica column chromatography.

### Example 17: Production of 7-[3-(3,5-dichlorophenyl)-3-(trifluoromethyl)-2,4-dihydropyrrol-5-yl]indane-4-carboxylic acid (XIV.5)

A mixture of compound XIV.4 of example 16 (19 g), THF (200 mL) and CH₃OH (100 mL) was produced. A solution of LiOH.H₂O (8.4 g) in water (100 mL) was added to the mixture at 25° C., which was subsequently stirred at 20° C. for 12 h. The pH of the mixture was then adjusted to pH 9 with aqueous HCl. The mixture was subsequently reduced in vacuo, upon which the pH was again adjusted to pH 5 with aqueous HCl. The resulting mixture was then extracted with ethyl acetate. The organic phase was concentrated in vacuo. Compound XIV.5 was isolated from the residue via silica column chromatography.

### Example 18: Production of 7-[3-(3,5-dichlorophenyl)-3-(trifluoromethyl)-2,4-dihydropyrrol-5-yl]-N-(pyrimidin-2-ylmethyl)indane-4-carboxamide (XIV.6)

A solution of compound XIV.5 (1.0 eq), pyrimidin-2-ylmethanammoniumchloride (1.2 eq), PyBrOP (1.2 eq) in DCM (10 mL) was produced. Diisopropylethylamine (3-4 eq) was added to the mixture at 20 to 25° C. The mixture was stirred under nitrogen for several hours. The mixture was extracted with water, and the organic phase was reduced in vacuo. Compound XIV.6 was isolated from the residue via silica column chromatography.

### Example 19: Production of 7-[3-(3,5-dichloro-4-fluoro-phenyl)-3-(trifluoromethyl)-2,4-dihydropyrrol-5-yl]-N-(pyrimidin-2-ylmethyl)indane-4-carboxamide (XIV.7)

Compound 7-[3-(3,5-dichloro-4-fluoro-phenyl)-3-(trifluoromethyl)-2,4-dihydropyrrol-5-yl]indane-4-carboxylic acid was produced in analogy to examples 14 to 17. A solution of 7-[3-(3,5-dichloro-4-fluoro-phenyl)-3-(trifluoromethyl)-2,4-dihydropyrrol-5-yl]indane-4-carboxylic acid (2.5 g), pyrimidin-2-ylmethanammoniumchloride (0.95 g), PyBrOP (3.04 g) in DCM (100 mL) was produced. Diisopropylethylamine (2.25 g) was added to the mixture at 20 to 25° C. The mixture was stirred under nitrogen for several hours. The mixture was extracted with water, and the organic phase was reduced in vacuo. Compound XIV.7 was isolated from the residue via silica column chromatography.

### Example 20: Production of 5-(3,5-dichlorophenyl)-3-hydroxy-3-(7-methoxycarbonylindan-4-yl)-5-(trifluoromethyl)tetrahydrothiophene-2-carboxylic acid

2-sulfanylacetic acid (2.2 g) and triethylamine (2.4 g) were added to a mixture of compound XIX.2 of example 14 (2.2 g) and THF (35 mL). The resulting mixture was stirred at 20° C. for 16 h and reduced in vacuo. Subsequently, the pH was adjusted to pH 2 with aqueous HCl. The mixture was extracted with methyl-tert-butyl-ether (50 mL). Subsequently, the pH of the aqueous phase was adjusted to pH 8 with a saturated solution of NaHCO₃. The aqueous phase was then extracted with ethyl acetate and reduced in vacuo to dryness, thereby yielding 3-(7-carboxyindan-4-yl)-5-(3,5-dichlorophenyl)-3-hydroxy-5-(trifluoromethyl)tetrahydrothiophene-2-carboxylic acid.

### Example 21: Production of methyl 7-[2-(3,5-dichlorophenyl)-2-(trifluoromethyl)-3H-thiophen-4-yl]indane-4-carboxylate (XIV.8)

Mesyl chloride (3 g) was added to a mixture of 5-(3,5-dichlorophenyl)-3-hydroxy-3-(7-methoxycarbonylindan-4-yl)-5-(trifluoromethyl)tetrahydrothiophene-2-carboxylic acid of example 20 (6.2 g) and pyridine (60 mL) at 0° C. The mixture was subsequently stirred at 20° C. for 16 h, then poured into H₂O (100 mL) and extracted with ethyl acetate. The organic phase was washed with brine, dried, and reduced in vacuo. DMF (20 mL) was added to the residue, and the resulting mixture was stirred at 120° C. for 1 h. The mixture was reduced in vacuo and then poured into H₂O (100 mL) and extracted with methyl tert-butyl ether. The organic phase was reduced in vacuo. Compound XIV.8 was isolated from the residue via silica column chromatography.

### Example 22: Production of 7-[2-(3,5-dichlorophenyl)-2-(trifluoromethyl)-3H-thiophen-4-yl]indane-4-carboxylic acid (XIV.9)

A mixture of compound XIV.8 of example 21 (1.1 g), and THF (15 mL) was produced. A solution of LiOH.H₂O (0.3 g) in water (1 mL) was added to the mixture, which was subsequently stirred at 20° C. for 16 h. The pH of the mixture was then adjusted to pH 2 with aqueous HCl. The resulting mixture was then extracted with ethyl acetate. The organic phase was washed with brine, dried, and concentrated in vacuo. Compound XIV.9 was isolated from the residue via silica column chromatography.

### Example 23: Production of Dihydrothiophene Compounds XIV.10 to XIV.13

A solution comprising compound XIV.9 of example 22 (1.0 eq), PyBrOP (1.2 eq), DCM (10 mL), and an ammonium chloride compound selected from pyrimidin-2-ylmethanammonium chloride, 2-pyridylmethanammonium chloride, 2-ammonium-N-(2,2,2-trifluoroethyl)acetamide chloride, or 1,1-dioxothietan-3-ammonium chloride (1.2 eq) was produced. Diisopropylethylamine (3-4 eq) was added to the mixture at 20 to 25° C. The mixture was stirred under nitrogen for several hours. The mixture was extracted with water, and the organic phase was reduced in vacuo. Depending on the ammonium chloride compound used, a compound XIV.10 to XIV.13 was isolated from the residue via silica column chromatography.

### Example 24: Production of Dihydrothiophene Compounds XIV.15 to XIV.20

7-[2-(3,5-dichloro-4-fluoro-phenyl)-2-(trifluoromethyl)-3H-thiophen-4-yl]indane-4-carboxylic acid (XIV.14) was produced in analogy to compound XIV.9 in examples 20 to 22. A solution comprising compound XIV.14 (1.0 eq), PyBrOP (1.2 eq), DCM (10 mL), and an ammonium chloride compound selected from 1,1-dioxothietan-3-ammonium chloride, (4R)-4-ammonium-2-ethyl-isoxazolidin-3-one chloride, pyrimidin-2-ylmethanammonium chloride, 2-ammonium-N-(2,2,2-trifluoroethyl)acetamide chloride, thietan-3-ammonium chloride, and 2-methylsulfanylethanammonium chloride (1.2 eq) was produced. Diisopropylethylamine (3-4 eq) was added to the mixture at 20 to 25° C. The mixture was stirred under nitrogen for several hours. The mixture was extracted with water, and the organic phase was reduced in vacuo. Depending on the ammonium chloride compound used, a compound XIV.15 to XIV.20 was isolated from the residue via silica column chromatography.

### Example 25: Production of [7-[5-(3,5-dichloro-4-fluoro-phenyl)-5-(trifluoromethyl)-4H-isoxazol-3-yl]indan-4-yl]methanol

LiBH₄ (0.44 g) was added to a mixture of compound XIV.1 of example 11 (2 g), and THF (50 mL) at 25° C. The mixture was stirred at 70° C. for 15 h and subsequently diluted with a saturated aqueous solution of NH₄Cl. The mixture was extracted with ethyl acetate. The organic phases were dried, reduced in vacuo. [7-[5-(3,5-dichloro-4-fluoro-phenyl)-5-(trifluoromethyl)-4H-isoxazol-3-yl]indan-4-yl]methanol was isolated from the residue by silica column chromatography.

### Example 26: Production of 3-[7-(chloromethyl)indan-4-yl]-5-(3,5-dichloro-4-fluoro-phenyl)-5-(trifluoro-methyl)-4H-isoxazole

To a solution of [7-[5-(3,5-dichloro-4-fluoro-phenyl)-5-(trifluoromethyl)-4H-isoxazol-3-yl]indan-4-yl]methanol (2.0 g) in DCM (60 mL) was admixed triethylamine (3 mL) and mesylchloride (1 g) at 0° C. The mixture was stirred at 25° C. for 10 h. The mixture was subsequently diluted with a saturated aqueous solution of NH₄Cl. The mixture was extracted with ethyl acetate. The organic phases were dried, reduced in vacuo. [7-[5-(3,5-dichloro-4-fluoro-phenyl)-5-(trifluoromethyl)-4H-isoxazol-3-yl]indan-4-yl]methanol was isolated from the residue by silica column chromatography with a yield of 94%.

### Example 27: Production of [7-[5-(3,5-dichloro-4-fluoro-phenyl)-5-(trifluoromethyl)-4H-isoxazol-3-yl]indan-4-yl]methanamine

A mixture of 3-[7-(chloromethyl)indan-4-yl]-5-(3,5-dichloro-4-fluoro-phenyl)-5-(trifluoro-methyl)-4H-isoxazole of example 26 (3.8 g), NaN₃ (0.95 g), and DMF (50 mL) was stirred at 25° C. under nitrogen for 13 h. The mixture was diluted with water (100 mL) and extracted with methyl tert-butyl ether. The organic phases were dried and reduced in vacuo. The residue was dissolved in THF (80 mL) and H₂O (80 mL). Triphenylphosphine (2 g) was added, and the mixture was stirred at 85° C. under nitrogen for 2 h. The mixture was extracted with ethyl acetate and the organic phases were dried and concentrated. [7-[5-(3,5-dichloro-4-fluoro-phenyl)-5-(trifluoromethyl)-4H-isoxazol-3-yl]indan-4-yl]methanamine was isolated from the residue by silica column chromatography.

### Example 27: Production of Inverted Amides XV.1 to XV.6

A mixture of [7-[5-(3,5-dichloro-4-fluoro-phenyl)-5-(trifluoromethyl)-4H-isoxazol-3-yl]indan-4-yl]methanamine of example 26 (1.0 eq) in THF (10 mL) and TEA (3.0 eq.) was stirred at 20 to 25° C. An acid chloride (1.2 eq.) selected from 2-methylsulfonylacetyl chloride, propanoyl chloride, 3,3,3-trifluoropropanoyl chloride, acetyl chloride, cyclopropanecarbonyl chloride was added dropwise. The reaction mixture was stirred at 20 to 25° C., followed by dilution with H₂O and extraction with ethyl acetate. The organic phases were dried and reduced in vacuo. Depending on the acid chloride compound used, a compound XV.1 to XIV.6 was isolated from the residue via silica column chromatography.

### Example 28: Production of Inverted Amides XV.7 to XV.8

Compound [7-[5-(3,5-dichlorophenyl)-5-(trifluoromethyl)-4H-isoxazol-3-yl]indan-4-yl]methanamine was produced in analogy to [7-[5-(3,5-dichloro-4-fluoro-phenyl)-5-(trifluoromethyl)-4H-isoxazol-3-yl]indan-4-yl]methanamine in examples 25 to 27.

A mixture of [7-[5-(3,5-dichlorophenyl)-5-(trifluoromethyl)-4H-isoxazol-3-yl]indan-4-yl]methanamine (1.0 eq) in THF (10 mL) and TEA (3.0 eq.) was stirred at 20 to 25° C.

An acid chloride (1.2 eq.) selected from 2-methylsulfonylacetyl chloride, and propanoyl chloride, was added dropwise. The reaction mixture was stirred at 20 to 25° C., followed by dilution with H₂O and extraction with ethyl acetate. The organic phases were dried and reduced in vacuo. Depending on the acid chloride compound used, a compound XV.7 or XIV.8 was isolated from the residue via silica column chromatography.

### Example 29: Production of Further Compounds XIV-A and XV-A

Tables 3, 4, 5 and 6 list further compounds XIV-A and XV-A from the classes of isoxazazoles, pyrrolines, dihydrothophenes, and dihydrofuranes. These were produced in analogy to examples 1 to 28, or in accordance to the general description of the reactions above from compounds I.1 and VI.1 with the respective amines, acid chlorides, 2,2,2-trifluoroacetophenone, and 4-halogen-2-phenyl-2-(trifluoromethyl)-3H-furan derivatives.

TABLE 2
Characterization of compounds by NMR spectroscopy

| Compound | Solvent/frequency | Chemical shift ¹H-NMR (ppm) | Chemical shift ¹³C-NMR (ppm) |
|---|---|---|---|
| I.1 | DMSO-d6/500 | 2.04 (m, 2H), 2.9 (t, 2H), 3.3 (t, 2H), 3.8 (s, | 23.3, 33.9, 34.77, 52.04, 124.36, |
| | MHz | 3H), 7.52 (d, 1H), 7.64 (d, 1H) | 125.36, 129.65, 129.68, 145.68, 147.9, |
| | | | 166.12. |
| II.1 | DMSO-d6/500 | 2.71 (m, 2H), 3.34 (m, 2H), 3.9 (s, 3H), | 25.82, 36.44, 52.21, 123.48, 126.29, |
| | MHz | 7.78 (d, 1H), 8.06 (d, 1H). | 132.66, 134.58, 136.14, 159.27, 165.06, |
| | | | 203.05. |
| II.2 | CDCl₃/500 | 0.31 (q, 4H), 1.06 (m, 1H), 2.77 (m, 2H), | 3.60 (2C), 10.70, 24.94, 36.94, 44.90, |
| | MHz | 3.3 (m, 4H), 6.22 (t, 1H), 7.59 (d, 1H), 7.60 | 122.21, 132.42, 132.62, 132.75, 134.97, |
| | | (d, 1H) | 156.89, 166.38, 203.68 |
| II.3 | DMSO-d6/500 | 2.69(m, 2H), 3.35 (m, 2H), 7.74 (d, 1H), | 26.09, 36.63, 123.14, 128.20, 132.66, |
| | MHz | 8.04 (d, 1H), 13.4 (s, broad, 1H) | 134.59, 136.61, 159.56, 166.42, 203.36 |
| III.1 | DMSO-d6/500 | 2.51 (t, 2H), 3.12 (t, 2H), 7.5 (s, 1H), 7.6 | 28.59, 34.84, 125.26, 129.24, 129.42, |
| | MHz | (d, 1H), 7.7 (d, 1H), 12.5 (s, broad, 2H) | 132.27, 133.33, 144.39, 167.72, 173.46 |
| V.1 | DMSO-d6/500 | 3.02 (m, 2H), 3.14 (m, 2H), 7.49 (d, 1H), | 27.58, 38.64, 121.0, 130.6, 131.6, |
| | MHz | 8.07 (d, 1H) | 134.0, 139.6, 162.27, 167.0, 223.00 |
| VI.1 | CDCl₃/500 | 2.09 (m, J = 7.7 Hz, 2 H) 2.62 (s, 3 H) 3.26 | 24.85, 28.74, 33.36, 33.64, 52.07, |
| | MHz | (m, 4 H) 3.93 (s, 3 H) 7.7 (d, J = 8.1 Hz, 1 | 127.11, 128.15, 129.47, 136.65, 146.96, |
| | | H) 7.90 (d, J = 8.1 Hz, 1 H) | 148.77, 166.95, 199.85 |
| XIV.1 | CDCl₃/400 | 7.809-7.789 (d, J = 8 Hz, 1 H) 7.527-7.513 | — |
| | MHz | (d, J = 5.6 Hz, 1 H) 7.187-7.141 (t, J = 9.2 | |
| | | Hz, 1H) 4.099-4.056 (d, J = 17.2 Hz, 1H) | |
| | | 3.84 (s, 3H), 3.704-3.662 (d, J = 16.8 Hz), | |
| | | 3.264-3.226 (m, 2H), 3.137-3.120 (m, 2 H) | |
| | | 2.068-2.030 (m, 2 H). | |
| XIV.2 | DMSO-d6/500 | 13.10 (s, 1H, very broad), 7.84 (d, 2H, | 167.60, 157.84, 153.74, 146.97, 144.57, |
| | MHz | J = 6 Hz), 7.81 (d, 1H, J = 8 Hz), 7.50 (d, 1H, | 132.95, 129.69, 128.05, 128.05, 128.05, |
| | | J = 8 Hz), 4.41 (d, 1H, J = 18.3 Hz), 4.33 (d, | 126.88, 126.34, 123.68, 121.59, 121.59, |
| | | 1H, J = 18.3 Hz), 3.23 (t, 2H, J = 7.7 Hz), 3.09 | 85.63, 43.95, 34.05, 33.25, 24.01 |
| | | (m 2H), 2.03 (m, 2H) | |
| XIV.3 | DMSO-d6/500 | 8.75 (d, 2H, J = 4.9 Hz), 7.65 (d, 1H, | 167.74, 165.55, 157.29, 157.29, 156.32, |
| | MHz | J = 8 Hz), 7.61 (d, 2H, J = 6 Hz), 7.45 (t, 1H, | 154.76, 145.91, 144.94, 133.55, 133.08, |
| | | J = 4.5 Hz), 7.27 (m, 2H), 4.91 (d, 2H, | 127.57, 127.57, 126.32, 125.91, 125.79, |
| | | J = 4.5 Hz), 4.17 (d, 1H, J = 17 Hz), 3.79 (d, | 123.77, 123.06, 123.06, 119.73, 86.14, |
| | | 1H, J = 17 Hz), 3.28 (t, 2H, J = 7.7 Hz), 3.22 | 45.68, 45.29, 34.93, 33.03, 25.00 |
| | | (q, 2H, J = 7.7 Hz), 2.15 (m, 2H) | |
| XIV.4 | CDCl₃/400 | 7.82 (d, J = 8.38 Hz, 1 H), 7.39 (d, J = 7.94 | |
| | MHz | Hz, 1 H), 7.31 (t, J = 1.54 Hz, 1 H), 7.19 (d, | |
| | | J = 1.32 Hz, 2 H), 4.87 (dd, J = 17.20, 1.32 | |
| | | Hz, 1 H), 4.40 (d, J = 17.20 Hz, 1H), 3.85 | |
| | | (s, 3 H), 3.73 (dd, J = 17.42, | |
| | | 1.54 Hz, 1 H), 3.42 (d, J = 17.20 Hz, 1H), | |
| | | 3.12-3.29 (m, 4 H), 2.03 (t, J = 7.28 Hz, 2H) | |
| XIV.5 | DMSO-d6/500 | 13.0 (s, 1H, broad), 7.82 (d, 1H, J = 8 Hz), | 171.09, 167.52, 147.14, 145.16, 141.08, |
| | MHz | 7.69 (m, 2H), 7.60 (d, 2H, J = 1.4 Hz), 4.91 | 134.15, 134.15, 132.62, 128.33, 128.1, |
| | | (d, 1H, J = 17.3 Hz), 4.45 (d, 1H, J = 17.3 Hz), | 127.9, 127.9, 127.9, 127.36, 127.18, |
| | | 3.88 (d, 1H, J = 17.9 Hz), 3.77 (d, 1H, | 67.22, 54.81, 43.78, 33.62, 33.12, 24.21 |
| | | J = 17.9 Hz), 3.21 (m, 2H), 2.01 (m, 2H) | |
| XIV.6 | DMSO-d6/500 | 8.75 (d, 2H, J = 5 Hz), 7.66 (d, 1H, J = 8 Hz), | 135.29, 135.29, 133.3, 131.57, 128.57, |
| | MHz | 7.51 (d, 1H, J = 8 Hz), 7.45 (t, 1H, J = 4.3 Hz), | 127.15, 127.13, 127.13, 126.88, 125.52, |
| | | 7.38 (t, 1H, J = 1.7 Hz), 7.27 (m, 3H), 4.94 | 119.63, 68.43, 55.12, 45.61, 45.05, |
| | | (d, 1H, J = 17.3 Hz), 4.92 (d, 2H, J = 4.3 Hz), | 34.24, 32.78, 25.23 |
| | | 4.47 (d, 1H, J = 17.3 Hz), 3.81 (d, 1H, | |
| | | J = 17.3 Hz), 3.51 (d, 1H, J = 17.3 Hz), 3.26 | |
| | | (m, 4H), 2.13 (m, 2H) | |
| XIV.8 | CDCl₃/400 | 7.84 (d, J = 8.03 Hz, 1H) 7.37-7.46 (m, | |
| | MHz | 3H) 7.11 (d, J = 8.28 Hz, 1H) 6.45 (s, 1H) | |
| | | 3.87-3.94 (m, 4H) 3.70-3.77 (m, 1H) | |
| | | 3.29-3.36 (m, 2H) 2.96-3.02 (m, 2H) | |
| | | 2.08-2.16 (m, 2H) | |
| XIV.9 | CDCl₃/400 | 7.94 (d, J = 8.38 Hz, 1H) 7.38-7.45 (m, 3 | |
| | MHz | H) 7.15 (d, J = 7.94 Hz, 1H) 3.87-3.97 (m, | |
| | | 1H), 3.68-3.80 (m, 2H) 3.32-3.42 (m, | |
| | | 2H) 3.01 (td, J = 7.39, 2.43 Hz, 2H) 2.06- | |
| | | 2.19 (m, 3H) | |
| XIV.56 | DMSO-d6/500 | 7.6 (d, 2H, J = 6 Hz), 7.44 (d, 1H, J = 8 Hz), | 168.17, 156.11, 154.73, 146.15, 145.47, |
| | MHz | 7.2 (d, 1H, J = 8 Hz), 7.11 (d, 1H, J = 6.7 Hz), | 132.84, 131.68, 127.5, 127.5, 126.22, |
| | | 4.86 (m, 1H), 4.59 (m, 2H), 4.11 (m, 3H), | 126.5, 125.33, 123.66, 123.02, 123.02, |
| | | 3.77 (d, 1H, J = 17.3 Hz), 3.15 (m, 4H), 2.1 | 86.24, 71.48, 71.48, 45.08, 34.74, |
| | | (m, 2H) | 32.83, 32.25, 24.79 |
| XIX.1 | DMSO-d6/500 | 7.86 (d, 1H, J = 8 Hz), 7.49 (d, 1H, J = 8 Hz), | 192.26, 166.51, 154.67, 149.11, 147.63, |
| | MHz | 7.32 (q, 1H, J = 1.1 Hz), 7.19 (d, 2H, | 135.88, 135.23, 133.35, 130.46, 129.64, |
| | | J = 6 Hz), 3.92 (s, 3H,), 3.24 (t, 2H, | 129.64, 128.25, 127.63, 127.16, 122.63, |
| | | J = 7.6 Hz), 3.1 (t, 2H, J = 7.6 Hz), 2.07 (quin- | 122.63, 122.18, 52.22, 33.33, 32.25, |
| | | tet, 2H, J = 7.6 Hz) | 24.86 |
| XIX.2 | CDCl₃/400 | 7.81 (d, 1H, J = 8.38 Hz), 7.44 (d, 1H, | |
| | MHz | J = 7.94 Hz), 7.25 (d, 2H, 7.06 Hz), 7.07 (d, | |
| | | 2H, 0.88 Hz), 3.89 (s, 3H), 3.21 (t, 2H, | |
| | | J = 7.72 Hz), 3.06 (t, 2H, J = 7.5 Hz), 2.05 (d, | |
| | | 2H, J = 7.5 Hz) | |

TABLE 3
Characterization of dihydrothiophene compounds by HPLC-MS

| | | | Mass over |
|---|---|---|---|
| | HPLC | Retention | charge |
| Compound | set-up | time | (m/z) |
| 7-[2-(3,5-dichlorophenyl)-2-(trifluoromethyl)-3H-thiophen-4- | A | 1.440 | 550.0 |
| yl]-N-(pyrimidin-2-ylmethyl)indane-4-carboxamide (XIV.10) | | | |
| 7-[2-(3,5-dichlorophenyl)-2-(trifluoromethyl)-3H-thiophen-4- | A | 1.267 | 549.0 |
| yl]-N-(2-pyridylmethyl)indane-4-carboxamide (XIV.11) | | | |
| 7-[2-(3,5-dichlorophenyl)-2-(trifluoromethyl)-3H-thiophen-4- | A | 1.432 | 596.9 |
| yl]-N-[2-oxo-2-(2,2,2-trifluoroethylamino)ethyl]indane- | | | |
| 4-carboxamide (XIV.12) | | | |
| 7-[2-(3,5-dichlorophenyl)-2-(trifluoromethyl)-3H-thiophen-4- | A | 1.416 | 561.9 |
| yl]-N-(1,1-dioxothietan-3-yl)indane-4-carboxamide (XIV.13) | | | |
| 7-[2-(3,5-dichloro-4-fluoro-phenyl)-2-(trifluoromethyl)-3H- | B | 1.428 | 579.6 |
| thiophen-4-yl]-N-(1,1-dioxothietan-3-yl)indane-4-carboxamide | | | |
| (XIV.15) | | | |
| 7-[2-(3,5-dichloro-4-fluoro-phenyl)-2-(trifluoromethyl)-3H- | A | 1.464 | 590.4 |
| thiophen-4-yl]-N-[(4R)-2-ethyl-3-oxo-isoxazolidin-4- | | | |
| yl]indane-4-carboxamide (XIV.16) | | | |
| 7-[2-(3,5-dichloro-4-fluoro-phenyl)-2-(trifluoromethyl)-3H- | A | 1.429 | 568.0 |
| thiophen-4-yl]-N-(pyrimidin-2-ylmethyl)indane-4- | | | |
| carboxamide (XIV.17) | | | |
| 7-[2-(3,5-dichloro-4-fluoro-phenyl)-2-(trifluoromethyl)-3H- | A | 1.451 | 614.6 |
| thiophen-4-yl]-N-[2-oxo-2-(2,2,2- | | | |
| trifluoroethylamino)ethyl]indane-4-carboxamide (XIV.18) | | | |
| 7-[2-(3,5-dichloro-4-fluoro-phenyl)-2-(trifluoromethyl)-3H- | B | 1.467 | 548.0 |
| thiophen-4-yl]-N-(thietan-3-yl)indane-4-carboxamide (XIV.19) | | | |
| 7-[2-(3,5-dichloro-4-fluoro-phenyl)-2-(trifluoromethyl)-3H- | A | 1.462 | 550.0 |
| thiophen-4-yl]-N-(2-methylsulfanylethyl)indane-4-carboxamide | | | |
| (XIV.20) | | | |

TABLE 4
Characterization of inverted amide compounds by HPLC-MS

| | | | Mass over |
|---|---|---|---|
| | HPLC | Retention | charge |
| Compound | set-up | time | (m/z) |
| N-[[7-[5-(3,5-dichloro-4-fluoro-phenyl)-5-(trifluoromethyl)-4H- | A | 1.367 | 567.0 |
| isoxazol-3-yl]indan-4-yl]methyl]-2-methylsulfonyl-acetamide (XV.1) | | | |
| N-[[7-[5-(3,5-dichloro-4-fluoro-phenyl)-5-(trifluoromethyl)-4H- | A | 1.421 | 503.0 |
| isoxazol-3-yl]indan-4-yl]methyl]propanamide (XV.2) | | | |
| N-[[7-[5-(3,5-dichloro-4-fluoro-phenyl)-5-(trifluoromethyl)-4H- | A | 1.455 | 557.0 |
| isoxazol-3-yl]indan-4-yl]methyl]-3,3,3-trifluoro-propanamide (XV.3) | | | |
| N-[[7-[5-(3,5-dichloro-4-fluoro-phenyl)-5-(trifluoromethyl)-4H- | A | 1.384 | 488.9 |
| isoxazol-3-yl]indan-4-yl]methyl]acetamide (XV.4) | | | |
| N-[[7-[5-(3,5-dichloro-4-fluoro-phenyl)-5-(trifluoromethyl)-4H- | A | 1.423 | 514.9 |
| isoxazol-3-yl]indan-4-yl]methyl]cyclopropanecarboxamide (XV.5) | | | |
| N-[[7-[5-(3,5-dichloro-4-fluoro-phenyl)-5-(trifluoromethyl)-4H- | A | 1.421 | 503.0 |
| isoxazol-3-yl]indan-4-yl]methyl]propanamide (XV.6) | | | |
| N-[[7-[3-(3,5-dichlorophenyl)-3-(trifluoromethyl)-2,4-dihydropyrrol- | A | 1.270 | 483.1 |
| 5-yl]indan-4-yl]methyl]propanamide (XV.7) | | | |
| N-[[7-[3-(3,5-dichlorophenyl)-3-(trifluoromethyl)-2,4-dihydropyrrol- | C | 1.222 | 547.1 |
| 5-yl]indan-4-yl]methyl]-2-methylsulfonyl-acetamide (XV.8) | | | |

TABLE 5
Characterization of amide compounds by HPLC-MS

| | | | Mass over |
|---|---|---|---|
| | HPLC | Retention | charge |
| Compound | set-up | time | (m/z) |
| 7-[3-(3,5-dichloro-4-fluoro-phenyl)-3-(trifluoromethyl)-2,4- | A | 1.284 | 550.9 |
| dihydropyrrol-5-yl]-N-(pyrimidin-2-ylmethyl)indane-4- | | | |
| carboxamide (XIV.7) | | | |
| 7-[3-(3,5-dichloro-4-fluoro-phenyl)-3-(trifluoromethyl)-2,4- | A | 1.405 | 535.0 |
| dihydropyrrol-5-yl]-N-(2-methylsulfanylethyl)indane-4- | | | |
| carboxamide (XIV.21) | | | |
| 7-[3-(3,5-dichloro-4-fluoro-phenyl)-3-(trifluoromethyl)-2,4- | A | 1.284 | 550.9 |
| dihydropyrrol-5-yl]-N-(pyrimidin-2-ylmethyl)indane-4- | | | |
| carboxamide (XIV.22) | | | |
| 7-[3-(3,5-dichloro-4-fluoro-phenyl)-3-(trifluoromethyl)-2,4- | A | 1.527 | 544.8 |
| dihydropyrrol-5-yl]-N-(thietan-3-ylmethyl)indane-4-carboxamide | | | |
| (XIV.23) | | | |
| N-(cyclopropylmethyl)-7-[3-(3,5-dichloro-4-fluoro-phenyl)-3- | A | 1.393 | 514.6 |
| (trifluoromethyl)-2,4-dihydropyrrol-5-yl]indane-4-carboxamide | | | |
| (XIV.24) | | | |
| 7-[3-(3,5-dichloro-4-fluoro-phenyl)-3-(trifluoromethyl)-2,4- | A | 1.265 | 576.9 |
| dihydropyrrol-5-yl]-N-[(1,1-dioxothietan-3-yl)methyl]indane- | | | |
| 4-carboxamide (XIV.25) | | | |
| N-cyclopropyl-7-[3-(3,5-dichlorophenyl)-3-(trifluoromethyl)-2,4- | A | 1.347 | 481.0 |
| dihydropyrrol-5-yl]indane-4-carboxamide (XIV.26) | | | |
| 7-[3-(3,5-dichlorophenyl)-3-(trifluoromethyl)-2,4-dihydropyrrol- | A | 1.319 | 538.0 |
| 5-yl]-N-(thiazol-4-ylmethyl)indane-4-carboxamide (XIV.27) | | | |
| 7-[3-(3,5-dichlorophenyl)-3-(trifluoromethyl)-2,4-dihydropyrrol- | A | 1.357 | 594.0 |
| 5-yl]-N-[1-methyl-2-oxo-2-(2,2,2- | | | |
| trifluoroethylamino)ethyl]indane-4-carboxamide (XIV.28) | | | |
| 7-[3-(3,5-dichlorophenyl)-3-(trifluoromethyl)-2,4-dihydropyrrol- | A | 1.232 | 529.0 |
| 5-yl]-N-(1-oxothietan-3-yl)indane-4-carboxamide (XIV.29) | | | |
| 7-[3-(3,5-dichloro-4-fluoro-phenyl)-3-(trifluoromethyl)-2,4- | A | 1.468 | 515.8 |
| dihydropyrrol-5-yl]-N-[(E)-methoxyiminomethyl]indane-4- | | | |
| carboxamide (XIV.30) | | | |
| 1-[[7-[3-(3,5-dichlorophenyl)-3-(trifluoromethyl)-2,4- | A | 1.286 | 581.3 |
| dihydropyrrol-5-yl]indane-4-carbonyl]amino]-3-(2,2,2- | | | |
| trifluoroethyl)urea (XIV.31) | | | |
| 7-[3-(3,5-dichloro-4-fluoro-phenyl)-3-(trifluoromethyl)-2,4- | A | 1.271 | 565.3 |
| dihydropyrrol-5-yl]-N-(2-methylsulfonylethyl)indane-4- | | | |
| carboxamide (XIV.32) | | | |
| 7-[3-(3,5-dichloro-4-fluoro-phenyl)-3-(trifluoromethyl)-2,4- | A | 1.433 | 565.0 |
| dihydropyrrol-5-yl]-N-[[1- | | | |
| (difluoromethyl)cyclopropyl]methyl]indane-4-carboxamide (XIV.33) | | | |
| 7-[3-(3,5-dichloro-4-fluoro-phenyl)-3-(trifluoromethyl)-2,4- | A | 1.358 | 498.9 |
| dihydropyrrol-5-yl]-N-prop-2-ynyl-indane-4-carboxamide (XIV.34) | | | |
| 7-[3-(3,5-dichloro-4-fluoro-phenyl)-3-(trifluoromethyl)-2,4- | A | 1.411 | 549.3 |
| dihydropyrrol-5-yl]-N-[(2,2- | | | |
| difluorocyclopropyl)methyl]indane-4-carboxamide (XIV.35) | | | |
| 7-[3-(3,5-dichlorophenyl)-3-(trifluoromethyl)-2,4-dihydropyrrol-5- | A | 1.308 | 583.1 |
| yl]-N-[[5-(difluoromethyl)pyrimidin-2-yl]methyl]indane-4- | | | |
| carboxamide (XIV.36) | | | |
| 7-[3-(3,5-dichloro-4-fluoro-phenyl)-3-(trifluoromethyl)-2,4- | A | 1.269 | 577.2 |
| dihydropyrrol-5-yl]-N-(1,1-dioxothiolan-3-yl)indane-4- | | | |
| carboxamide (XIV.37) | | | |
| 7-[3-(3,5-dichloro-4-fluoro-phenyl)-3-(trifluoromethyl)-2,4- | A | 1.324 | 543.1 |
| dihydropyrrol-5-yl]-N-(2-oxotetrahydrofuran-3-yl)indane- | | | |
| 4-carboxamide (XIV.38) | | | |
| 7-[3-(3,5-dichloro-4-fluoro-phenyl)-3-(trifluoromethyl)-2,4- | A | 1.510 | 546.0 |
| dihydropyrrol-5-yl]-N-tetrahydrothiophen-3-yl-indane-4- | | | |
| carboxamide (XIV.39) | | | |
| N-allyl-7-[5-(3,5-dichloro-4-fluoro-phenyl)-5-(trifluoromethyl)- | A | 1.440 | 500.8 |
| 4H-isoxazol-3-yl]indane-4-carboxamide (XIV.40) | | | |
| 7-[5-(3,5-dichlorophenyl)-5-(trifluoromethyl)-4H-isoxazol-3- | A | 1.317 | 535.9 |
| yl]-N′-pyrimidin-2-yl-indane-4-carbohydrazide (XIV.41) | | | |
| 7-[5-(3,5-dichlorophenyl)-5-(trifluoromethyl)-4H-isoxazol-3- | A | 1.396 | 556.0 |
| yl]-N-[(4S)-2-ethyl-3-oxo-isoxazolidin-4-yl]indane-4- | | | |
| carboxamide (XIV.42) | | | |
| 7-[5-(3,5-dichlorophenyl)-5-(trifluoromethyl)-4H-isoxazol-3- | A | 1.446 | 529.0 |
| yl]-N-(thietan-3-ylmethyl)indane-4-carboxamide (XIV.43) | | | |
| N-[(2,2-dichlorocyclopropyl)methyl]-7-[5-(3,5-dichloro- | A | 1.526 | 584.9 |
| 4-fluoro-phenyl)-5-(trifluoromethyl)-4H-isoxazol-3-yl]indane- | | | |
| 4-carboxamide (XIV.44) | | | |
| 7-[5-(3,5-dichloro-4-fluoro-phenyl)-5-(trifluoromethyl)-4H- | A | 1.373 | 557.0 |
| isoxazol-3-yl]-N-[(2-methyltetrazol-5-yl)methyl]indane-4- | | | |
| carboxamide (XIV.45) | | | |
| 7-[5-(3,5-dichloro-4-fluoro-phenyl)-5-(trifluoromethyl)-4H- | A | 1.375 | 542.9 |
| isoxazol-3-yl]-N-(1,2,4-oxadiazol-3-ylmethyl)indane-4- | | | |
| carboxamide (XIV.46) | | | |
| 7-[5-(3,5-dichloro-4-fluoro-phenyl)-5-(trifluoromethyl)-4H- | A | 1.399 | 547.1 |
| isoxazol-3-yl]-N-(1,3-dioxolan-2-ylmethyl)indane-4-carboxamide | | | |
| (XIV.47) | | | |
| 7-[5-(3,5-dichloro-4-fluoro-phenyl)-5-(trifluoromethyl)-4H- | A | 1.349 | 549.0 |
| isoxazol-3-yl]-N-(diethyl-λ⁴-sulfanylidene)indane-4-carboxamide | | | |
| (XIV.48) | | | |
| 7-[2-(3,5-dichloro-4-fluoro-phenyl)-2-(trifluoromethyl)-3H- | A | 1.464 | 590.4 |
| thiophen-4-yl]-N-[(4R)-2-ethyl-3-oxo-isoxazolidin-4-yl]indane- | | | |
| 4-carboxamide (XIV.49) | | | |
| 7-[2-(3,5-dichlorophenyl)-2-(trifluoromethyl)-3H-thiophen-4- | A | 1.461 | 561.9 |
| yl]-N-(1,1-dioxothietan-3-yl)indane-4-carboxamide (XIV.50) | | | |
| 7-[2-(3,5-dichloro-4-fluoro-phenyl)-2-(trifluoromethyl)-3H- | B | 1.428 | 579.6 |
| thiophen-4-yl]-N-(1,1-dioxothietan-3-yl)indane-4-carboxamide | | | |
| (XIV.51) | | | |

TABLE 6
Characterization of dihydrofurane compounds by HPLC-MS

| | | | Mass over |
|---|---|---|---|
| | HPLC | Retention | charge |
| Compound | set-up | time | (m/z) |
| 7-[2-(3,5-dichlorophenyl)-2-(trifluoromethyl)-3H-furan-4- | A | 1.388 | 555.1 |
| yl]-N-[(4R)-2-ethyl-3-oxo-isoxazolidin-4-yl]indane-4- | | | |
| carboxamide (XIV.52) | | | |
| 7-[2-(3,5-dichlorophenyl)-2-(trifluoromethyl)-3H-furan-4- | A | 1.370 | 581.0 |
| yl]-N-[2-oxo-2-(2,2,2-trifluoroethylamino)ethyl]indane-4- | | | |
| carboxamide (XIV.53) | | | |
| 7-[2-(3,5-dichlorophenyl)-2-(trifluoromethyl)-3H-furan-4- | A | 1.451 | 516.0 |
| yl]-N-(2-methylsulfanylethyl)indane-4-carboxamide (XIV.54) | | | |
| 7-[2-(3,5-dichlorophenyl)-2-(trifluoromethyl)-3H-furan-4- | A | 1.378 | 534.0 |
| yl]-N-(pyrimidin-2-ylmethyl)indane-4-carboxamide (XIV.55) | | | |

TABLE 7
HPLC set-up and equipment

| Code used in Tables | | |
|---|---|---|
| 3, 4, and 5 | HPLC set-up | Equipment |
| A | Mobile phase A: water + 0.1% TFA; mobile | MSD4/5: Shimadzu Nexera UHPLC and |
| | phase B: MeCN; gradient: 5% B to 100% B in | Shimadzu LCMS 20-20, ESI; |
| | 1.50 min, 100% B for 0.25 min; flow 0.8 mL/min | Column: Phenomenex Kinetex 1.7 μm XB- |
| | to 1 mL/min in 1.51 min; temperature 60° C.; ESI | C18 100A, 50 × 2.1 mm |
| | positive; range (m/z) 100-700 | |
| B | Mobile phase A: water + 0.1% TFA; mobile | |
| | phase B: MeCN; gradient: 5% B to 100% B in | |
| | 1.50 min, 100% B for 0.25 min; flow 0.8 mL/min | |
| | to 1 mL/min in 1.51 min; temperature 60° C.; ESI | |
| | positive; range (m/z) 50-700 | |
| C | Mobile phase A: water + 0.1% TFA; mobile | |
| | phase B: MeCN; gradient: 5% B to 100% B in | |
| | 1.50 min, 100% B for 0.25 min; flow 0.8 mL/min | |
| | to 1 mL/min in 1.51 min; temperature 60° C.; ESI | |
| | positive; range (m/z) 100-1400 | |

1. Compounds of formula I 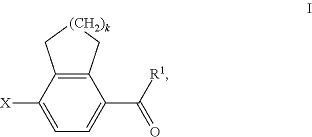
wherein the variables have the following meaning:
X Cl, Br, or I;
R¹ H, OR¹¹, or NR¹²R¹³;
R¹¹ a) H;
b) C₁-C₆-alkyl, C₃-C₈-cycloalkyl, C₂-C₆-alkenyl, C₃-C₈-cycloalkenyl, C₂-C₆-alkynyl;
which groups are unsubstituted, or substituted by halogen, CN, NO₂, phenyl, S(O)ₘR^{A}, OR^{B}, NR^{B}R^{C}, S(O)ₘNR^{B}R^{C}, Si(R^{B})₂R^{C}, C(═O)R^{B}, C(═O)NR^{B}R^{C}, C(═O)OR^{B}, C(═S)R^{B}, C(═S)NR^{B}R^{C}, C(═S)OR^{B}, C(═S)SR^{B}, C(═NR^{B})R^{C}, C(═NR^{B})NR^{C}R^{D};
c) phenyl, which is unsubstituted, or substituted by R^{A}; or
d) a 3-, 4-, 5-, 6-, or 7-membered saturated, partially unsaturated or fully unsaturated heterocycle, which heterocycle comprises one, or more, same, or different heteroatoms O, N(O)ₙ, or S(O)ₘ;
wherein
R^{A} a) C₁-C₆-alkyl, C₃-C₈-cycloalkyl-C₁-C₄-alkyl, C₁-C₄-alkyl-C₃-C₈-cycloalkyl, C₃-C₈-cycloalkyl, C₂-C₆-alkenyl, C₂-C₆-alkynyl, phenyl;
which groups are unsubstituted, or substituted by halogen, CN, OH, NO₂, phenyl, or C₁-C₆-alkyl-phenyl;
b) a 3-, 4-, 5-, 6-, or 7-membered saturated, partially unsaturated or fully unsaturated heterocycle, which heterocycle comprises one, or more, same, or different heteroatoms O, N(O)ₙ, and S(O)ₘ, wherein none, one, or more ring members are replaced by C(═O), or C(═S), and which heterocycle is unsubstituted, or substituted by halogen, CN, N₃, NO₂, SCN, SF₅, C₁-C₆-alkyl, C₁-C₆-alkoxy, C₁-C₆-haloalkyl, C₁-C₆-alkoxy-C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, or C₂-C₆-haloalkynyl;
R^{B}, R^{C}, R^{D} are independently from one another, as defined for R^{A}, or H; or
two substituents R^{B}, R^{C}, or R^{D}, together with the atom, or the atoms to which they are bound, form a 3, 4, 5, 6, or 7-membered saturated, partially unsaturated, or fully unsaturated carbocycle, or heterocycle, which cycles are unsubstituted, or substituted by R^{A}, and wherein the heterocycle comprises one, or more, same, or different heteroatoms O, N(O)ₙ, or S(O)ₘ, and wherein none, one, or more ring members are replaced by C(═O), or C(═S);
R¹² H, C₁-C₆-alkyl, C₂-C₆-alkenyl, C₂-C₆-alkynyl, C₃-C₈-cycloalkyl, phenyl; which groups are unsubstituted, or substituted by R^{E};
R¹³ a) H, C₁-C₆-alkyl, C₁-C₆-alkoxy, C₂-C₆-alkenyl, C₂-C₆-alkynyl, C₃-C₈-cycloalkyl, C₃-C₈-cycloalkyl-C₁-C₆-alkyl, phenyl; which groups are unsubstituted, or substituted by R^{E};
b) a group Z-A, wherein Z is a chemical bond, CH₂, CH₂CH₂ or C═O; and A is a 3-, 4-, 5-, 6- or 7-membered saturated, partially unsaturated or fully unsaturated heterocycle, which heterocycle is unsubstituted or substituted by R^{F} and comprises one, or more, same, or different heteroatoms O, N(O)ₙ, and S(O)ₘ, and wherein none, one, or more ring members are replaced by C(═O), or C(═S), C(═NR^{B}), or C(═NOR^{B});
c) a group S(O)ₘR^{A}, S(O)ₘN(R^{B})R^{C}, N(R^{B})R^{C}, N(R^{B})C(═O)OR^{C}, N(R^{B})C(═O)N(R^{C})R^{D}, N(R^{B})C(═S)OR^{C}, N(R^{B})C(═S)N(R^{C})R^{D}, C(═O)N(R^{B})R^{C}, C(═S)N(R^{B})R^{C}, C(═O)OR^{A}, C═NOR^{A}, C═NR^{A}R^{B}, C═NR^{B}R^{C}; or
wherein R¹² and R¹³, together with the N-atom to which they are bound, form a 3, 4, 5, 6, or 7-membered saturated, partially unsaturated, or fully unsaturated carbocycle, or heterocycle, which cycles are unsubstituted, or substituted by R^{F}, and wherein the heterocycle comprises one, or more, same, or different heteroatoms O, N(O)ₙ, and S(O)ₘ, and wherein none, one, or more ring members are replaced by C(═O), or C(═S), C(═NR^{B}), or C(═NOR^{B});
or wherein R¹² and R¹³, together with the N-atom to which they are bound, form a group ═S(R^{B})R^{C}, ═NR^{B}, ═NOR^{B}, or ═NN(R^{B})R^{C};
R^{E} a) halogen, CN, N₃, NO₂, SCN, SF₅, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, Si(R^{B})₂R^{C}, OR¹¹, OSO₂R^{A}, S(O)ₘR^{A}, S(O)ₘN(R^{B})R^{C}, N(R^{B})R^{C}, C(═O)N(R^{B})R^{C}, C(═O)N(R^{B})N(R^{C})R^{D}, C(═O)NOR^{B}, C(═S)N(R^{B})R^{C}, C(═O)OR^{A};
b) phenyl, which is unsubstituted, or substituted by R^{A}; or
c) two substituents R^{E}, together with the atom, or the atoms to which they are bound, form a 3, 4, 5, 6, or 7-membered saturated, partially unsaturated, or fully unsaturated carbocycle, or heterocycle, which cycles are unsubstituted, or substituted by R^{A}, and wherein the heterocycle comprises one, or more, same, or different heteroatoms O, N(O)ₙ, or S(O)ₘ, and wherein none, one, or more ring members are replaced by C(═O), or C(═S), C(═NR^{B}), or C(═NOR^{B});
R^{F} a) halogen, CN, N₃, NO₂, SCN, SF₅, C₁-C₆-alkyl, C₁-C₆-haloalkyl, C₁-C₆-alkoxy-C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, Si(R^{B})₂R^{C}, OR¹¹, OSO₂R^{A}, S(O)ₘR^{A}, S(O)ₘN(R^{B})R^{C}, N(R^{B})R^{C}, C(═O)N(R^{B})R^{C}, C(═O)N(R^{B})N(R^{C})R^{D}, C(═O)NOR^{B}, C(═S)N(R^{B})R^{C}, C(═O)OR^{A};
b) phenyl, which is unsubstituted, or substituted by R^{A}; or
c) two substituents R^{F}, together with the atom, or the atoms to which they are bound, form a 3, 4, 5, 6, or 7-membered saturated, partially unsaturated, or fully unsaturated carbocycle, or heterocycle, which cycles are unsubstituted, or substituted by R^{A}, and wherein the heterocycle comprises one, or more, same, or different heteroatoms O, N(O)ₙ, and S(O)ₘ, and wherein none, one, or more ring members are replaced by C(═O), or C(═S), C(═NR^{B}), or C(═NOR^{B});
k 1, or 2
m 0, 1, or 2;
n 0, or 1.

2. The compounds according to claim 1, wherein k is 1.

3. The compounds according to claim 1, wherein k is 2.

4. The compounds according to claim 1, wherein R¹ is OR¹¹, and R¹¹ is H, C₁-C₆-alkyl, phenyl, or benzyl.

5. The compounds according to claim 4, wherein R¹¹ is C₁-C₄-alkyl.

6. A process for the production of compounds I, as defined in claim 1, by reaction of compounds II with a reducing agent.

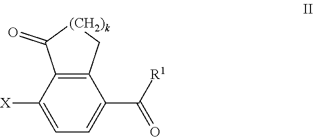

7. The process according to claim 6, wherein compounds II are produced by reaction of compounds V 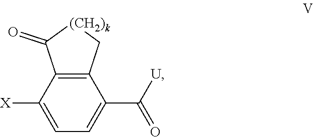
with H₂O, R¹¹OH, or NHR¹²R¹³; wherein X is Cl, Br, or I, and U is halogen; and
wherein compounds V are produced by reaction of compounds III 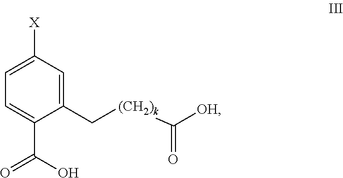
with a halogenating agent, followed by cyclization in the presence of a Lewis acid.

8. The process according to claim 7, wherein compounds III are produced by reaction of compounds IV selected from IVa, IVb, or IVc 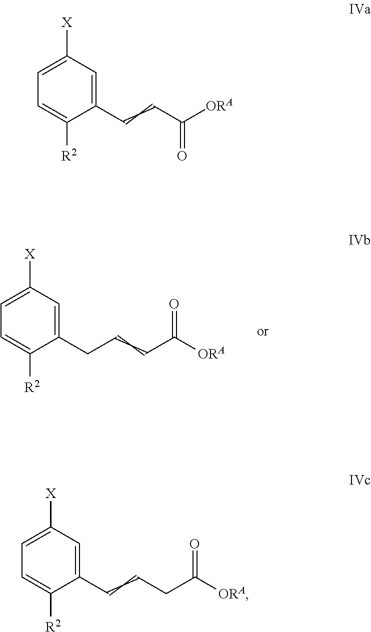
with hydrogen, followed by hydrolysis; wherein R² is CN, or C(═O)OR^{A}.

9. The process according to claim 8, wherein the hydrogen is produced in situ from
a) a metal selected from alkali metals, and alkaline earth metals, or
b) a metal with a redox potential below 0 at a pH below 7.0.

10. (canceled)

11. Compounds of formula VI 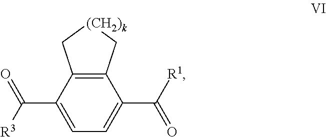
produced from compounds of formula 1 as defined in claim 1, wherein R³ is H, or CH₃, and k and R¹ have a meaning as defined in claim 1.

12. Insecticidal compounds of formula XIV-A or XV-A 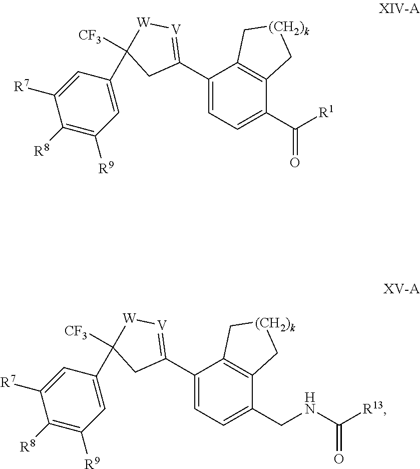
produced from compounds of formula 1 as defined in claim 1, wherein V is selected from CH, N, and NO, W is selected from O, S and CH₂, and R⁷, R⁸, and R⁹ are independently hydrogen, halogen, halomethyl, or halomethoxy, wherein at most two substituents R⁷, R⁸, and R⁹ are H, and R¹, R¹³, and k have a meaning as defined in claim 1.

13. (canceled)

14. (canceled)

15. (canceled)

16. The insecticidal compounds of claim 12, wherein the compounds of formula 1 are produced by reaction of compounds II with a reducing agent. [chemical structure]
